(19) United States
(12) Patent Application Publication
(10) Pub. No.: US 2026/0124324 A1
(43) Pub. Date: May 7, 2026

(54) PHOTOCLICK CHEMISTRY OF SILK FOR TEMPORARY UV TATTOOS

(71) Applicant: Trustees of Tufts College, Medford, MA (US)

(72) Inventors: Fiorenzo G. Omenetto, Lexington, MA (US); Nicholas Ostrovsky-Snider, Medford, MA (US)

(21) Appl. No.: 19/322,126

(22) Filed: Sep. 8, 2025

Related U.S. Application Data
(63) Continuation of application No. PCT/US24/19428, filed on Mar. 11, 2024.
(60) Provisional application No. 63/489,366, filed on Mar. 9, 2023.

(51) Int. Cl. A61K 49/00

(52) U.S. Cl. CPC A61K 49/006

(57) ABSTRACT

Silk photoclick tattoo inks include modified silk nanoparticles including modified silk fibroin functionalized with a plurality of first photoclick chemistry pair moieties, wherein the modified silk nanoparticles are biocompatible and bioresorbable; and photoclick chromophores including a second photoclick chemistry pair moiety, wherein the first photoclick chemistry pair moiety and the second photoclick chemistry pair moiety undergo a known photoreaction when illuminated with light having a predetermined wavelength for a predetermined exposure length and a predetermined exposure intensity, thereby covalently bonding at least a portion of the photoclick chromophores to at least a portion of the modified silk nanoparticles, wherein the silk photoclick tattoo ink is safe for human use as a tattoo ink.

### CLAIM TO PRIORITY

This application claims benefit of and is a continuation of International Patent Application No. PCT/US2024/019428 (Attorney Docket. No. 2095.0596), filed Mar. 11, 2024, and entitled “PHOTOCLICK CHEMISTRY OF SILK FOR TEMPORARY UV TATTOOS,” International Pub. No. WO 2024/187194, which is hereby incorporated by reference in its entirety for all purposes.

International Patent Application No. PCT/US2024/019428 relates to, incorporates by reference for all purposes, and claims priority to U.S. Application Ser. No. 63/489,366, filed Mar. 9, 2023.

### STATEMENT REGARDING FEDERALLY FUNDED RESEARCH

Not applicable.

### SEQUENCE LISTING

Not applicable.

### BACKGROUND

In the medical field there is a need for a transient, selectively visible tattoo, such as for use as a fiducial marker in teletherapy treatments. Teletherapy treatments are highly localized treatments of radiation that are often used for treatment of certain forms of cancer. Because of the localized nature of the treatment, the radiation beam needs to be precisely aligned with the tumor in order for the treatment to be effective and to minimize side effects. This is often done by performing detailed medical imaging such as CAT, PET or MRI scans of the patient, and placing one or more small tattoos on the skin in the ideal place to align the beam. This must be done as the imaging and treatment often cannot be performed simultaneously.

Currently, small permanent marks must be made in the patient's skin. These may be embarrassing to the patient, especially if they are on highly visible portions of the body. Additionally, tattoos can often be painful to apply using traditional methods, and require substantial training and practice to apply reliably.

Some fluorescent tattoo inks already exist, but these have at least two principal failings that this invention addresses. First, they are “permanent” tattoos in that they do not have programmable functional lifetimes. These particles degrade stochastically and slowly, so while the tattoo will lose its definition and luster, it will always be somewhat visible for the wearer's lifetime. Secondly, they do not have the ability to switch on their fluorescence post-injection, meaning they are not compatible with the ‘painless’ tattooing process described later in this disclosure. Thus, there is a need for a transient, selectively visible tattoo that can alleviate both problems.

When high-value items need to maintain authenticity after exchange through non-secure chains of custody it is common to include anticounterfeiting marks to prevent the items from being replaced by inferior duplicates. Historically anticounterfeiting marks have been created by processes that are very difficult or expensive to replicate (holographic images, fluorescent markings, watermarks, micro printing, etc.) however advances in technology have made these easier to reproduce and thus less secure. Physically unclonable functions (PUFs) are a relatively new form of anti-counterfeiting mark that utilize inherent degrees of randomness to make a mark that is both easy to initially produce and very difficult to replicate. This marking is then stored in a database and the marking on the item can be checked against this registry by individuals throughout the chain of custody to ensure that it has not been replaced by a counterfeit item.

A need exists for compositions and methods that overcome one or more of the aforementioned shortcomings.

### SUMMARY

In some aspects, the disclosure herein relates to a silk photoclick tattoo ink including: modified silk nanoparticles including modified silk fibroin functionalized with a plurality of first photoclick chemistry pair moieties, wherein the modified silk nanoparticles are biocompatible and bioresorbable; and photoclick chromophores including a second photoclick chemistry pair moiety, wherein the first photoclick chemistry pair moiety and the second photoclick chemistry pair moiety undergo a known photoreaction when illuminated with light having a predetermined wavelength for a predetermined exposure length and a predetermined exposure intensity, thereby covalently bonding at least a portion of the photoclick chromophores to at least a portion of the modified silk nanoparticles, wherein the silk photoclick tattoo ink is safe for human use as a tattoo ink.

In some aspects, the disclosure herein relates to a silk photoclick article ink including: modified silk nanoparticles including modified silk fibroin functionalized with a plurality of first photoclick chemistry pair moieties, wherein the modified silk nanoparticles are biocompatible and bioresorbable; and photoclick chromophores including a second photoclick chemistry pair moiety, wherein the first photoclick chemistry pair moiety and the second photoclick chemistry pair moiety undergo a known photoreaction when illuminated with light having a predetermined wavelength for a predetermined exposure length and a predetermined exposure intensity, thereby covalently bonding at least a portion of the photoclick chromophores to at least a portion of the modified silk nanoparticles.

In some aspects, the techniques described herein relate to a method of generating a silk photoclick tattoo in a subject's skin, the method including the following steps: a) administering a silk photoclick tattoo ink to an area of the subject's skin, the silk photoclick tattoo ink including modified silk nanoparticles and photoclick chromophores, the modified silk nanoparticles including modified silk fibroin functionalized with a plurality of first photoclick chemistry pair moieties, wherein the modified silk nanoparticles are biocompatible and bioresorbable, the photoclick chromophores including a second photoclick chemistry pair moiety; b) exposing the silk photoclick tattoo ink within the area to light having a predetermined wavelength for a predetermined exposure length and a predetermined exposure intensity, thereby initiating a known photoreaction and covalently bonding at least a portion of the photoclick chromophores to at least a portion of the modified silk nanoparticles, the administering and exposing of steps a) and b) thereby generating the silk photoclick tattoo in the area of the subject's skin.

In some aspects, the techniques described herein relate to a method of generating a silk photoclick image within a layer of an article, the method including the following steps: a) administering a silk photoclick article ink to an area of the layer of the article, the silk photoclick article ink including modified silk nanoparticles and photoclick chromophores, the modified silk nanoparticles including modified silk fibroin functionalized with a plurality of first photoclick chemistry pair moieties, wherein the modified silk nanoparticles are biocompatible and bioresorbable, the photoclick chromophores including a second photoclick chemistry pair moiety; b) exposing the silk photoclick article ink within the area to light having a predetermined wavelength for a predetermined exposure length and a predetermined exposure intensity, thereby initiating a known photoreaction and covalently bonding at least a portion of the photoclick chromophores to at least a portion of the modified silk nanoparticles, the administering and exposing of steps a) and b) thereby generating the silk photoclick image in the area of the layer of the article.

In some aspects, the techniques described herein relate to a method of generating a silk photoclick volumetric image within a volume of an article, the method including the following steps: a) administering a silk photoclick article ink to a volume, the silk photoclick article ink including modified silk nanoparticles and photoclick chromophores, the modified silk nanoparticles including modified silk fibroin functionalized with a plurality of first photoclick chemistry pair moieties, wherein the modified silk nanoparticles are biocompatible and bioresorbable, the photoclick chromophores including a second photoclick chemistry pair moiety; b) exposing the silk photoclick article ink within the volume to light having a predetermined wavelength for a predetermined exposure length and a predetermined exposure intensity, thereby initiating a known photoreaction and covalently bonding at least a portion of the photoclick chromophores to at least a portion of the modified silk nanoparticles, the administering and exposing of steps a) and b) thereby generating the silk photoclick volumetric image within the volume of the article.

These and other systems, methods, objects, features, and advantages of the present disclosure will be apparent to those skilled in the art from the following detailed description of the preferred embodiment and the drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE FIGURES

The disclosure and the following detailed description of certain embodiments thereof may be understood by reference to the following figures:

FIG. 1 depicts reaction schemes for making photoclick silk. A) The current library of explored 2,5-diaryltetrazole derivatives. B) A reaction schematic showing the photoclick reaction between DAT and dipolarophiles. C) A reaction scheme showing the methacrylation of silk fibroin with glycidyl methacrylate.

FIG. 2 depicts fluorescent tattoo formulations. A) A schematic illustration of the formulation of a ‘traditional tattoo ink’ from photoclicked silk. The Sil-MA nanoparticles are mixed with DAT, reacted under 302 nm illumination to for the fluorescent product then purified to get rid of unreacted DAT. This can then be tattooed in the traditional manner to produce a tattoo that is only visible under illumination by 365 nm light. B) A schematic illustration of the ‘inject-then-write’ tattoo method. A microneedle array is loaded with SilMA-NPs and DAT then injected into the patient. The injected area is patterned via patterned illumination creating fluorescent regions only where exposed, allowing those areas to fluoresce under 365 nm illumination.

FIG. 3 depicts photoclick tattoos on simulated skin. Top) A representation of a Bombyx mori silk moth with fluorescent patterns made by photoclick silk. Bottom) Two tattoos of the word “silk” made with photoclick silk ink, the left side was left untreated while the right side was exposed to 302 nm light to reveal the hidden message. Both demonstrate visibility only under 365 nm illumination.

FIG. 4 depicts a silk photoclick microsphere PUF. A) A fluorescence microscopy image of a microsphere laden film illuminated by 365 nm light and imaged in visible wavelengths. B) The green channel of the image was isolated and a 128×128 pixel section was isolated and C) converted to black and white by intensity thresholding. This black and white image was then converted into D) a 16 kbit binary marker that is unique to this PUF.

FIG. 5 depicts NMR analysis of DATs. The proton nuclear magnetic resonance spectra of DAT-5 in DMSO-d6 is shown with aromatic peaks showing all showing doublets. Acidic protons are seen far downfield.

FIG. 6A and FIG. 6B depict FT-IR spectrum of DAT-8. A) The complete mid-range infrared spectrum of DAT-8 showing clear amine peaks and B) a closer look at the 1700-900 cm⁻¹ region showing triple ring (1624 cm⁻¹, 1610 cm⁻¹, 1592 cm⁻¹) stretches from the two aryl rings and the tetrazole ring, the triple nitrogen stretch from the tetrazole core at 1308 cm⁻¹, and the region that holds the tetrazole-breathing mode signals that are not distinguishable from other signals in the fingerprint region.

FIGS. 7A, 7B, 7C, and 7D depict UV-Vis absorption spectra of DAT derivatives. Above are structures of A) DAT derivatives 1, and 6-10 and B) DAT derivatives 1-5. Below the structures are C) UV-Vis absorbance spectra of DAT derivatives 1, and 6-10, and D) DAT 1-5 with legends indicating what functional group is attached to the DAT.

FIG. 8 depicts numbering of pyrazoline atoms and labeling of pyrazoline derivatives. To the left is a general structure of the pyrazolines that result from DAT/PEG-DA photoclick reactions, with the atoms of the pyrazoline numbered. To the right are the labels of the various pyrazoline derivatives (Pyrs) that arise from reactions of DAT-1-10 with PEG-DA. Enumeration of DATs and Pyrs is congruent, so reaction of DAT-1 with PEG-DA produces Pyr-1.

FIG. 9 depicts PEG-DA-bound pyrazoline fluorescing in various solvents. Upper photos show fluorophores resulting from the reactions of DAT 1-10 with PEG-DA then dissolved in ethyl acetate (EtAc), isopropyl alcohol (IPA) and water. Below image shows functional group associated with each DAT molecule.

FIG. 10 depicts electron density on the 3-phenyl position alters fluorescence emission wavelengths. A) Pyrazoline derivatives that vary at the 3-phenyl position show B) a clear bathochromic shift when increasingly electron withdrawing substituents are added that C) shift the peak emission wavelengths from 430 to 600 nm. D) The positioning of the substituents at the meta position drastically alters the emission from electron withdrawing derivatives, but minimally shifts when an electron donating substituent is moved to the meta position. E) Shifting the functional group to the meta position shifts nitro derivatives from 600 to 450 nm, while amines only shift from 455 to 450.

FIG. 11 depicts effects of electron density altering substituents on the 1-phenyl ring. A) Pyrs 1-5 have varying substituents from strongly electron donating hydroxyls to strongly withdrawing sulfates. B) The fluorescent emissions show a general trend of bathochromic emission shifting with increasing electron donation, C) shifting from 470 with carbonyl substituents to 555 with a hydroxyl substituent.

FIG. 12 depicts a schematic of fluorescence titration measurement apparatus. Schematic shows a beaker on a stir plate, containing a magnetic stirrer, an immersed digital pH probe and an optical fiber. During titration the solution was illuminated with a 365 nm LED and the emission spectrum and pH were measured simultaneously after addition of acid or base.

FIG. 13 depicts fluorescence spectra of Pyr-1 in water and IPA pH titrations. A) shows the shift in emission spectra of Pyr-1 in water or B) isopropyl alcohol upon the addition of 1 M pTSA.

FIG. 14 depicts fluorescence titration plots. A) A plot of emission intensity at 500 nm versus pH for Pyr 1, 2, 3, 5, and 10, each displaying at least 1 pH dependent shift in emission with Pyr 5 display 2. B) A plot of emission intensity at 500 nm versus pH for Pyr 4, 6, 7, 8, and 9, all of which display insensitivity to pH. C) A linear fit of Pyr-1's linear region to back calculate the pKa of the derivative. D) A table of the calculated substituents and pKas of the various pyrazoline derivatives.

FIG. 15 depicts that changes in pH predictably change the emission of pyrazoline derivatives. A) Pyrazoline derivatives functionalized on the 3-phenyl position fluorescing in solution including ionized configurations that alter the electron density of the resulting pyrazoline. All derivatives pictured are in IPA except Pyr-6 (orange, —NO₂), which is shown in ethyl acetate as the derivative is quenched in IPA. B) Pyr-1 in an acidic environment has an electron withdrawing carboxylic acid, that becomes a mildly electron donating carboxylate in basic conditions, following the general trend. C) Electron donating amines at high pHs become withdrawing ammonium when going from basic to acidic conditions and consequently causing a bathochromic shift.

FIG. 16 depicts pH dependance of pyrazolines substituted on the 1-phenyl position. Photographs of pyrazolines in IPA labeled with their functional group at the para position on the 1-phenyl ring.

FIG. 17 depicts NMR analysis of silk fibroin before and after reaction with glycidyl methacrylate. A) 1H-NMR spectrum of unmodified silk fibroin and B) silk methacrylate. Green highlighted regions of the spectra correspond to methacrylate peaks that appear after reaction with glycidyl methacrylate. Protons corresponding to the signals highlighted in green are shown on the right. Orange highlighted area shows the shifts in aromatic peaks, likely corresponding to chemical modifications to tyrosine.

FIG. 18 depicts a comparison of SilMA- and PEG-DA-bound pyrazoline fluorescence. A) Photographs of pyrazolines derived from DAT-1-10 reacted to PEG-DA in IPA or SilMA in water. B) Emission spectra of SilMA-bound Pyr-1 in water most closely resemble PEG-DA-bound pyrazolines in IPA, compared to water or ethyl acetate. C) The emission spectra of Pyr-1 bound to SilMA shows greater similarity to Pyr-1 bound to PEG-DA in IPA rather than water.

FIG. 19 depicts the relationship of the molecular weight of silk fibroin with boiling time during degumming. A) Combined chromatographs of silk boiled between 10 and 180 minutes. B) The calculated molecular weights of silks boiled from 10 to 180 minutes, fitted with a logarithmic trendline, and C) plot of dispersity of samples error bars show standard deviation, n=3.

FIG. 20 depicts the photoclick reaction of DAT-Phenol after the Gomberg-Bachmann arylation. A) Reaction scheme of photoclick reaction of DAT-Phenol with PEG-DA. B) Filter paper blotted with DAT-Phenol, PEG-DA and both at the bottom, then exposed to 302 nm light and visualized under 365 nm light.

FIG. 21 depicts photopatterning of SilMA films with DATs. A) A plot of emission intensity over time of a SilMA film impregnated with DAT-1 and exposed to 302 nm for periods of time between 10 and 300 seconds through a quartz photomask and B) images of those films under 365 nm illumination. C) Images of SilMA and SF/PEG-DA films impregnated with DAT1-10 and exposed to 302 nm light for 30 seconds through a steel shadow mask.

FIG. 22 depicts fabrication of fluorescent films with multiple independent emission colors. A) A schematic of the process of creating multilayer, multi-emitter films. First a glass slide is coated with either SilMA or SF/PEG-DA and water vapor annealed. Next the film is impregnated with DAT by spin coating and the fluorescent pattern (here a QR code) can be applied by photo-exposure. The excess DAT is then washed away to prevent spurious exposures. If the initial layer was SilMA, a second layer can be applied in an identical process to the first. If SF/PEG-DA was used for the first layer, a PDMS isolation layer must be applied to prevent quenching. After the isolation layer is applied, the next layer may be applied in an identical manner to the first with a different pattern (a butterfly) with a different DAT precursor. B) Shows two films with a SilMA upper layer and but the left has a SF/PEG-DA lower layer, while the right has a SilMA underlay, resulting in the lower layer (QR code) being quenched on the left but visible on the right. C) Shows films with SF/PEG-DA lower layers with the addition of an isolation layer, enable upper layers to be applied without quenching.

FIG. 23 depicts multispectral imaging and emission isolation of patterned films. A) A multi-emitter film (Upper layer is SilMA patterned with DAT-5, lower layer is SilMA patterned with DAT 3) imaged with a multispectral camera showing (clockwise from the upper left) the true color image of the film, the spectrally isolated upper layer, the spectrally isolated lower layer, and a reconstructed image of the film from spectral components. B) A plot of the isolated spectral components with the upper layer in green and lower in blue.

FIG. 24 depicts emission spectra and environmental response of multilayer fluorescent films. A) Multispectral images of a multi-emitter film (SilMA-Pyr3 lower layer, SilMA-Pyr5 upper layer) both dry and in borate buffer (pH 10.0), with the spectral components of each layer isolated and displayed below. B) A plot of the emission of each spectral component showing quenching of the upper layer and a bathochromic shift of the lower layer by the borate buffer.

FIG. 25 depicts SEM measurement of SilMA NPs fabricated by acetone precipitation. A) Scanning electron micrograph of SilMA NPs fabricated by acetone precipitation. Scale bar is 1 μm. B) Histogram of SilMA NP sizes derived from SEM image analysis.

FIG. 26 depicts measurement of SilMA particle sizes. A) Electron micrograph of SilMA microparticles, scale bar is 10 μm. B) Histogram displays particle sizes by frequency in the SEM image derived by manual measurement. C) Optical micrograph of SilMA microparticles under bright field reflection. Scale bar is 25 μm. D) Histogram displays particle sizes by frequency in the image as counted by automatic particle analysis using Fiji software.

FIG. 27 depicts a comparison of diffusion of microparticle and aqueous inks. Shown are photographs agarose gel phantoms tattooed with the word “SILK” by an oscillating needle tattoo gun at various times after the tattoo was applied. Images on the left show microparticle bound dye is entirely resistant to diffusion while images on the right show aqueous SilMA-bound-pyrazoline dye is still able to diffuse in the medium.

FIG. 28 depicts DAT-fluorescent tattoos. Images of porcine skin that has been tattooed with DAT-loaded-SilMA-microparticles (light rectangle) then patterned by UV exposure through a shadow mask. A) Text is invisible under white light illumination but B) is revealed by 365 nm illumination. C) A tattoo of the word “silk” tattooed by hand vs D) a region nonspecifically tattooed with unreacted DAT ink then photopatterned.

FIG. 29 depicts SF microneedle arrays loaded with DAT-MPs. A) Photos of microneedle arrays loaded with DAT-MPs in visible light and B) under 365 nm illumination on an ungloved hand, C) under 365 nm illumination on gloved hand from a glancing and D) head-on perspective Scanning electron micrographs showing a silk microneedle array loaded with SilMA microparticles. The images show E) whole microneedles, F) a damaged microneedle revealing the microparticle cargo, and G) an enlarged image of that same microneedle. Scale bars are 100, 20 and 10 μm respectively.

FIG. 30 depicts delivery of microparticles by microneedle array. Z-resolved fluorescent microscopy of an agarose gel phantom with a SilMA microparticle loaded microneedle embedded in it. A) images show various rotations of a 3D reconstruction of the micrograph from the top, B) a three-quarters perspective and C) from the left side. D) Z-stack was merged to show a full focus reconstruction of the z-stack. E) Shows dot pattern resulting from microneedle tattoos and superimposition of multiple layers of the tattoos to effectively increase the resolution of the tattoo.

FIG. 31 depicts brightfield microscopy images of cells incubated with DAT-1. Brightfield microscopy of human dermal fibroblasts before and after 24 hour incubation with: unaugmented media, media augmented with Triton X-100, 1% DMSO, and DAT-1 at 4, 2, 1 mM concentrations. Contracted cells in 4 mM DAT-1 sample indicate moderate amount of cell death, with few dead cells in 2 mM and totally normal morphology in 1 mM and all lower concentration.

FIG. 32 depicts metabolic toxicity of DAT-1 as determined by 2D MTT assay. Cell metabolic rates measured by 2D MTT assay, rates normalized to media-only control, n=6. All groups were compared to negative controls by ANOVA with Tukey Post Test. Significance level is indicated by *, with all non-indicated comparisons being statistically insignificant.

FIG. 33 depicts microscopy of 3D skin-simulating scaffolds. Collagen coated silk scaffold that were seeded with hDFs then tattooed with DAT-MPs visualists in (left) bright-field transmission mode and (right) in fluorescence (ex/em: 358/461).

FIG. 34 depicts results from MTT assay on 3D scaffolds. 3D scaffolds were seeded with hDFs then tattooed with 7 injections of 1 μL of DPS, 1% w/v SilMA MPs in DPBS, Triton X-100, DAT-MPs that were pre-irradiated with 302 nm UV, and DAT-MPs that were never treated with 302 nm MPs. All groups were compared to the DPBS negative control via ANOVA with Tukey post-test, number of stars indicate significance level, all unindicated comparisons showed no significant difference from the negative control. Error bars show standard deviation, n=3.

FIG. 35 depicts fluorescent microscopy of tattooed 3D skin simulating scaffolds. Z-resolved fluorescence images of cells 7 days after tattooing with various inks. DAPI channels also show DAT and silk structure due to overlapping excitation/emission from DAT signal and silk autofluorescence. Phalloidin channel in DPBS and DAT treated samples show elongate cell bodies in the pores of the scaffold structure. DAPI and Phalloidin channels show only background due to non-specific adsorption to silk, indicating no living cells are present. All scale bars are 100 μm.

### DETAILED DESCRIPTION

Before the present disclosure is described in further detail, it is to be understood that the disclosure is not limited to the particular embodiments described. It is also understood that the terminology used herein is for the purpose of describing particular embodiments only, and is not intended to be limiting. The scope of the present disclosure will be limited only by the claims. As used herein, the singular forms “a”, “an”, and “the” include plural embodiments unless the context clearly dictates otherwise.

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” are used as equivalents and may be understood to permit standard variation as would be understood by those of ordinary skill in the art; and (v) where ranges are provided, endpoints are included.

Approximately: as used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Composition: as used herein, may be used to refer to a discrete physical entity that comprises one or more specified components. In general, unless otherwise specified, a composition may be of any form—e.g., gas, gel, liquid, solid, etc. In some embodiments, “composition” may refer to a combination of two or more entities for use in a single embodiment or as part of the same article. It is not required in all embodiments that the combination of entities result in physical admixture, that is, combination as separate co-entities of each of the components of the composition is possible; however many practitioners in the field may find it advantageous to prepare a composition that is an admixture of two or more of the ingredients in a pharmaceutically acceptable carrier, diluent, or excipient, making it possible to administer the component ingredients of the combination at the same time.

Improve, increase, or reduce: as used herein or grammatical equivalents thereof, indicate values that are relative to a baseline measurement, such as a measurement in a similar composition made according to previously known methods.

Substantially: as used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, “silk fibroin” refers to silk fibroin protein whether produced by silkworm, spider, or other insect, or otherwise generated (Lucas et al., Adv. Protein Chem., 13: 107-242 (1958)). Any type of silk fibroin can be used in different embodiments described herein. Silk fibroin produced by silkworms, such as Bombyx mori, is the most common and represents an earth-friendly, renewable resource. For instance, silk fibroin used in a silk film may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants, and variants thereof, that can be used. See, e.g., WO 97/08315 and U.S. Pat. No. 5,245,012, each of which is incorporated herein by reference in their entireties.

Disclosed herein are photoclick tattoos. Photoclick tattoos can alleviate the embarrassment of tattoos on highly visible body parts because the tattoo is only visible under the select condition of UV-A illumination. This means that outside of the operating room or other setting with the relevant conditions, they would be almost undetectable, while inside the operating room, or other relevant location, they could be detected with a simple hand-held UV-A flashlight. This wavelength of UV is safe for short exposures, and it is often used by entertainment venues as “black light”, which excites fluorescent chemicals present in clothing and causes them to glow.

The invention could also remove the pain of tattooing. These dyes can be injected in their already-fluorescent state using a pain-free method such as microneedle arrays. Microneedle arrays can be fabricated from silk and can be designed to penetrate the dermis deep enough to deposit the ink but shallow enough not to trigger pain signals from nerve endings in the skin. Alternatively, this photoclick reaction allows for the non-fluorescent precursors to be injected into the skin and patterned after injection. Once in the skin, the injected dye can be selectively turned fluorescent only in the desired region by activating the photoclick reaction through a photomask. The unreacted dye will be rapidly resorbed by the body, leaving only the activated regions. This removes the need for the physician or other practitioner to accurately pattern the mark with a traditional tattoo gun. It also alleviates the need to pre-pattern the microneedle arrays which enables far easier scale-up.

Disclosed herein is silk photoclick tattoo ink. Examples of silk photoclick tattoo ink disclosed herein include modified silk nanoparticles comprising modified silk fibroin functionalized with a plurality of first photoclick chemistry pair moieties. The modified silk nanoparticles are biocompatible and bioresorbable. Examples of silk photoclick tattoo ink disclosed herein also include photoclick chromophores including a second photoclick chemistry pair moiety, wherein the first photoclick chemistry pair moiety and the second photoclick chemistry pair moiety undergo a known photoreaction when illuminated with light having a predetermined wavelength for a predetermined exposure length and a predetermined exposure intensity, thereby covalently bonding at least a portion of the photoclick chromophores to at least a portion of the modified silk nanoparticles.

The silk photoclick tattoo ink is safe for human use as a tattoo ink. Silk photoclick ink can undergo rigorous testing for microorganisms, pathogens, and other microbial contaminants in the same way that conventional tattoo ink is tested, including testing for one of the most common bacteria found in contaminated tattoo ink, nontuberculous mycobacteria (NTM), as well as Pseudomonas aeruginosa, Bacillus cereus, and other microorganisms. In some embodiments, silk photoclick ink has less than 1 CFU/100 mL, less than 0.5 CFU/100 mL, or less than 0.25 CFU/100 mL of any one microbial contaminant. Silk photoclick ink can also undergo processing to ensure the absence of microorganisms, pathogens, and other potential contaminants in the same way that conventional tattoo ink is processed, including exposure to radiation (e.g., gamma radiation). In embodiments, silk photoclick ink is exposed to gamma radiation until the silk photoclick ink has less than 1 CFU/100 mL, less than 0.5 CFU/100 mL, or less than 0.25 CFU/100 mL of any one microbial contaminant. Silk photoclick ink may have low or no levels of allergens or certain heavy metals typical of conventional tattoo ink, such as mercury, cadmium, lead, manganese, or chromium. Silk photoclick ink may have low or no levels of certain impurities typical of conventional tattoo ink, such as polycyclic aromatic hydrocarbons, aromatic amines, formaldehyde, parabens, benzothiazolinone, and isothiazolinones.

Disclosed herein is an example silk photoclick article ink including modified silk nanoparticles. The modified silk nanoparticles include modified silk fibroin functionalized with a plurality of first photoclick chemistry pair moieties, wherein the modified silk nanoparticles are biocompatible and bioresorbable, and photoclick chromophores including a second photoclick chemistry pair moiety. The first photoclick chemistry pair moiety and the second photoclick chemistry pair moiety undergo a known photoreaction when illuminated with light having a predetermined wavelength for a predetermined exposure length and a predetermined exposure intensity, thereby covalently bonding at least a portion of the photoclick chromophores to at least a portion of the modified silk nanoparticles. Also disclosed herein are dissolvable microneedles or microneedle arrays including the silk photoclick tattoo ink or the silk photoclick article ink disclosed herein.

Disclosed herein is a method of generating a silk photoclick tattoo in a subject's skin including administering a silk photoclick tattoo ink to an area of the subject's skin. In embodiments, administering may be via a dissolvable microneedle. The silk photoclick tattoo ink includes modified silk nanoparticles, which are biocompatible and bioresorbable, and photoclick chromophores. The modified silk nanoparticles include modified silk fibroin functionalized with a plurality of first photoclick chemistry pair moieties. The photoclick chromophores include a second photoclick chemistry pair moiety. The method also includes exposing the silk photoclick tattoo ink within the area to light having a predetermined wavelength for a predetermined exposure length and a predetermined exposure intensity, thereby initiating a known photoreaction and covalently bonding at least a portion of the photoclick chromophores to at least a portion of the modified silk nanoparticles. The steps of administering and exposing thereby generate the silk photoclick tattoo in the area of the subject's skin.

In some examples, the method further includes administering the silk photoclick tattoo ink to a delimited area of the subject's skin. In some examples, the silk photoclick tattoo ink that is administered within the delimited area of the area of the subject's skin has spectroscopic or optical properties that differs from the silk photoclick tattoo ink that is not within the delimited area. In other examples, the spectroscopic or optical properties of the silk photoclick tattoo ink are the same throughout the area of the subject's skin, including within the delimited area. Differing spectroscopic or optical properties of silk photoclick tattoo ink may result in differences in color, luminescence, fluorescence, phosphorescence, or the like. The method further includes preventing the silk photoclick tattoo ink within the delimited area from undergoing the known photoreaction. The method may further include optionally waiting a bioresorption length of time, wherein the administering, preventing, and optionally waiting steps of the method result in generating an absence of the silk photoclick tattoo in the delimited area. In many cases, an absence of a tattoo or an image itself defines the image. The boundary of an image is defined as much by the presence of ink in one pixel/voxel as the absence of the ink in the next pixel/voxel. The bioresorption length of time is between 1 month and 1 year, including but not limited to, between 4 to 6 months. In examples, the step of preventing the silk photoclick tattoo ink within the delimited area from undergoing the known photoreaction includes applying a photomask to the delimited area, wearing a protective sheath covering the delimited area, selectively illuminating (e.g. limiting exposure by use of digital light projection or a scanned focused illumination source such as a laser), or a combination thereof.

In some examples, the method further includes administering a selective degradation agent to at least a portion of the area, thereby dissolving at least a portion of the silk photoclick tattoo ink within the at least a portion of the area, thereby reducing the visibility of the silk photoclick tattoo within the at least a portion of the area. The selective degradation agent may be an enzymatic agent, such as protease XIV, trypsin, bromelain, papain, or a combination thereof.

In some examples, the method further includes administering two different inks to two different areas, wherein the two different inks have different optical properties. This is also possible with three different inks and three different areas, four different inks with four different areas, and so on.

In some examples, the method includes selectively administering inks with different optical properties to different areas, thereby generating images with varying optical properties across the different areas.

Disclosed herein is a method of generating a silk photoclick image within a layer of an article including administering a silk photoclick article ink to an area of the layer of the article and exposing the silk photoclick article ink within the area to light thereby generating the silk photoclick image in the area of the layer of the article. The silk photoclick article ink includes modified silk nanoparticles and photoclick chromophores. The modified silk nanoparticles include modified silk fibroin functionalized with a plurality of first photoclick chemistry pair moieties. The modified silk nanoparticles are biocompatible and bioresorbable, and the photoclick chromophores include a second photoclick chemistry pair moiety. In examples, exposing the silk photoclick article ink within the area includes exposing to light having a predetermined wavelength for a predetermined exposure length and a predetermined exposure intensity, thereby initiating a known photoreaction and covalently bonding at least a portion of the photoclick chromophores to at least a portion of the modified silk nanoparticles. The steps of administering and exposing thereby generate the silk photoclick image in the area of the layer of the article. In examples, the method may further include administering the silk photoclick article ink to a delimited area of the layer of the article, and preventing the silk photoclick article ink within the delimited area from undergoing the known photoreaction, which generates an absence of the silk photoclick image in the delimited area. The step of preventing may include applying a photomask to the delimited area, wearing a protective sheath covering the delimited area, selectively illuminating (e.g. limiting exposure by use of digital light projection or a scanned focused illumination source such as a laser), or a combination thereof. In some examples, a selective degradation agent may be administered to at least a portion of the area, thereby dissolving at least a portion of the silk photoclick article ink within the at least a portion of the area, thereby reducing the visibility of the silk photoclick image within the at least a portion of the area. The selective degradation agent may be an enzymatic agent, such as protease XIV, trypsin, bromelain, papain, or a combination thereof. In some examples, the method includes administering two different inks to two different volumes, wherein the two different inks have different optical properties. In some examples, the method includes selectively administering inks with different optical properties to different areas, thereby generating volumetric images with varying optical properties across the different areas.

Disclosed herein is a method of generating a silk photoclick volumetric image within a volume of an article including administering a silk photoclick article ink to a volume, and exposing the silk photoclick article ink within the volume to light having a predetermined wavelength for a predetermined exposure length and a predetermined exposure intensity, thereby initiating a known photoreaction and covalently bonding at least a portion of the photoclick chromophores to at least a portion of the modified silk nanoparticles. The silk photoclick article ink includes modified silk nanoparticles and photoclick chromophores, and the modified silk nanoparticles include modified silk fibroin functionalized with a plurality of first photoclick chemistry pair moieties, wherein the modified silk nanoparticles are biocompatible and bioresorbable, and the photoclick chromophores include a second photoclick chemistry pair moiety. The steps of administering and exposing thereby generate the silk photoclick volumetric image within the volume of the article. In examples, the method further includes administering the silk photoclick article ink to a delimited volume of the article and preventing the silk photoclick article ink within the delimited volume from undergoing the known photoreaction,
the administering and preventing thereby generating an absence of the silk photoclick volumetric image in the delimited volume. In examples, the step of preventing includes applying a photomask to the delimited area, wearing a protective sheath covering the delimited area, selectively illuminating (e.g. limiting exposure by use of digital light projection or a scanned focused illumination source such as a laser), or a combination thereof. In examples, the method further includes administering a selective degradation agent to at least a portion of the volume, thereby dissolving at least a portion of the silk photoclick article ink within the at least a portion of the volume, thereby reducing the visibility of the silk photoclick volumetric image within the at least a portion of the volume. The selective degradation agent may be an enzymatic agent, such as protease XIV, trypsin, bromelain, papain, or a combination thereof. In some examples, the method includes administering two different inks to two different volumes, wherein the two different inks have different optical properties. In some examples, the method includes selectively administering inks with different optical properties to different volumes, thereby generating volumetric images with varying optical properties across the different volumes.

In any of the example silk photoclick tattoo inks, silk photoclick article inks, or methods herein, the modified silk nanoparticles can have an average particle diameter of between 0.1 and 50 micrometers, including but not limited to between 1 and 3 micrometers.

In any of the example silk photoclick tattoo inks, silk photoclick article inks, or methods herein, the modified silk nanoparticles can have an average volumetric density of the plurality of first photoclick chemistry pair moieties of 10-15%. In examples, 10-15% of the volume of the microparticles are composed of pyrazoline (the fluorescent compound) in a fully-reacted sample. The particles react volumetrically as they are homogenous and the dye precursor molecules penetrate into the silk particles to react.

In any of the example silk photoclick tattoo inks, silk photoclick article inks, or methods herein, the plurality of first photoclick chemistry pair moieties includes methacrylate moieties and the second photoclick chemistry pair moiety is a diaryl-tetrazole moiety.

In any of the example silk photoclick tattoo inks, silk photoclick article inks, or methods herein, the second photoclick chemistry pair moiety is a 2,5-diaryl-tetrazole moiety.

In any of the example silk photoclick tattoo inks, silk photoclick article inks, or methods herein, the covalent bonding activates a fluorescence within the silk photoclick tattoo ink.

In any of the example silk photoclick tattoo inks, silk photoclick article inks, or methods herein, the photoclick chromophores have the following formula:

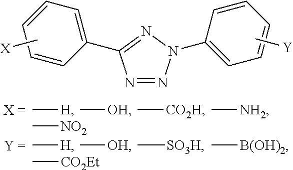

In examples, the X and Y substituents may include ethers, esters, cyano groups, -secondary -tertiary and -quaternary amines, halides, thiols, halogenated methyl groups, or aliphatic chains at either X or Y positions and with various regio-chemical arrangements (i.e. ortho vs meta vs para). In some examples, one or both phenyl rings are replaced with naphthyl rings, which themselves can be substituted with the above functional groups.

In any of the example silk photoclick tattoo inks, silk photoclick article inks, or methods herein, the photoclick chromophores are selected from the following group:

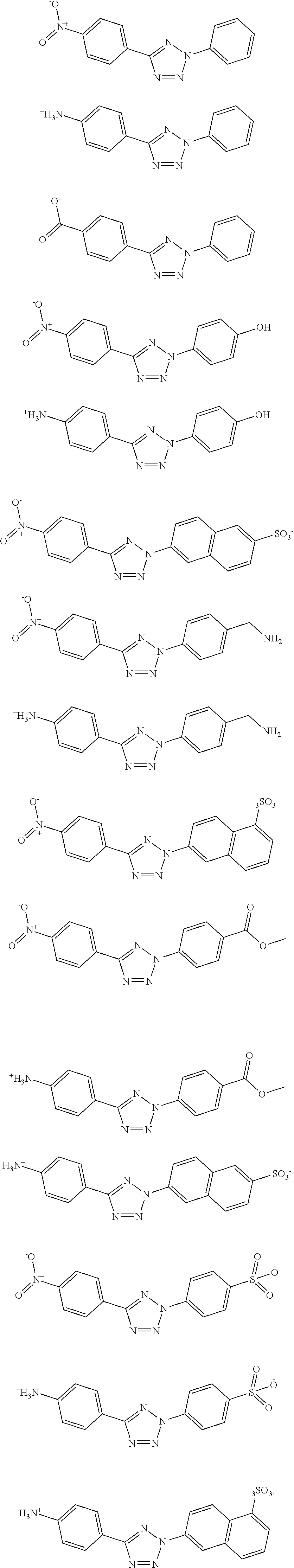

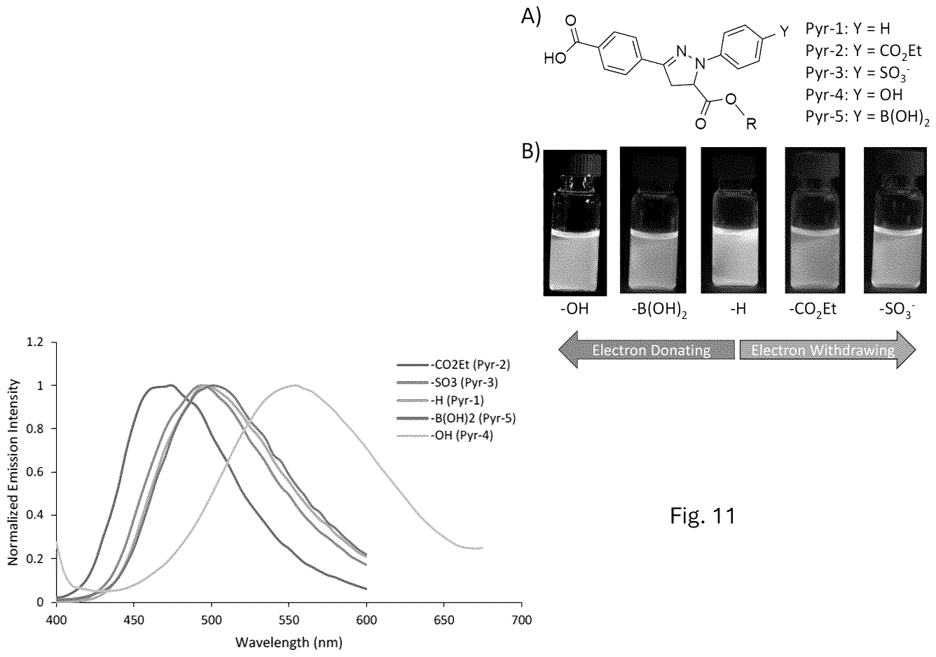

In examples of generating specific optical properties, the emission color is directly related to the electronegativity and regiochemistry of the X and Y substituents. (See Ostrovsky-Snider (2003), Chapter 2, pages 50-60, FIGS. 11-18). If ionizable substituents are used (e.g., a carboxylic acid) then the emission color can also be changed by altering the environmental pH.

In one specific aspect, an article prepared as described herein can be a physically unclonable function (PUF).

Also disclosed herein are silk-based physically unclonable functions by incorporating fluorescent silk microparticles into silk films. These microparticles are made fluorescent by a tetrazole-ene photoclick reaction that produces fluorescent pyrazoline adducts covalently attached to the silk microparticles with no toxic catalysts or biproducts. These allow printing of non-fluorescent spheres and selective fluorescent patterning to produce both visible patterning from the position of spheres and fluorescent patterning from the selective photoclicking of certain regions. Combined these produce both an added degree of security and a challenge-response pair in a biologically inert form for an edible PUF.

The security of these PUFs may be improved by the incorporation of multiple colors of emitters to create spectral distinction between various points in the image. These emitters may also be functionalized with ionizable functional groups that allow for pH dependent tuning of the emission wavelength and intensity of the microspheres.

A PUF may be as simple as the patterns of reflections from glitter encapsulated in transparent epoxy, which has a tremendous number of degrees of freedom ensuring every fabricated piece will be unique. This PUF is trivial to produce as any result will be acceptable and yet is quite difficult to replicate as exactly producing a specified pattern of glitter in the epoxy is exceptionally challenging. However, if the item at hand is sufficiently valuable it may still be profitable for counterfeiters to go to extraordinary lengths to replicate the PUF. With high precision printers and microfabrication technology both continuously advancing and increasing in availability, simple spatial patterning may prove insufficient to prevent counterfeiting in the near future. The next generation of PUFs will also need to incorporate active challenge-response mechanisms to increase the difficulty of reproduction.

Challenge-response pairs incorporate a physical mechanism that elicits a known response when a physical or chemical stimulus is applied. This can take the form of luminescence, chromatic shifts, physical shape changes, and more. These responses require highly specialized the PUF to have highly specific physical and chemical properties that create a new dimensional axis for degrees of freedom and thus both increase the possibility space and the difficulty in reproduction.

Biologically inert and even edible PUFs will also be needed soon to prevent counterfeiting of medicines. Many novel medications for utilize biologics, which are both highly effective and difficult to produce thus making these drugs command a high price. The potential profits make counterfeit drugs a distinct possibility, and the harm this would cause makes preventing counterfeiting a priority for drug manufacturers and healthcare providers alike. Thus, there is a need for PUFs that could be placed on the medicines directly and safely ingested by the patient, but that could also be documented by the healthcare provider to ensure the authenticity of the drug. These PUFs would need to be made of non-toxic and biologically inert materials to ensure they did not harm the patient or interact with the drug compound.

According to various embodiments, a variety of functionalizing agents may be used with the silk-containing embodiments described herein (e.g., silk membrane, silk composition, silk matrix, silk foam, silk microsphere, etc.). It should be understood that the examples herein may recite one or a few silk-containing embodiments but are applicable to any silk-containing embodiment, as applicable. In some embodiments, a functionalizing agent may be any compound or molecule that facilitates the attachment to and/or development (e.g., growth) of one or more endothelial cells on a silk membrane. In some embodiments, a functionalizing agent may be any compound or molecule that facilitates the attachment and/or development (e.g., growth) of one or more megakaryocytes and/or hematopoietic progenitor cells on a silk matrix and/or silk membrane. In some embodiments, a functionalizing agent may be or comprise an agent suitable for facilitating the production of one or more of white blood cells and red blood cells.

In some embodiments, a functionalizing agent may be or comprise a cell attachment mediator and/or an extracellular matrix protein, for example: collagen (e.g., collagen type I, collagen type III, collagen type IV or collagen type VI), elastin, fibronectin, vitronectin, laminin, fibrinogen, von Willebrand factor, proteoglycans, decorin, perlecan, nidogen, hyaluronan, and/or peptides containing known integrin binding domains e.g. “RGD” integrin binding sequence, or variations thereof, that are known to affect cellular attachment.

In some embodiments, a functionalizing agent may be any soluble molecule produced by endothelial cells. Non-limiting examples include fibroblast growth factor-1 (FGF1) and vascular endothelial growth factors (VEGF).

According to some embodiments, a plurality of functionalizing agents may be used. For example, in some embodiments wherein production of platelets is desired, provided compositions may comprise the use of laminin, fibronectin and/or fibrinogen, and type IV collagen in order to facilitate the attachment and growth of endothelial cells on a silk membrane (e.g., a porous silk membrane) and/or attachment of megakaryocytes to a silk matrix.

In some embodiments, a functionalizing agent may be embedded or otherwise associated with a silk membrane and/or silk matrix such that at least a portion of the functionalizing agent is surrounded by a silk membrane and/or silk matrix as contrasted to a functionalizing agent simply being positioned along the surface of a silk membrane and/or silk matrix. In some embodiments, a functionalizing agent is distributed along and/or incorporated in substantially the entire surface area of a silk membrane/silk wall. In some embodiments, a functionalizing agent is distributed and/or incorporated only at one or more discrete portions of a silk membrane/wall and/or silk matrix. In some embodiments, a functionalizing agent is distributed in and/or along at least one of the lumen-facing side of a silk wall and the matrix-facing side of a silk wall.

According to various embodiments, any application-appropriate amount of one or more functionalizing agents may be used. In some embodiments, the amount of an individual functionalizing agent may be between about 1 g/ml and 1,000 g/ml (e.g., between about 2 and 1,000, 5 and 1,000, 10 and 1,000, 10 and 500, 10 and 100 g/ml). In some embodiments, the amount of an individual functionalizing agent may be at least 1 g/ml (e.g., at least 5, 10, 15, 20 25, 50, 100, 200, 300 400, 500, 600, 700, 800, or 900 g/ml). In some embodiments, the amount of an individual functionalizing agent is at most 1,000 g/ml (e.g., 900, 800, 700, 600, 500, 400, 300 200, 100, 90, 80, 70, 60, 50, 40, 30, 20, 10, or 5 g/ml).

In some aspects, the composition comprises one or more sensing agents, such as a sensing dye. The sensing agents/sensing dyes are environmentally sensitive and produce a measurable response to one or more environmental factors. In some aspects, the environmentally-sensitive agent or dye may be present in the composition in an effective amount to alter the composition from a first chemical-physical state to a second chemical-physical state in response to an environmental parameter (e.g., a change in pH, light intensity or exposure, temperature, pressure or strain, voltage, physiological parameter of a subject, and/or concentration of chemical species in the surrounding environment) or an externally applied stimulus (e.g., optical interrogation, acoustic interrogation, and/or applied heat). In some cases, the sensing dye is present to provide one optical appearance under one given set of environmental conditions and a second, different optical appearance under a different given set of environmental conditions. Suitable concentrations for the sensing agents described herein can be the concentrations for the colorants and additives described elsewhere herein. A person having ordinary skill in the chemical sensing arts can determine a concentration that is appropriate for use in a sensing application of the inks described herein.

In some aspects, the first and second chemical-physical state may be a physical property of the composition, such as mechanical property, a chemical property, an acoustical property, an electrical property, a magnetic property, an optical property, a thermal property, a radiological property, or an organoleptic property. Exemplary sensing dyes or agents include, but are not limited to, a pH sensitive agent, a thermal sensitive agent, a pressure or strain sensitive agent, a light sensitive agent, or a potentiometric agent.

Exemplary pH sensitive dyes or agents include, but are not limited to, cresol red, methyl violet, crystal violet, ethyl violet, malachite green, methyl green, 2-(p-dimethylaminophenylazo) pyridine, paramethyl red, metanil yellow, 4-phenylazodiphenylamine, thymol blue, metacresol purple, orange IV, 4-o-Tolylazo-o-toluindine, quinaldine red, 2,4-dinitrophenol, erythrosine disodium salt, benzopurpurine 4B, N,N-dimethyl-p-(m-tolylazo) aniline, p-dimethylaminoazobenene, 4,4′-bis(2-amino-1-naphthylazo)-2,2′-stilbenedisulfonic acid, tetrabromophenolphthalein ethyl ester, bromophenol blue, Congo red, methyl orange, ethyl orange, 4-(4-dimethylamino-1-naphylazo)-3-methoxybenesulfonic acid, bromocresol green, resazurin, 4-phenylazo-1-napthylamine, ethyl red 2-([-dimethylaminophenyazo) pyridine, 4-(p-ethoxypehnylazo)-m-phenylene-diamine monohydrochloride, resorcin blue, alizarin red S, methyl red, propyl red, bromocresol purple, chlorophenol red, p-nitrophenol, alizarin 2-(2,4-dinitrophenylazo) 1-napthol-3,6-disulfonic acid, bromothymol blue, 6,8-dinitro-2,4-(1H) quinazolinedione, brilliant yellow, phenol red, neutral red, m-nitrophenol, cresol red, turmeric, metacresol purple, 4,4′-bis(3-amino-1-naphthylazo)-2,2′-stilbenedisulfonic acid, thymol blue, p-naphtholbenzein, phenolphthalein, o-cresolphthalein, ethyl bis(2,4-dimethylphenyl) ethanoate, thymolphthalein, nitrazine yellow, alizarin yellow R, alizarin, p-(2,4-dihydroxyphenylazo) benzenesulfonic acid, 5,5′-indigodisulfonic acid, 2,4,6-trinitrotoluene, 1,3,5-trinitrobenezne, and clayton yellow.

Exemplary light responsive dyes or agents include, but are not limited to, photochromic compounds or agents, such as triarylmethanes, stilbenes, azasilbenes, nitrones, fulgides, spiropyrans, napthopyrans, spiro-oxzines, quinones, derivatives and combinations thereof.

Exemplary potentiometric dyes include, but are not limited to, substituted amiononaphthylehenylpridinium (ANEP) dyes, such as di-4-ANEPPS, di-8-ANEPPS, and N-(4-Sulfobutyl)-4-(6-(4-(Dibutylamino)phenyl)hexatrienyl)Pyridinium (RH237).

Exemplary temperature sensitive dyes or agents include, but are not limited to, thermochromic compounds or agents, such as thermochromic liquid crystals, leuco dyes, fluoran dyes, octadecylphosphonic acid.

Exemplary pressure or strain sensitive dyes or agents include, but are not limited to, spiropyran compounds and agents.

Exemplary chemi-sensitive dyes or agents include, but are not limited to, antibodies such as immunoglobulin G (IgG) which may change color from blue to red in response to bacterial contamination.

In some aspects, the compositions comprise one or more additive, dopant, or biologically active agent suitable for a desired intended purpose. In some aspects, the additive or dopant may be present in the composition in an amount effective to impart an optical or organoleptic property to the composition. Exemplary additives or dopants that impart optical or organoleptic properties include, but are not limited to, dyes/pigments, flavorants, aroma compounds, granular or fibrous fillers.

Additionally or alternatively, the additive, dopant, or biologically active agent may be present in the composition in an amount effective to “functionalize” the composition to impart a desired mechanical property or added functionality to the composition. Exemplary additive, dopants, or biologically active agent that impart the desired mechanical property or added functionality include, but are not limited to: environmentally sensitive/sensing dyes; active biomolecules; conductive or metallic particles; micro and nanofibers (e.g., silk nanofibers for reinforcement, carbon nanofibers); nanotubes; inorganic particles (e.g., hydroxyapatite, tricalcium phosphate, bioglasses); drugs (e.g., antibiotics, small molecules or low molecular weight organic compounds); proteins and fragments or complexes thereof (e.g., enzymes, antigens, antibodies and antigen-binding fragments thereof); DNA/RNA (e.g., siRNA, miRNA, mRNA); cells and fractions thereof (viruses and viral particles; prokaryotic cells such as bacteria; eukaryotic cells such as mammalian cells and plant cells; fungi).

In some aspects, the additive or dopant comprises a flavoring agent or flavorant.

Exemplary flavorants include ester flavorants, amino acid flavorants, nucleic acid flavorants, organic acid flavorants, and inorganic acid flavorants, such as, but not limited to, diacetyl, acetylpropionyl, acetoin, isoamyl acetate, benzaldehyde, cinnamaldehyde, ethyl propionate, methyl anthranilate, limonene, ethyl decadienoate, allyl hexanoate, ethyl maltol, ethylvanillin, methyl salicylate, manzanate, glutamic acid salts, glycine salts, guanylic acids salts, inosinic acid salts, acetic acid, ascorbic acid, citric acid, fumaric acid, lactic acid, malic acid, phosphoric acid, tartaric acid, derivatives, and mixtures thereof.

In some aspects, the additive or dopant comprises an aroma compound. Exemplary aroma compounds include ester aroma compounds, terpene aroma compounds, cyclic terpenes, and aromatic aroma compounds, such as, but not limited to, geranyl acetate, methyl formate, methyl acetate, methyl propionate, methyl butyrate, ethyl acetate, ethyl butyrate, isoamyl acetate, pentyl butrate, pentyl pentanoate, octyl acetate, benzyl acetate, methyl anthranilate, myrecene, geraniol, nerol, citral, cironellal, cironellol, linalool, nerolidol, limonene, camphor, menthol, carone, terpineol, alpha-lonone, thujone, eucalyptol, benzaldehyde, eugenol, cinnamaldehyde, ethyl maltol, vanillin, anisole, anethole, estragole, thymol.

In some aspects, the additive or dopant comprises a colorant, such as a dye or pigment. In some aspects, the dye or pigment imparts a color or grayscale to the composition. The colorant can be different than the sensing agents and/or sensing dyes below. Any organic and/or inorganic pigments and dyes can be included in the inks. Exemplary pigments suitable for use in the present disclosure include International Color Index or C.I. Pigment Black Numbers 1, 7, 1 1 and 31, C.I. Pigment Blue Numbers 15, 15:1, 15:2, 15:3, 15:4, 15:6, 16, 27, 29, 61 and 62, C.I. Pigment Green Numbers 7, 17, 18 and 36, C.I. Pigment Orange Numbers 5, 13, 16, 34 and 36, C.I. Pigment Violet Numbers 3, 19, 23 and 27, C.I. Pigment Red Numbers 3, 17, 22, 23, 48:1, 48:2, 57:1, 81:1, 81:2, 81:3, 81:5, 101, 1 14, 122, 144, 146, 170, 176, 179, 181, 185, 188, 202, 206, 207, 210 and 249, C.I. Pigment Yellow Numbers 1, 2, 3, 12, 13, 14, 17, 42, 65, 73, 74, 75, 83, 30, 93, 109, 1 10, 128, 138, 139, 147, 142, 151, 154 and 180, D&C Red No. 7, D&C Red No. 6 and D&C Red No. 34, carbon black pigment (such as Regal 330, Cabot Corporation), quinacridone pigments (Quinacridone Magenta (228-0122), available from Sun Chemical Corporation, Fort Lee, N.J.), diarylide yellow pigment (such as AAOT Yellow (274-1788) available from Sun Chemical Corporation); and phthalocyanine blue pigment (such as Blue 15:3 (294-1298) available from Sun Chemical Corporation). The classes of dyes suitable for use in present invention can be selected from acid dyes, natural dyes, direct dyes (either cationic or anionic), basic dyes, and reactive dyes. The acid dyes, also regarded as anionic dyes, are soluble in water and mainly insoluble in organic solvents and are selected, from yellow acid dyes, orange acid dyes, red acid dyes, violet acid dyes, blue acid dyes, green acid dyes, and black acid dyes. European Patent 0745651, incorporated herein by reference, describes a number of acid dyes that are suitable for use in the present disclosure. Exemplary yellow acid dyes include Acid Yellow 1 International Color Index or C.I. 10316); Acid Yellow 7 (C.I. 56295); Acid Yellow 17 (C.I. 18965); Acid Yellow 23 (C.I. 19140); Acid Yellow 29 (C.I. 18900); Acid Yellow 36 (C.I. 13065); Acid Yellow 42 (C.I. 22910); Acid Yellow 73 (C.I. 45350); Acid Yellow 99 (C.I. 13908); Acid Yellow 194; and Food Yellow 3 (C.I. 15985). Exemplary orange acid dyes include Acid Orange 1 (C.I. 13090/1); Acid Orange 10 (C.I. 16230); Acid Orange 20 (C.I. 14603); Acid Orange 76 (C.I. 18870); Acid Orange 142; Food Orange 2 (C.I. 15980); and Orange B.

Exemplary red acid dyes include Acid Red 1. (C.I. 18050); Acid Red 4 (C.I. 14710); Acid Red 18 (C.I. 16255), Acid Red 26 (C.I. 16150); Acid Red 2.7 (C.I. as Acid Red 51 (C.I. 45430, available from BASF Corporation, Mt. Olive, N.J.) Acid Red 52 (C.I. 45100); Acid Red 73 (C.I. 27290); Acid Red 87 (C. I. 45380); Acid Red 94 (C.I. 45440) Acid Red 194; and Food Red 1 (C.I. 14700). Exemplary violet acid dyes include Acid Violet 7 (C.I. 18055); and Acid Violet 49 (C.I. 42640). Exemplary blue acid dyes include Acid Blue 1 (C.I. 42045); Acid Blue 9 (C.I. 42090); Acid Blue 22 (C.I. 42755); Acid Blue 74 (C.I. 73015); Acid Blue 93 (C.I. 42780); and Acid Blue 158A (C.I. 15050). Exemplary green acid dyes include Acid Green 1 (C.I. 10028); Acid Green 3 (C.I. 42085); Acid Green 5 (C.I. 42095); Acid Green 26 (C.I. 44025); and Food Green 3 (C.I. 42053). Exemplary black acid dyes include Acid Black 1 (C.I. 20470); Acid Black 194 (Basantol® X80, available from BASF Corporation, an azo/1:2 CR-complex.

Exemplary direct dyes for use in the present disclosure include Direct Blue 86 (C.I. 74180); Direct Blue 199; Direct Black 168; Direct Red 253; and Direct Yellow 107/132 (C.I. Not Assigned).

Exemplary natural dyes for use in the present disclosure include Alkanet (C.I. 75520, 75530); Annafto (C.I. 75120); Carotene (C.I. 75130); Chestnut; Cochineal (C.I. 75470); Cutch (C.I. 75250, 75260); Divi-Divi; Fustic (C.I. 75240); Hypernic (C.I. 75280); Logwood (C.I. 75200); Osage Orange (C.I. 75660); Paprika; Quercitron (C.I. 75720); Sanrou (C.I. 75100); Sandal Wood (C.I. 75510, 75540, 75550, 75560); Sumac; and Tumeric (C.I. 75300). Exemplary reactive dyes for use in the present disclosure include Reactive Yellow 37 (monoazo dye); Reactive Black 31 (disazo dye); Reactive Blue 77 (phthalo cyanine dye) and Reactive Red 180 and Reactive Red 108 dyes. Suitable also are the colorants described in The Printing Ink Manual (5th ed., Leach et al. eds. (2007), pages 289-299. Other organic and inorganic pigments and dyes and combinations thereof can be used to achieve the colors desired.

In addition to or in place of visible colorants, compositions provided herein can contain ETV fluorophores that are excited in the ETV range and emit light at a higher wavelength (typically 400 nm and above). Examples of ETV fluorophores include but are not limited to materials from the coumarin, benzoxazole, rhodamine, napthalimide, perylene, benzanthrones, benzoxanthones or benzothia-xanthones families. The addition of a UV fluorophore (such as an optical brightener for instance) can help maintain maximum visible light transmission. The amount of colorant, when present, generally is between 0.05% to 5% or between 0.1% and 1% based on the weight of the composition.

For non-white compositions, the amount of pigment/dye generally is present in an amount of from at or about 0.1 wt % to at or about 20 wt % based on the weight of the composition. In some applications, a non-white ink can include 15 wt % or less pigment/dye, or 10 wt % or less pigment/dye or 5 wt % pigment/dye, or 1 wt % pigment/dye based on the weight of the composition. In some applications, a non-white ink can include 1 wt % to 10 wt %, or 5 wt % to 15 wt %, or 10 wt % to 20 wt % pigment/dye based on the weight of the composition. In some applications, a non-white ink can contain an amount of dye/pigment that is 1 wt %, 2 wt %, 3 wt %, 4 wt %, 5%, 6 wt %, 7 wt %, 8 wt %, 9 wt %, 10 wt %, 11 wt %, 12 wt %, 13 wt %, 14 wt %, 15%, 16 wt %, 17 wt %, 18 wt %, 19 wt % or 20 wt % based on the weight of the composition.

For white compositions, the amount of white pigment generally is present in an amount of from at or about 1 wt % to at or about 60 wt % based on the weight of the composition. In some applications, greater than 60 wt % white pigment can be present. Preferred white pigments include titanium dioxide (anatase and rutile), zinc oxide, lithopone (calcined coprecipitate of barium sulfate and zinc sulfide), zinc sulfide, blanc fixe and alumina hydrate and combinations thereof, although any of these can be combined with calcium carbonate. In some applications, a white ink can include 60 wt % or less white pigment, or 55 wt % or less white pigment, or 50 wt % white pigment, or 45 wt % white pigment, or 40 wt % white pigment, or 35 wt % white pigment, or 30 wt % white pigment, or 25 wt % white pigment, or 20 wt % white pigment, or 15 wt % white pigment, or 10 wt % white pigment, based on the weight of the composition. In some applications, a white ink can include 5 wt % to 60 wt %, or 5 wt % to 55 wt %, or 10 wt % to 50 wt %, or 10 wt % to 25 wt %, or 25 wt % to 50 wt %, or 5 wt % to 15 wt %, or 40 wt % to 60 wt % white pigment based on the weight of the composition. In some applications, a non-white ink can an amount of dye/pigment that is 5%, 6 wt %, 7 wt %, 8 wt %, 9 wt %, 10 wt %, 11 wt %, 12 wt %, 13 wt %, 14 wt %, 15%, 16 wt %, 17 wt %, 18 wt %, 19 wt %, 20 wt %, 21 wt %, 22 wt %, 23 wt %, 24 wt %, 25%, 26 wt %, 27 wt %, 28 wt %, 29 wt %, 30 wt %, 31 wt %, 32 wt %, 33 wt %, 34 wt %, 35%, 36 wt %, 37 wt %, 38 wt %, 39 wt %, 40 wt %, 41 wt %, 42 wt %, 43 wt %, 44 wt %, 45%, 46 wt %, 47 wt %, 48 wt %, 49 wt %, 50 wt %, 51 wt %, 52 wt %, 53 wt %, 54 wt %, 55%, 56 wt %, 57 wt %, 58 wt %, 59 wt % or 60 wt % based on the weight of the composition.

In some aspects, the additive or dopant comprises a conductive additive. Exemplary conductive additives include, but are not limited to graphite, graphite powder, carbon nanotubes, and metallic particles or nanoparticles, such as gold nanoparticles. In some aspects, the conductive additive is biocompatible and non-toxic.

In some aspects, the additive is a biologically active agent. The term “biologically active agent” as used herein refers to any molecule which exerts at least one biological effect in vivo. For example, the biologically active agent can be a therapeutic agent to treat or prevent a disease state or condition in a subject. Biologically active agents include, without limitation, organic molecules, inorganic materials, proteins, peptides, nucleic acids (e.g., genes, gene fragments, gene regulatory sequences, and antisense molecules), nucleoproteins, polysaccharides, glycoproteins, and lipoproteins. Classes of biologically active compounds that can be incorporated into the composition provided herein include, without limitation, anticancer agents, antibiotics, analgesics, anti-inflammatory agents, immunosuppressants, enzyme inhibitors, antihistamines, anti-convulsants, hormones, muscle relaxants, antispasmodics, ophthalmic agents, prostaglandins, anti-depressants, anti-psychotic substances, trophic factors, osteoinductive proteins, growth factors, and vaccines.

The term “active agent” may also be used herein to refer to a biological sample (e.g., a sample of tissue or fluid, such as for instance blood) or a component thereof, and/or to a biologically active entity or compound, and/or to a structurally or functionally labile entity.

Exemplary active agents include, but are not limited to, therapeutic agents, diagnostic agents (e.g., contrast agents), and any combinations thereof. In some embodiments, the active agent present in a silk matrix (e.g., a silk microsphere), composition, or the like can include a labile active agent, e.g., an agent that can undergo chemical, physical, or biological change, degradation and/or deactivation after exposure to a specified condition, e.g., high temperatures, high humidity, light exposure, and any combinations thereof. In some embodiments, the active agent present in the silk matrix (e.g., a silk microsphere), composition, or the like can include a temperature-sensitive active agent, e.g., an active agent that will lose at least about 30% or more, of its original activity or bioactivity, upon exposure to a temperature of at least about 10° C. or above, including at least about 15° C. or above, at least about room temperature or above, or at least about body temperature (e.g., about 37° C.) or above.

The active agent can be generally present in the silk matrix (e.g., a silk microsphere), composition, or the like in an amount of about 0.01% (w/w) to about 70% (w/w), or about 0.1% (w/w) to about 50% (w/w), or about 1% (w/w) to about 30% (w/w). The active agent can be present on a surface of the silk matrix (e.g., a silk microsphere), composition, or the like and/or encapsulated and dispersed in the silk matrix (e.g., a silk microsphere), composition, or the like homogeneously or heterogeneously or in a gradient. In some embodiments, the active agent can be added into the silk solution, which is then subjected to the methods described herein for preparing a silk matrix (e.g., a silk microsphere), composition, or the like. In some embodiments, the active agent can be coated on a surface of the silk matrix (e.g., a silk microsphere), composition, or the like. In some embodiments, the active agent can be loaded in a silk matrix (e.g., a silk microsphere), composition, or the like by incubating the silk microsphere in a solution of the active agent for a period of time, during which an amount of the active agent can diffuse into the silk matrix (e.g., a silk microsphere), composition, or the like, and thus distribute within the silk matrix (e.g., a silk microsphere), composition, or the like.

In some aspects, the additive is a therapeutic agent. As used herein, the term “therapeutic agent” means a molecule, group of molecules, complex or substance administered to an organism for diagnostic, therapeutic, preventative medical, or veterinary purposes. As used herein, the term “therapeutic agent” includes a “drug” or a “vaccine.” This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a therapeutic effect, for example deoxyribonucleic acid (DNA), ribonucleic acid (RNA), nucleic acid analogues (e.g., locked nucleic acid (LNA), peptide nucleic acid (PNA), xeno nucleic acid (XNA)), or mixtures or combinations thereof, including, for example, DNA nanoplexes, siRNA, microRNA, shRNA, aptamers, ribozymes, decoy nucleic acids, antisense nucleic acids, RNA activators, and the like. Generally, any therapeutic agent can be included in the composition provided herein.

The term “therapeutic agent” also includes an agent that is capable of providing a local or systemic biological, physiological, or therapeutic effect in the biological system to which it is applied. For example, the therapeutic agent can act to control infection or inflammation, enhance cell growth and tissue regeneration, control tumor growth, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. Additionally, a silk-based drug delivery composition can contain one therapeutic agent or combinations of two or more therapeutic agents.

A therapeutic agent can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; an extract made from biological materials such as bacteria, plants, fungi, or animal cells; animal tissues; naturally occurring or synthetic compositions; and any combinations thereof. In some aspects, the therapeutic agent is a small molecule.

The term “bioactivity,” as used herein in reference to an active agent, generally refers to the ability of an active agent to interact with a biological target and/or to produce an effect on a biological target. For example, bioactivity can include, without limitation, elicitation of a stimulatory, inhibitory, regulatory, toxic or lethal response in a biological target. The biological target can be a molecule or a cell. For example, a bioactivity can refer to the ability of an active agent to modulate the effect/activity of an enzyme, block a receptor, stimulate a receptor, modulate the expression level of one or more genes, modulate cell proliferation, modulate cell division, modulate cell morphology, or any combination thereof. In some instances, a bioactivity can refer to the ability of a compound to produce a toxic effect in a cell. Exemplary cellular responses include, but are not limited to, lysis, apoptosis, growth inhibition, and growth promotion; production, secretion, and surface expression of a protein or other molecule of interest by the cell; membrane surface molecule activation including receptor activation; transmembrane ion transports; transcriptional regulations; changes in viability of the cell; changes in cell morphology; changes in presence or expression of an intracellular component of the cell; changes in gene expression or transcripts; changes in the activity of an enzyme produced within the cell; and changes in the presence or expression of a ligand and/or receptor (e.g., protein expression and/or binding activity). Methods for assaying different cellular responses are well known to one of skill in the art, e.g., western blot for determining changes in presence or expression of an endogenous protein of the cell, or microscopy for monitoring the cell morphology in response to the active agent, or FISH and/or qPCR for the detection and quantification of changes in nucleic acids. Bioactivity can be determined in some embodiments, for example, by assaying a cellular response.

In reference to an antibody, the term “bioactivity” includes, but is not limited to, epitope or antigen binding affinity, the in vivo and/or in vitro stability of the antibody, the immunogenic properties of the antibody, e.g., when administered to a human subject, and/or the ability to neutralize or antagonize the bioactivity of a target molecule in vivo or in vitro. The aforementioned properties or characteristics can be observed or measured using art-recognized techniques including, but not limited to, scintillation proximity assays, ELISA, ORIGEN immunoassay (IGEN), fluorescence quenching, fluorescence ELISA, competitive ELISA, SPR analysis including, but not limited to, SPR analysis using a BIAcore biosenser, in vitro and in vivo neutralization assays (see, for example, International Publication No. WO 2006/062685), receptor binding, and immunohistochemistry with tissue sections from different sources including human, primate, or any other source as needed. In reference to an immunogen, the “bioactivity” includes immunogenicity, the definition of which is discussed in detail later. In reference to a virus, the “bioactivity” includes infectivity, the definition of which is discussed in detail later. In reference to a contrast agent, e.g., a dye, the “bioactivity” refers to the ability of a contrast agent when administered to a subject to enhance the contrast of structures or fluids within the subject's body. The bioactivity of a contrast agent also includes, but is not limited to, its ability to interact with a biological environment and/or influence the response of another molecule under certain conditions.

As used herein, the term “small molecule” can refer to compounds that are “natural product-like,” however, the term “small molecule” is not limited to “natural product-like” compounds. Rather, a small molecule is typically characterized in that it contains several carbon carbon bonds, and has a molecular weight of less than 5000 Daltons (5 kDa), preferably less than 3 kDa, still more preferably less than 2 kDa, and most preferably less than 1 kDa. In some cases it is preferred that a small molecule have a molecular weight equal to or less than 700 Daltons.

Exemplary therapeutic agents include, but are not limited to, those found in Harrison's Principles of Internal Medicine, 13th Edition, Eds. T. R. Harrison et al. McGraw-Hill N.Y., NY; Physicians' Desk Reference, 50th Edition, 1997, Oradell New Jersey, Medical Economics Co.; Pharmacological Basis of Therapeutics, 8th Edition, Goodman and Gilman, 1990; United States Pharmacopeia, The National Formulary, ETSP XII NF XVII, 1990, the complete contents of all of which are incorporated herein by reference.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an anti arrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

Anti-cancer agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelinA receptor antagonists, retinoic acid receptor agonists, immuno-modulators, hormonal and antihormonal agents, photodynamic agents, and tyrosine kinase inhibitors.

Antibiotics include aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillinV, methicillin, natcillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocyline), and trimethoprim. Also included are metronidazole, fluoroquinolones, and ritampin.

Enzyme inhibitors are substances which inhibit an enzymatic reaction. Examples of enzyme inhibitors include edrophonium chloride, N-methylphysostigmine, neostigmine bromide, physostigmine sulfate, tacrine, tacrine, 1-hydroxy maleate, iodotubercidin, p-bromotetramiisole, lO-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N°-monomethyl-Larginine acetate, carbidopa, 3-hydroxybenzylhydrazine, hydralazine, cl orgyline, deprenyl, hydroxylamine, iproniazid phosphate, 6-MeO-tetrahydro-9H-pyrido-indole, nialamide, pargyline, quinacrine, semi carb azide, tranylcypromine, N,N-diethylaminoethyl-2,2-diphenylvalerate hydrochloride, 3-isobutyl-1-methylxanthne, papaverine, indomethacind, 2-cyclooctyl-2-hydroxy ethylamine hydrochloride, 2,3-dichloro-a-methylbenzylamine (DCMB), 8,9-dichloro-2,3,4, 5-tetrahydro-1H-2-benzazepine hydrochloride, p-amino glutethimide, p-aminoglutethimide tartrate, 3-iodotyrosine, alpha-methyltyrosine, acetazolamide, dichlorphenamide, 6-hydroxy-2-benzothiazolesulfonamide, and allopurinol.

Antihistamines include pyrilamine, chlorpheniramine, and tetrahydrazoline, among others.

Anti-inflammatory agents include corticosteroids, nonsteroidal anti-inflammatory drugs (e.g., aspirin, phenylbutazone, indomethacin, sulindac, tolmetin, ibuprofen, piroxicam, and fenamates), acetaminophen, phenacetin, gold salts, chloroquine, D-Penicillamine, methotrexate colchicine, allopurinol, probenecid, and sulfinpyrazone.

Muscle relaxants include mephenesin, methocarbomal, cyclobenzaprine hydrochloride, trihexylphenidyl hydrochloride, levodopa/carbidopa, and biperiden.

Anti-spasmodics include atropine, scopolamine, oxyphenonium, and papaverine.

Analgesics include aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocain, tetracaine and dibucaine.

Ophthalmic agents include sodium fluorescein, rose bengal, methacholine, adrenaline, cocaine, atropine, alpha-chymotrypsin, hyaluronidase, betaxalol, pilocarpine, timolol, timolol salts, and combinations thereof.

Prostaglandins are art recognized and are a class of naturally occurring chemically related long-chain hydroxy fatty acids that have a variety of biological effects.

Anti-depressants are substances capable of preventing or relieving depression.

Examples of anti-depressants include imipramine, amitriptyline, nortriptyline, protriptyline, desipramine, amoxapine, doxepin, maprotiline, tranylcypromine, phenelzine, and isocarboxazide.

Trophic factors are factors whose continued presence improves the viability or longevity of a cell trophic factors include, without limitation, platelet-derived growth factor (PDGP), neutrophil-activating protein, monocyte chemoattractant protein, macrophage-inflammatory protein, platelet factor, platelet basic protein, and melanoma growth stimulating activity; epidermal growth factor, transforming growth factor (alpha), fibroblast growth factor, platelet-derived endothelial cell growth factor, insulin-like growth factor, glial derived growth neurotrophic factor, ciliary neurotrophic factor, nerve growth factor, bone growth/cartilage-inducing factor (alpha and beta), bone morphogenetic proteins, interleukins (e.g., interleukin inhibitors or interleukin receptors, including interleukin 1 through interleukin 10), interferons (e.g., interferon alpha, beta and gamma), hematopoietic factors, including erythropoietin, granulocyte colony stimulating factor, macrophage colony stimulating factor and granulocyte-macrophage colony stimulating factor; tumor necrosis factors, and transforming growth factors (beta), including beta-1, beta-2, beta-3, inhibin, and activin.

Hormones include estrogens (e.g., estradiol, estrone, estriol, diethylstibestrol, quinestrol, chlorotrianisene, ethinyl estradiol, mestranol), anti-estrogens (e.g., clomiphene, tamoxifen), progestins (e.g., medroxyprogesterone, norethindrone, hydroxyprogesterone, norgestrel), antiprogestin (mifepristone), androgens (e.g, testosterone cypionate, fluoxymesterone, danazol, testolactone), anti-androgens (e.g., cyproterone acetate, flutamide), thyroid hormones (e.g., triiodothyronne, thyroxine, propylthiouracil, methimazole, and iodixode), and pituitary hormones (e.g., corticotropin, sumutotropin, oxytocin, and vasopressin). Hormones are commonly employed in hormone replacement therapy and/or for purposes of birth control. Steroid hormones, such as prednisone, are also used as immunosuppressants and anti-inflammatories. In some aspects, the additive is an agent that stimulates tissue formation, and/or healing and regrowth of natural tissues, and any combinations thereof. Agents that increase formation of new tissues and/or stimulates healing or regrowth of native tissue at the site of injection can include, but are not limited to, fibroblast growth factor (FGF), transforming growth factor-beta (TGF-beta, platelet-derived growth factor (PDGF), epidermal growth factors (EGFs), connective tissue activated peptides (CTAPs), osteogenic factors including bone morphogenic proteins, heparin, angiotensin II (A-II) and fragments thereof, insulin-like growth factors, tumor necrosis factors, interleukins, colony stimulating factors, erythropoietin, nerve growth factors, interferons, biologically active analogs, fragments, and derivatives of such growth factors, and any combinations thereof.

In some aspects, the silk composition can further comprise at least one additional material for soft tissue augmentation, e.g., dermal filler materials, including, but not limited to, poly(methyl methacrylate) microspheres, hydroxylapatite, poly(L-lactic acid), collagen, elastin, and glycosaminoglycans, hyaluronic acid, commercial dermal filler products such as BOTOX® (from Allergan), DYSPORT®, COSMODERM®, EVOLENCE®, RADIESSE®, RESTYLANE®, JUVEDERM® (from Allergan), SCULPTRA®, PERLANE®, and CAPTIQEIE®, and any combinations thereof.

In some aspects, the additive is a wound healing agent. As used herein, a “wound healing agent” is a compound or composition that actively promotes wound healing process.

Exemplary wound healing agents include, but are not limited to dexpanthenol; growth factors; enzymes, hormones; povidon-iodide; fatty acids; anti-inflammatory agents; antibiotics; antimicrobials; antiseptics; cytokines; thrombin; angalgesics; opioids; aminoxyls; furoxans; nitrosothiols; nitrates and anthocyanins; nucleosides, such as adenosine; and nucleotides, such as adenosine diphosphate (ADP) and adenosine triphosphate (ATP); neutotransmitter/neuromodulators, such as acetylcholine and 5-hydroxytryptamine (serotonin/5-HT); histamine and catecholamines, such as adrenalin and noradrenalin; lipid molecules, such as 5 sphingosine-1-phosphate and lysophosphatidic acid; amino acids, such as arginine and lysine; peptides such as the bradykinins, substance P and calcium gene-related peptide (CGRP); nitric oxide; and any combinations thereof.

In certain aspects, the active agents provided herein are immunogens. In one aspect, the immunogen is a vaccine. Most vaccines are sensitive to environmental conditions under which they are stored and/or transported. For example, freezing may increase reactogenicity (e.g., capability of causing an immunological reaction) and/or loss of potency for some vaccines (e.g., HepB, and DTaP/IPV/FQB), or cause hairline cracks in the container, leading to contamination. Further, some vaccines (e.g., BCG, Varicella, and MMR) are sensitive to heat. Many vaccines (e.g., BCG, MMR, Varicella, Meningococcal C Conjugate, and most DTaP-containing vaccines) are light sensitive. See, e.g., Galazka et ak, Thermostability of vaccines, in Global Programme for Vaccines & Immunization (World Health Organization, Geneva, 1998); Peetermans et al, Stability of freeze-dried rubella virus vaccine (Cendehill strain) at various temperatures, 1 J. Biological Standardization 179 (1973). Thus, the compositions and methods provided herein also provide for stabilization of vaccines regardless of the cold chain and/or other environmental conditions.

In some aspects, the additive is a cell, e.g., a biological cell. Cells useful for incorporation into the composition can come from any source, e.g., mammalian, insect, plant, etc. In some aspects, the cell can be a human, rat or mouse cell. In general, cells to be used with the compositions provided herein can be any types of cells. In general, the cells should be viable when encapsulated within compositions. In some aspects, cells that can be used with the composition include, but are not limited to, mammalian cells (e.g. human cells, primate cells, mammalian cells, rodent cells, etc.), avian cells, fish cells, insect cells, plant cells, fungal cells, bacterial cells, and hybrid cells. In some aspects, exemplary cells that can be used with the compositions include platelets, activated platelets, stem cells, totipotent cells, pluripotent cells, and/or embryonic stem cells. In some aspects, exemplary cells that can be encapsulated within compositions include, but are not limited to, primary cells and/or cell lines from any tissue. For example, cardiomyocytes, myocytes, hepatocytes, keratinocytes, melanocytes, neurons, astrocytes, embryonic stem cells, adult stem cells, hematopoietic stem cells, hematopoietic cells (e.g. monocytes, neutrophils, macrophages, etc.), ameloblasts, fibroblasts, chondrocytes, osteoblasts, osteoclasts, neurons, sperm cells, egg cells, liver cells, epithelial cells from lung, epithelial cells from gut, epithelial cells from intestine, liver, epithelial cells from skin, etc, and/or hybrids thereof, can be included in the silk/platelet compositions disclosed herein. Those skilled in the art will recognize that the cells listed herein represent an exemplary, not comprehensive, list of cells. Cells can be obtained from donors (allogenic) or from recipients (autologous). Cells can be obtained, as a non-limiting example, by biopsy or other surgical means known to those skilled in the art.

In some aspects, the cell can be a genetically modified cell. A cell can be genetically modified to express and secrete a desired compound, e.g. a bioactive agent, a growth factor, differentiation factor, cytokines, and the like. Methods of genetically modifying cells for expressing and secreting compounds of interest are known in the art and easily adaptable by one of skill in the art.

Differentiated cells that have been reprogrammed into stem cells can also be used.

For example, human skin cells reprogrammed into embryonic stem cells by the transduction of Oct3/4, Sox2, c-Myc and Klf4 (Junying Yu, et. ah, Science, 2007, 318, 1917-1920 and Takahashi K. et. ah, Cell, 2007, 131, 1-12).

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While the invention has been illustrated and described in detail in the foregoing drawings and description, the same is to be considered as illustrative and not restrictive in character, it being understood that only illustrative embodiments thereof have been shown and described and that all changes and modifications that come within the spirit of the invention are desired to be protected. For example, any of the features or functions of any of the embodiments disclosed herein may be incorporated into any of the other embodiments disclosed herein.

### EXAMPLES

### Example 1

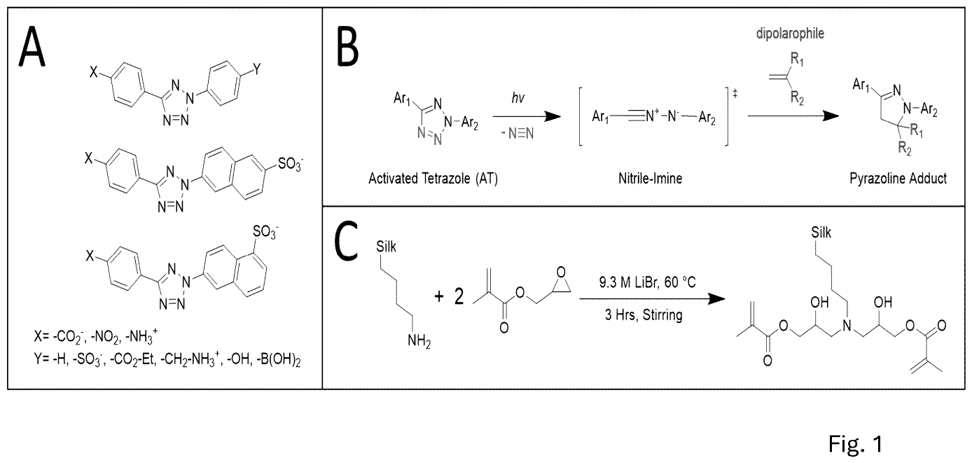

The photoclick tattoo inks are based on the reactions of the 2,5-diaryl-tetrazole (DAT) when exposed to UV-B light in the presence of a dipolarophile. When DAT or a derivative thereof is exposed to 302 nm light, it undergoes a cycloreversion resulting in a reactive nitrile-imine intermediate and molecular nitrogen (FIG. 1B). The large heat of formation of the molecular nitrogen gives this reaction very strong thermodynamic favorability and leads to quantitative yields. The nitrile-imine intermediate is selectively reactive with dipolarophilic groups (typically alkenes) and reacts to produce a 2-5-diphenyl-pyrazoline adduct, which is fluorescently active. Dipolarophiles are biologically rare, thus side-reactions are also rare and give good selectivity when performed in living systems.

Currently, a small library of DATs have been synthesized with varying substitutions (FIG. 1A). These substitutions have a marked impact on the fluorescence of the resulting pyrazoline adducts, and thus enable some degree of control over the final color of emission from the dye. These also enable further functionalization of the photoclick molecules to improve processability and biocompatibility.

In order to produce the fluorescent pyrazoline adduct, the silk must be functionalized with dipolarophilic groups as it does not natively contain any. Methacrylate groups are added to the silk by reacting the fibroin with glycidyl methacrylate (FIG. 1C). This reaction attaches methacrylate groups to the lysine sidechains and n-terminus of silk fibroin molecules. This methacrylated silk (Sil-MA) is then able to participate in the photoclick reaction. This silk has been previously shown to remain biocompatible and bioresorbable post modification. This reaction has been performed successfully and the resultant silk is able to participate in the photoclick reaction.

In order for the tattoos to remain contained within the dermis for useful periods of time, the Sil-MA must be formed into nanoparticles. Silk can be formed into nanoparticles in a number of ways including rapid precipitation⁶, co-flow injection⁷ and microfluidic encapsulation⁸. Currently precipitation using solvents is the simplest method and produces nanoparticles that can undergo the photoclick reaction.

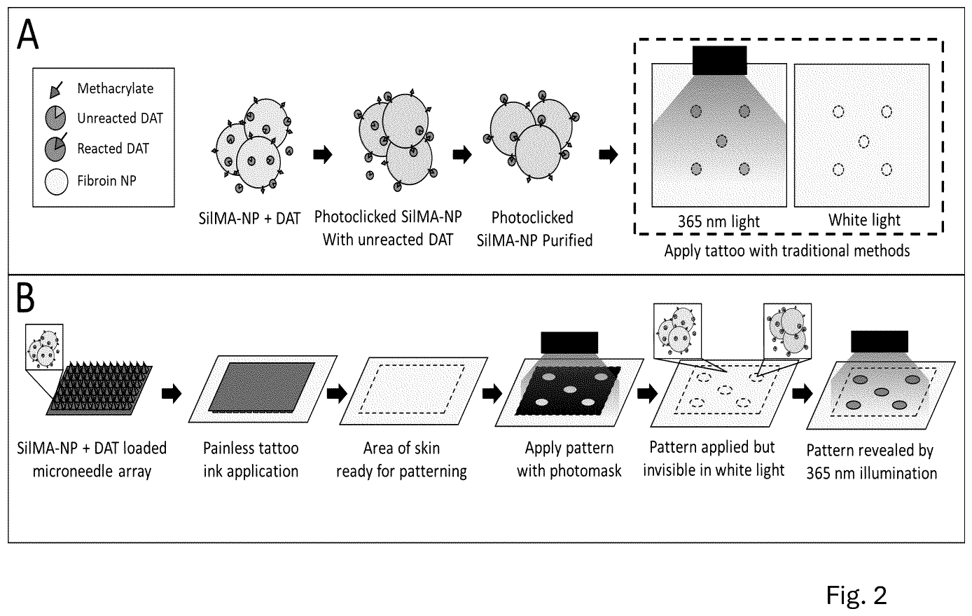

The tattoo inks can be formulated in two general ways; a “traditionally applied” tattoo ink, and a “painless” or “inject-then-write” ink. The traditionally applied tattoo ink behaves as common tattoo inks do and is applied to an individual with an ordinary tattoo gun (FIG. 2A). This ink is formulated by synthesizing Sil-MA nanoparticles and mixing them with the DAT. These are exposed to 302 nm light and the photoclick reaction proceeds in vitro. The nanoparticles are then purified by removing unreacted DAT via solvent washing or dialysis. These nanoparticles are then resuspended in water, filtered, and mixed with any process aids to create the final tattoo ink.

The painless or inject-then-write tattoo inks are formulated differently. Here Sil-MA nanoparticles and unreacted DAT are mixed and applied to a microneedle array (FIG. 2B). The bulk of this microneedle array may be made of silk or another biomaterial. The array is then applied to the individual to inject the unreacted DAT and silk nanoparticles in the general area the mark is desired. Once the ink is in the individual's skin, the exact area is patterned via; 1) exposure to UV-B light through a photomask or 2) via laser stimulation using a multi-photon writing system. Only the areas exposed to the higher energy light will react, leaving the other areas non-fluorescent and invisible under all illumination conditions. The small molecule DAT is expected to be cleared from the area relatively rapidly (<1 week), so there is the potential to write multiple overlapping patterns if sufficient time is allotted between tattoos.

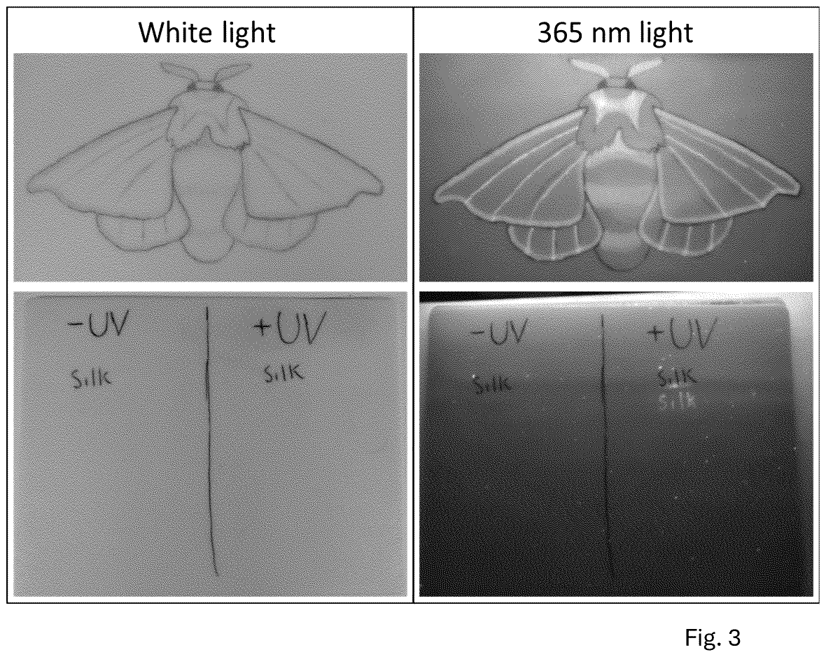

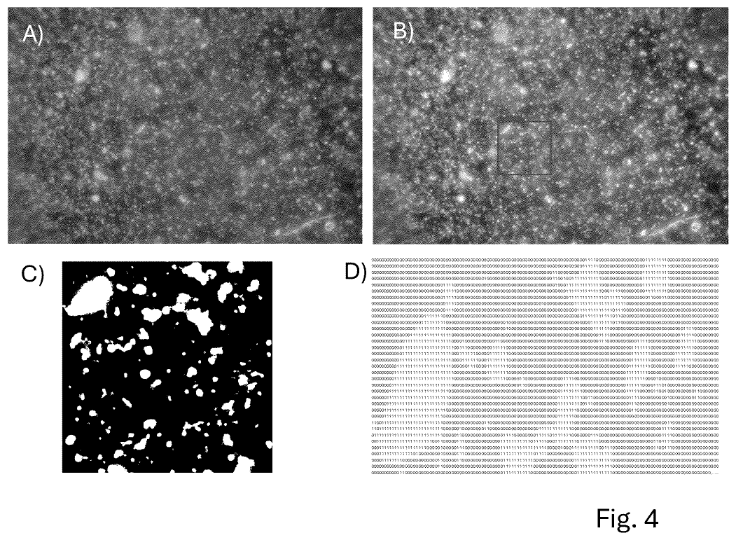

Some demonstrative tattoos are illustrated herein. These tattoos were applied onto simulated silk (silicone) with a commercial tattoo gun. These tattoos can be used for aesthetic purposes, as they allow hidden features of an art piece to be revealed under special illumination (FIG. 3-top). They also demonstrate the feasibility of the inject-then-write dynamic. In FIG. 3-bottom the same tattoo was made on both sides, however only the right-hand side was exposed to UV-B light after tattooing. The ink was able to undergo the photoclick reaction in situ and the word “silk” is revealed.

### Example 2

Silk was methacrylated and microspheres were produced via a co-flow method (Ostrovsky-Snider, N. A. (2023). “The Application of Tetrazole Photoclick Reactions in Patterning Fluorescent Silk Constructs”, Doctoral dissertation, Tufts University, which is incorporated herein in its entirety by reference for all purposes). These microparticles were mixed with diaryltetrazole (DAT) derivative (see below) and exposed to 302 nm light to initiate the photoclick reaction. The particles were then washed, isolated and resuspended in 5% silk fibroin solution. The microparticle laden solution was cast into films and imaged under 356 nm illumination. The green channel of the image was isolated, and turned into a binary code with a 128×128 pixel section yielding a 16 kbit encoded pattern.

### Example 3

### Synthesis and Characterization of 2,5-Diaryltetrazoles

Supporting data for Example 3 can be found in Ostrovsky-Snider, N. A. (2023). “The Application of Tetrazole Photoclick Reactions in Patterning Fluorescent Silk Constructs”, Doctoral dissertation, Tufts University (hereinafter “Ostrovsky-Snider”), which is incorporated herein in its entirety by reference for all purposes. Some of the images provided herein may be viewed more clearly in their original color form within Ostrovsky-Snider.

The core photoreactive species that would perform the photo-mediated conjugation chemistry are based on the 2,5-diaryltetrazole (DAT) species. The synthesis of these reactive groups is both simple and flexible, thus allowing for easy derivatization to improve solubility, reactivity and optical properties. The overall synthetic scheme is laid out in Scheme 1. Beginning with a benzaldehyde derivative, addition of a sulfonylhydrazide will form a sulfonylhydrazone. This sulfonylhydrazone is then dissolved in a basic solvent, in our case pyridine (although literature suggest that aqueous bases may also work) and chilled to −10 C. Addition of a diazonium salt to this solution will cause a 3+2 cycloaddition to occur, forming the tetrazole and ejecting the sulfonic acid as a leaving group. Thus, the DAT is decorated on the 2-phenyl ring by starting with different benzaldehyde derivatives, and on the 5-phenyl position by choosing different diazonium derivatives.

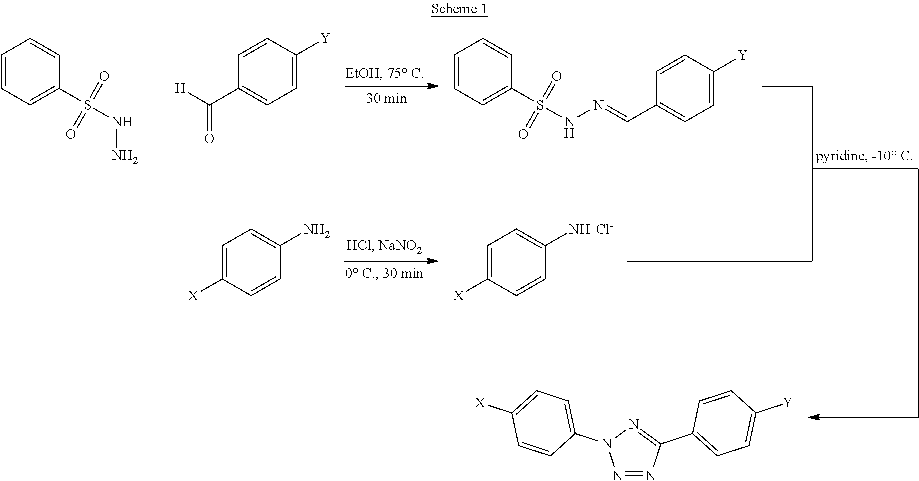

The key difficulty of synthesis lies in the last step, with the reactivity of diazonium salts. These salts readily undergo electrophilic aromatic substitution to form azo dyes. This somewhat limits the selection of derivatives that are possible to synthesize, as electron-rich activating groups on either the benzaldehyde or the aniline will produce undesirable azo-dye side products. Activating groups are not impossible to use (as will be demonstrated later in this chapter) but will severely limit yields and would be best concealed by protecting groups if possible. The diazonium will also react to a limited extent with pyridine to produce azo-dyes as well, however this reaction is slow and does not present a significant limitation.

The derivatives prepared in this process showed useful variation in photoactivation and the resulting fluorescent properties. Derivatives showed variable fluorescent properties based on the electronegativity of their substituents, quenching by the solvent, interactions with the host polymer and the pH of the solvent. The derivatives were characterized in a variety of solvents and pHs and fluorescence titrations were performed to elucidate the pH range of the chromic shifts and possibly suggest a molecular mechanism for the action.

### BACKGROUND

Performing chemistry at the biological interface can be quite difficult due to the abundance and variety of biological molecules that might interfere with reactions by creating side products or disrupting the reaction, and the inherent sensitivity of the biological system to chemical insult. Click chemistries are a class of reactions that aim to solve all these problems simultaneously and thus enable efficient chemistry to interface with biological systems. To achieve this, click reactions are inherently paired reactions, with an energetically ‘loaded’ reactive species that reacts irreversibly with a specific ligand. The name itself arises from the analogy of a spring-loaded buckle. The two halves are made to fit one another and can be pushed together easily, but once mated the spring clicks into place, locking the halves of the buckle together such that pulling them apart is exceptionally difficult.

To build a click-pair you need several things. First, both parts of the pair should be bio-inert, and the resulting adduct should be bio-stable and not degraded by common enzymes or other biological molecules. Secondly you want to ‘spring-load’ the donating portion of the reaction by adding a substantial amount of chemical energy in the form of electronic or steric strain. The release of this thermodynamic loading is what pushes the reaction towards completion and achieves quantitative yields, with Kolb et al. (H. C. Kolb, M. G. Finn, and K. B. Sharpless, “Click Chemistry: Diverse Chemical Function from a Few Good Reactions,” Angew. Chem. Int. Ed. Engl., vol. 40, no. 11, pp. 2004-2021, June 2001, doi: 10.1002/1521-3773(20010601)40:11<2004::aid-anie2004>3.3.co;2-x., which is incorporated herein in its entirety by reference for all purposes) suggesting a driving force of upwards of 20 kcal mol⁻¹. Lastly, the reaction byproducts and ideally any catalytic components should be non-toxic, which would allow this to be done in a living system. Failing that, all byproducts should be easily removable without resorting to chromatographic separations or toxic solvents. A good illustration of these principals is seen in the most familiar click reactions, the copper catalyzed azide-alkyne cycloaddition (CuAAC) reaction (Scheme 2).

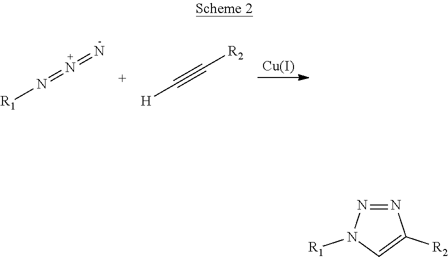

The azide and alkyne groups make up the click pair, and both are tolerated well biologically (despite the exceptional toxicity of azide salts, some organic azides can be sufficiently well tolerated to be considered bioorthogonal). The azide is highly electronically strained, so much so that metal salts of azides are often contact-sensitive explosives. This azide is thus the activated group and is energetically primed to donate this electronic strain to a receiving group that can redistribute the electron density. The terminal alkyne is a biologically inert and a non-natural receiving group that can receive the excess electron density from the azide. Upon the addition of catalytic amounts of copper, the pair is converted into a stable aromatic triazole, relieving the electronic strain and pushing the reaction to quantitative yields. This reaction readily occurs in water, is insensitive to oxygen or other biologically common interferents, and produces no by-products. While the copper catalyst is toxic, it can be easily removed by precipitation. While the toxic copper was tolerable for reactions such as medicinal synthesis, the desire to apply it to a biological environment led to furth exploration in the field to find more bioorthogonal click reactions such as strain promoted azide alkyne cycloaddition, which needs no copper catalyst.

Photoclick chemistries are a subset of reactions that are triggered by the absorption of light instead of requiring a catalyst or occurring spontaneously. The addition of light to the reaction scheme allows for very precise control of the reaction in both space and time, enabling technologies such as cell marking and 3D printing. In addition to the fundamental requirements of click reactions, Fairbanks et al. (B. D. Fairbanks et al., “Photoclick Chemistry: A Bright Idea,” Chem. Rev., vol. 121, no. 12, pp. 6915-6990, June 2021, doi: 10.1021/acs.chemrev.0c01212., which is incorporated herein in its entirety by reference for all purposes) also suggest that for a reaction to be a true “photoclick” reaction it must have rapid reaction times, be triggered by low intensity light, and avoid high-energy activation wavelengths when possible. This class of reactions includes photoinduced azirine-alkenecycloaddition, Hetero-Diels-Alder Cycloaddition and 2,5-diaryltetrazole/alkene cycloaddition.

The 2,5-diaryltetrazole (DAT) alkene photoclick reaction is a fast water/oxygen tolerant reaction that comprises two steps, the first is a photomediated cycloreversion of the tetrazole into a nitrile-imine intermediate (Scheme 3). This unstable intermediate then performs a 3+2 cycloaddition to form a pyrazoline adduct, thus covalently bonding any groups attached to the tetrazole at the 2- and 5-positions with any substituents on the alkene. The DAT group as well as a wide variety of alkenes are all well tolerated in biological environments, the reaction requires no catalyst, has a very large thermodynamic drive from the formation of the molecular nitrogen, and requires no purification, all attributes which make it a true click reaction. On top of this, the reaction is extremely rapid with cycloaddition rate constants as high as 34000±1300 m⁻¹s⁻¹ in biological media, can achieve quantitative yields in equimolar conditions and has a tunable excitation wavelength from UVB to visible blue light. All these criteria together qualify the photoinitated DAT-ene cycloaddition as a true photoclick reaction.

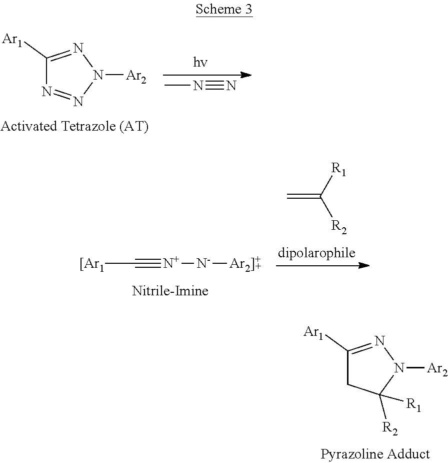

The final product of the DAT-ene photoclick reaction is a 1-3-diaryl-4-substituted pyrazoline, which on top of being biologically stable is also a high-quantum-yield fluorophore with yields near unity in aprotic solvents and up to 0.96 in water at certain pHs. The aryl substituents of the pyrazoline have both inductive and resonance coupling with the Pi-system of the pyrazoline core, thus altering the fluorescent properties of the core. This has been previously demonstrated by An et al. (P. An and Q. Lin, “Sterically shielded tetrazoles for a fluorogenic photoclick reaction: tuning cycloaddition rate and product fluorescence,” Organic & Biomolecular Chemistry, vol. 16, no. 29, pp. 5241-5244, 2018, doi: 10.1039/C8OB01404C., which is incorporated herein in its entirety by reference for all purposes), who were able to tune the emission wavelength of pyrazoline fluorophores across the breadth of the visible spectrum but adding electron donating or withdrawing substituents to the aryl ring on the 1-poisition of the pyrazoline core. This controllability of fluorescent behavior enables the creation of a range of fluorescent dyes that can be used to simultaneously pattern or label regions with spectral independence.

Fluorescence is a photoluminescence process where a material absorbs light at one wavelength and emits it rapidly at a longer wavelength. This is distinct from phosphorescence in that the fluorescent materials only emits the longer wavelength light while being stimulated, whereas a phosphorescent material may emit light for a long period after the exciting light is removed. Fluorescent materials have found a great many utilities in research in medicine, from fluorescent microscopy, tracer dyes, selectively visible markings and many others.

A fluorescent material absorbs light and the electrons in the molecular orbitals are excited from the singlet ground state (S₀) to a singlet excited state (S₀). When the electrons fall back to the ground level the molecule emits a photon that has slightly less energy in a process known as the Stokes shift. The Stokes shift arises from energy loss due to non-radiative energy transfers within the molecule. These are often due to excitation decaying from excited vibrational modes of the ground or excited state. The exact causes of non-radiative relaxation are complicated and difficult to predict, often requiring advanced quantum computational methods such as density functional theory to predict with any degree of accuracy.

The energy in the excited electron is not fully bound to within the molecule and can be transferred to nearby molecules. In the simplest energy transfer the excited state of the initial fluorophore can be transferred to the excited state of a nearby molecule in a process called fluorescence resonance energy transfer (FRET). However for FRET to take place, the molecules must be very spatially close together as the coupling decays with the sixth power of distance. While a fluorophore is in solution, it is constantly very close to another potential acceptor for energy transfer, the solvent itself. If the energy is transferred to the higher vibrational states, they will often decay non-radiatively resulting in the quenching of the fluorescence. The excited state of a red-fluorophore closely matches the energy of vibrational overtones in the O—H bonds, thus fluorophores that are bright red emitters in aprotic solvents like ethyl acetate (EtAc) are efficiently quenched by water. The primary method for circumventing this energy transfer is to trap the fluorescent portion of the molecule in a solvent-inaccessible pocket made from sterically inhibiting groups being placed adjacent to the fluorescent core.

### Methods and Materials

### Isolation, Synthesis, and Purification

### Synthesis of SH-1 (4-[(Phenylsulfonyl)hydrazinylidene]-benzoic acid)

To a 50 mL beaker was added a magnetic stirrer and 10 mL of denatured ethanol, which was brought to a boil. To the boiling ethanol was added 1.501 g (10 mmol) of 4-formylbenzoic acid and stirred vigorously until dissolved (usually 60-120 seconds). Once all solute was dissolved, the heat was turned off and 1.722 g (10 mmol) of benzenesulfonyl hydrazide was added. As stirring continued the solution became light yellow and as it cooled precipitant began to form in solution. The solution was allowed to stir for 2 hours and was cooled to room temperature. To the solution was added 20 mL of deionized water, which formed a white precipitant. The precipitant was collected via vacuum filtration through Whatman number 1 filter paper and washed with ice cold water. The precipitant was allowed to fully dry on a watch glass and then was transferred to a storage bottle and kept in a dark cabinet at room temperature. ¹H NMR (DMSO-d6, 500 MHz) δ 13.1 (s, 1H, OH), δ 11.78 (s, 1H, NH), δ 7.99 (s, 1H, CH), δ 7.94 (d, 2H, CH, J=8.3 Hz), δ 7.9 (d, 2H, CH, J=7.2 Hz), δ 7.67 (t, 3H, CH, J=6.5 Hz), δ 7.63 (t, 2H, CH, J=7.4 Hz) HRMS (ESI-TOF) m/z: [M−H]− Calcd for C₁₄H₁₁N₂O₄S₁ 303.0440; Found 303.0440

### Synthesis of SH-2 (4-[(Phenylsulfonyl)hydrazinylidene]-nitrobenzene)

To a 50 mL beaker was added a magnetic stirrer and 10 mL of denatured ethanol, which was brought to a boil. To the boiling ethanol was added 1.511 g (10 mmol) of 4-nitrobenzaldehyde and stirred vigorously until dissolved (usually 60-120 seconds). Once all solute was dissolved, the heat was turned off and 1.722 g (10 mmol) of benzenesulfonyl hydrazide was added. As stirring continued the solution became intensely yellow and as it cooled precipitant began to form in solution. The solution was allowed to stir for 2 hours and was cooled to room temperature. To the solution was added 20 mL of deionized water, which formed a yellow precipitant. The precipitant was collected via vacuum filtration through Whatman number 1 filter paper and washed with ice cold water. The precipitant was allowed to fully dry on a watch glass and then was transferred to a storage bottle and kept in a dark cabinet at room temperature. ¹H NMR (DMSO-d6, 500 MHz) δ 12.01 (s, 1H, NH), δ 8.24 (d, 2H, CH, J=8.8 Hz), δ 8.04 (s, 1H, CH), δ 7.91 (d, 2H, CH, J=7.2 Hz), δ 7.83 (d, 2H, CH, J=8.8 Hz), δ 7.68 (t, 1H, CH, J=7.3 Hz), δ 7.63 (t, 2H, CH, J=7.4 Hz) HRMS (ESI-TOF) m/z: [M−H]− Calcd for C₁₃H₁₀N₃O₄S₁ 304.0392; Found 304.0396

### Synthesis of SH-3 (3-[(Phenylsulfonyl)hydrazinylidene]-nitrobenzene)

To a 50 mL beaker was added a magnetic stirrer and 10 mL of denatured ethanol, which was brought to a boil. To the boiling ethanol was added 1.511 g (10 mmol) of 3-nitrobenzaldehyde and stirred vigorously until dissolved (usually 60-120 seconds). Once all solute was dissolved, the heat was turned off and 1.722 g (10 mmol) of benzenesulfonyl hydrazide was added. As stirring continued the solution became intensely yellow and as it cooled precipitant began to form in solution. The solution was allowed to stir for 2 hours and was cooled to room temperature. To the solution was added 20 mL of deionized water, which formed a yellow precipitant. The precipitant was collected via vacuum filtration through Whatman number 1 filter paper and washed with ice cold water. The precipitant was allowed to fully dry on a watch glass and then was transferred to a storage bottle and kept in a dark cabinet at room temperature. ¹H NMR (DMSO-d6, 500 MHz) δ 11.91 (s, 1H, NH), δ 8.37 (s, 1H, CH), δ 8.23 (q, 1H, CH, J=3.2 Hz), δ 8.08 (s, 1H, CH), δ 8.02 (d, 1H, CH, J=7.7 Hz), δ 7.91 (d, 2H, CH, J=7.2 Hz), δ 7.69 (t, 1H, CH, J=7.9 Hz), δ 7.67 (d, 1H, CH, J=7.1 Hz), δ 7.63 (t, 2H, CH, J=7.3 Hz) HRMS (ESI-TOF) m/z: [M−H]− Calcd for C₁₃H₁₀N₃O₄S₁ 304.0392; Found 304.0388

### Synthesis of SH-4 (4-[(Phenylsulfonyl)hydrazinylidene]-phenol)

To a 50 mL beaker was added a magnetic stirrer and 10 mL of denatured ethanol, which was brought to a boil. To the boiling ethanol was added 1.221 g (10 mmol) of 4-hydroxybenzaldehyde and stirred vigorously until dissolved (usually 60-120 seconds). Once all solute was dissolved, the heat was turned off and 1.722 g (10 mmol) of benzenesulfonyl hydrazide was added. As stirring continued the solution became pink and as it cooled precipitant began to form in solution. The solution was allowed to stir for 2 hours and was cooled to room temperature. To the solution was added 20 mL of deionized water, which formed a pink precipitant. The precipitant was collected via vacuum filtration through Whatman number 1 filter paper and washed with ice cold water. The precipitant was allowed to fully dry on a watch glass and then was transferred to a storage bottle and kept in a dark cabinet at room temperature. ¹H NMR (DMSO-d6, 500 MHz) δ 11.22 (s, 1H, NH), δ 9.9 (s, 1H, OH), δ 7.87 (d, 2H, CH, J=7.1 Hz), δ 7.82 (s, 1H, CH), δ 7.66 (t, 1H, CH, J=7.3 Hz), δ 7.61 (t, 2H, CH, J=7.3 Hz), δ 7.39 (d, 2H, CH, J=8.6 Hz), δ 6.77 (d, 2H, CH, J=8.6 Hz) HRMS (ESI-TOF) m/z: [M−H]− Calcd for C₁₃H₁₁N₂O₃S₁ 275.0490; Found 275.0488

### Synthesis of DAT 1 (2-(4-phenyl)-5-(4-carboxyphenyl)-2H-tetrazole)

To a 4-dram scintillation vial was added a magnetic stirrer, 6 mL of pyridine and 304 mg (1 mmol) of SH-1. The solution was stirred until all solute was dissolved, then placed into an ice-brine bath to cool for at least 15 minutes. To a small centrifuge tube was added 800 μL of denatured ethanol, 800 μL of deionized water, 260 μL of concentrated hydrochloric acid and 93 μL of aniline. The solution was mixed by repeated inversion and placed into the ice-brine bath to cool for 15 minutes. To form the diazonium, 250 μL of 4 M sodium nitrite solution was added to the centrifuge tube, which was quickly inverted to mix then replaced in the ice-brine bath. If you observe the generation of gas upon the addition of the sodium nitrite, the aniline solution was not allowed to cool sufficiently before nitrite addition and must be remade from scratch. The diazonium solution was added dropwise to the pyridine solution, with bursts of red color accompanying each drop followed by a reversion to a light-yellow color. As the reaction progressed the solution became a clear deep red. After all the diazonium solution was added, the solution was allowed to mix for a further 30 minutes in the ice-brine bath. Afterwards it was removed from the bath, capped, and allowed to stir further overnight at room temperature. The product was extracted 3 times with water/ethyl acetate and the organic layers were combined and washed with saturated brine solution. The organic layer was then dried over excess sodium sulfate and afterward the solvent was removed by vacuum-assisted rotary evaporation yielding a flaky, burgundy-colored solid. The solid was recrystallized from boiling methanol and allowed to cool to room temperature over 1 day and to −20 C over the next day. Small (˜1 mm in length) needle-like crystals formed that appeared red in bulk but were yellow when viewed under a microscope. These crystals were isolated by vacuum filtration on Whatman number 1 paper, then washed with a small amount of cold (−20 C) methanol and dried under ambient conditions. The crystals were stored in an opaque, sealed bottle at room temperature until use. ¹H NMR (DMSO-d6, 500 MHz) δ 8.32 (d, 2H, CH, J=8.4 Hz), δ 8.19 (t, 4H, CH, J=8.5 Hz), δ 7.73 (t, 2H, CH, J=7.7 Hz), δ 7.66 (t, 1H, CH, J=7.4 Hz) HRMS (ESI-TOF) m/z: [M−H]− Calcd for C₁₄H₉N₄O₂ 265.0726; Found 265.1477

### Synthesis of DAT 2 (2-(4-(ethoxycarbonyl)-phenyl)-5-(4-carboxyphenyl)-2H-tetrazole)

To a 4-dram scintillation vial was added a magnetic stirrer, 6 mL of pyridine and 304 mg (1 mmol) of SH-1. The solution was stirred until all solute was dissolved, then placed into an ice-brine bath to cool for at least 15 minutes. To a small centrifuge tube was added 800 μL of denatured ethanol, 800 μL of deionized water, 260 μL of concentrated hydrochloric acid and 165 mg of benzocaine. The solution was mixed by repeated inversion and placed into the ice-brine bath to cool for 15 minutes. To form the diazonium, 250 μL of 4 M sodium nitrite solution was added to the centrifuge tube, which was quickly inverted to mix then replaced in the ice-brine bath. If you observe the generation of gas upon the addition of the sodium nitrite, the aniline solution was not allowed to cool sufficiently before nitrite addition and must be remade from scratch. The diazonium solution was added dropwise to the pyridine solution, with bursts of red color accompanying each drop followed by a reversion to a light-yellow color. As the reaction progressed the solution became a clear deep red. After all the diazonium solution was added, the solution was allowed to mix for a further 30 minutes in the ice-brine bath. Afterwards it was removed from the bath, capped, and allowed to stir further overnight at room temperature. The product was extracted 3 times with water/ethyl acetate and the organic layers were combined and washed with saturated brine solution. The organic layer was then dried over excess sodium sulfate and afterward the solvent was removed by vacuum-assisted rotary evaporation yielding a lightly red powder. The powder was redissolved in methanol and purified by reverse phase flash chromatography using a methanol/water mobile phase and C-18 modified silica gel stationary phase. Mobile phase was a linear ramp from 100% methanol to 10% methanol in water over 15 minutes at a flow rate of 25 mL per minute. Fractions containing the product were consolidated and dried under vacuum to yield a white powder. ¹H NMR (DMSO-d6, 500 MHz) δ 8.35 (d, 2H, CH, J=8.2 Hz), δ 8.31 (d, 2H, CH, J=7.9 Hz), δ 8.26 (d, 2H, CH, J=8.2 Hz), δ 8.17 (d, 2H, CH, J=7.8 Hz), δ 4.38 (q, 2H, CH2, J=6.7 Hz), δ 1.37 (t, 3H, CH2, J=6.8 Hz) HRMS (ESI-TOF) m/z: [M−H]− Calcd for C₁₇H₁₃N₄O₄ 337.0937; Found 337.0956

### Synthesis of DAT 3 (2-(4-sulfophenyl)-5-(4-carboxyphenyl)-2H-tetrazole)

To a 4-dram scintillation vial was added a magnetic stirrer, 6 mL of pyridine and 304 mg (1 mmol) of SH-1. The solution was stirred until all solute was dissolved, then placed into an ice-brine bath to cool for at least 15 minutes. To a small centrifuge tube was added 10 mL of deionized water, 1.72 g of para-toluene sulfonic acid and 173 mg of sulfamic acid. The solution was mixed extensively by alternating vortexing and bath sonication over the course of 30 minutes then placed into the ice-brine bath to cool for 15 minutes. To form the diazonium, 250 μL of 4 M sodium nitrite solution was added to the centrifuge tube, which was quickly inverted to mix then replaced in the ice-brine bath. If you observe the generation of gas upon the addition of the sodium nitrite, the aniline solution was not allowed to cool sufficiently before nitrite addition and must be remade from scratch. The diazonium solution was added dropwise to the pyridine solution, with bursts of red color accompanying each drop followed by a reversion to a light-yellow color. As the reaction progressed the solution became a clear deep red. After all the diazonium solution was added, the solution was allowed to mix for a further 30 minutes in the ice-brine bath. Afterwards it was removed from the bath, capped, and allowed to stir further overnight at room temperature. The crude product was dried under vacuum the redissolved in deionized water. The product was precipitated by the addition of acetic acid and the precipitant was collected via vacuum filtration. Crystallization was attempted from boiling water, however precipitant formed rapidly upon cooling such that no crystals were able to grow. This precipitant was captured by vacuum filtration and washed with ice cold water. The product was dried under ambient conditions then stored in a dark vial at room temperature. ¹H NMR (D2O, 500 MHz) δ 8.27 (d, 2H, CH, J=8.4 Hz), δ 8.19 (d, 2H, CH, J=8.1 Hz), δ 8.02 (d, 2H, CH, J=8.5 Hz), δ 7.99 (d, 2H, CH, J=8 Hz) HRMS (ESI-TOF) m/z: [M−H]− Calcd for C₁₄H₉N₄O₅S₁ 345.0294; Found 344.9796

### Synthesis of DAT 4 (2-(4-hydroxyphenyl)-5-(4-carboxyphenyl)-2H-tetrazole)

To a 4-dram scintillation vial was added a magnetic stirrer, 6 mL of pyridine and 304 mg (1 mmol) of SH-1. The solution was stirred until all solute was dissolved, then placed into an ice-brine bath to cool for at least 15 minutes. To a small centrifuge tube was added 800 μL of denatured ethanol, 800 μL of deionized water, 260 μL of concentrated hydrochloric acid and 109 mg of aminophenol. The solution was mixed by repeated inversion and placed into the ice-brine bath to cool for 15 minutes. To form the diazonium, 250 μL of 4 M sodium nitrite solution was added to the centrifuge tube, which was quickly inverted to mix. The diazonium solution was added immediately to the pyridine solution, and the solution instantly became a deep red color. After all the diazonium solution was added, the solution was allowed to mix for a further 30 minutes in the ice-brine bath. Afterwards it was removed from the bath, capped, and allowed to stir further overnight at room temperature. The product was extracted 3 times with water/ethyl acetate and the organic layers were combined and washed with saturated brine solution. The organic layer was then dried over excess sodium sulfate and afterward the solvent was removed by vacuum-assisted rotary evaporation yielding a viscous crimson oil. The oil was recrystallized from boiling methanol and allowed to cool to room temperature over 1 day and to −20 C over the next day. Irregular red crystals were formed after two days. These crystals were isolated by vacuum filtration on Whatman number 1 paper, then washed with a small amount of cold (−20 C) methanol and dried under ambient conditions. The crystals were stored in an opaque, sealed bottle at room temperature until use. ¹H NMR (CDCl3, 500 MHz) δ 13.26 (s, 1H, OH), δ 10.28 (s, 1H, OH), δ 8.28 (d, 2H, CH, J=8.4 Hz), δ 8.16 (d, 2H, CH, J=8.3 Hz), δ 7.98 (d, 2H, CH, J=8.9 Hz), δ 7.04 (d, 2H, CH, J=8.9 Hz) HRMS (ESI-TOF) m/z: [M−H]− Calcd for C₁₄H₉N₄O₃ 281.0675; Found 281.0689

### Synthesis of DAT 5 (2-(4-boronophenyl)-5-(4-carboxyphenyl)-2H-tetrazole)

To a 4-dram scintillation vial was added a magnetic stirrer, 6 mL of pyridine and 304 mg (1 mmol) of SH-1. The solution was stirred until all solute was dissolved, then placed into an ice-brine bath to cool for at least 15 minutes. To a small centrifuge tube was added 800 μL of denatured ethanol, 800 μL of deionized water, 260 μL of concentrated hydrochloric acid and 173 mg of 4-aminophenylboronic acid. The solution was mixed by repeated inversion and placed into the ice-brine bath to cool for 15 minutes. To form the diazonium, 250 μL of 4 M sodium nitrite solution was added to the centrifuge tube, which was quickly inverted to mix then replaced in the ice-brine bath. If you observe the generation of gas upon the addition of the sodium nitrite, the aniline solution was not allowed to cool sufficiently before nitrite addition and must be remade from scratch. The diazonium solution was added dropwise to the pyridine solution, with bursts of red color accompanying each drop followed by a reversion to a light-yellow color. As the reaction progressed the solution became a clear deep red. After all the diazonium solution was added, the solution was allowed to mix for a further 30 minutes in the ice-brine bath. Afterwards it was removed from the bath, capped, and allowed to stir further overnight at room temperature. The product was extracted 3 times with water/ethyl acetate and the organic layers were combined and washed with saturated brine solution. The organic layer was then dried over excess sodium sulfate and afterward the solvent was removed by vacuum-assisted rotary evaporation yielding brown solid. The solid was recrystallized from boiling methanol and allowed to cool to room temperature over 1 day and to −20 C over the next day. Irregular brown crystals form and these crystals were isolated by vacuum filtration on Whatman number 1 paper, then washed with a small amount of cold (−20 C) methanol and dried under ambient conditions. The crystals were stored in an opaque, sealed bottle at room temperature until use. ¹H NMR (DMSO-d6, 500 MHz) δ 13.28 (s, 1H, OH), δ 8.4 (s, 2H, OH), δ 8.32 (d, 2H, CH, J=8.2 Hz), δ 8.18 (d, 2H, CH, J=5.2 Hz), δ 8.17 (d, 2H, CH, J=5.3 Hz), δ 8.09 (d, 2H, CH, J=8.5 Hz) HRMS (ESI-TOF) m/z: [M−H]− Calcd for C₁₄H₁₀B₁N₄O₄ 309.0795; Found 309.0810

### Synthesis of DAT 6 (5-(4-nitrophenyl)-2-phenyl-2H-tetrazole)

To a 4-dram scintillation vial was added a magnetic stirrer, 6 mL of pyridine and 305 mg (1 mmol) of SH-2. The solution instantly became bright orange and was stirred until all solute was dissolved, then placed into an ice-brine bath to cool for at least 15 minutes. To a small centrifuge tube was added 800 μL of denatured ethanol, 800 μL of deionized water, 260 μL of concentrated hydrochloric acid and 93 μL of aniline. The solution was mixed by repeated inversion and placed into the ice-brine bath to cool for 15 minutes. To form the diazonium, 250 μL of 4 M sodium nitrite solution was added to the centrifuge tube, which was quickly inverted to mix then replaced in the ice-brine bath. If you observe the generation of gas upon the addition of the sodium nitrite, the aniline solution was not allowed to cool sufficiently before nitrite addition and must be remade from scratch. The diazonium solution was added dropwise to the pyridine solution, with bursts of red color accompanying each drop followed by a reversion to a vivid orange color. As the reaction progressed the solution became a clear deep red. After all the diazonium solution was added, the solution was allowed to mix for a further 30 minutes in the ice-brine bath. Afterwards it was removed from the bath, capped, and allowed to stir further overnight at room temperature. The product had precipitated overnight and was isolated by vacuum filtration, yielding a light pink solid. The product was washed by a small amount of ethyl acetate until all the red/pink coloration had been removed. The product was then dried at room temperature and stored in a opaque, sealed bottle until use. ¹H NMR (DMSO-d6, 500 MHz) δ 8.46 (q, 4H, CH, J=8 Hz), δ 8.2 (d, 2H, CH, J=7.9 Hz), δ 7.73 (t, 2H, CH, J=7.6 Hz), δ 7.67 (t, 2H, CH, J=7.4 Hz) HRMS (ESI-TOF) m/z: [M−H]− Calcd for C₁₃H₇N₅O₂ 265.0600; Found 265.1485

### Synthesis of DAT 7 (5-(3-nitrophenyl)-2-phenyl-2H-tetrazole)

To a 4-dram scintillation vial was added a magnetic stirrer, 6 mL of pyridine and 305 mg (1 mmol) of SH-3. The solution instantly became bright orange and was stirred until all solute was dissolved, then placed into an ice-brine bath to cool for at least 15 minutes. To a small centrifuge tube was added 800 μL of denatured ethanol, 800 μL of deionized water, 260 μL of concentrated hydrochloric acid and 93 μL of aniline. The solution was mixed by repeated inversion and placed into the ice-brine bath to cool for 15 minutes. To form the diazonium, 250 μL of 4 M sodium nitrite solution was added to the centrifuge tube, which was quickly inverted to mix then replaced in the ice-brine bath. If you observe the generation of gas upon the addition of the sodium nitrite, the aniline solution was not allowed to cool sufficiently before nitrite addition and must be remade from scratch. The diazonium solution was added dropwise to the pyridine solution, with bursts of red color accompanying each drop followed by a reversion to a vivid orange color. As the reaction progressed the solution became a clear deep red. After all the diazonium solution was added, the solution was allowed to mix for a further 30 minutes in the ice-brine bath. Afterwards it was removed from the bath, capped, and allowed to stir further overnight at room temperature. The product had precipitated overnight and was isolated by vacuum filtration, yielding a light pink solid. The product was washed by a small amount of ethyl acetate until all the red/pink coloration had been removed. The product was then dried at room temperature and stored in a opaque, sealed bottle until use. ¹H NMR (DMSO-d6, 500 MHz) δ 8.88 (s, 1H, CH), δ 8.61 (d, 1H, CH, J=7.8 Hz), δ 8.46 (q, 1H, CH, J=3.2 Hz), δ 8.21 (d, 1H, CH, J=7.8 Hz), δ 7.94 (t, 1H, CH, J=8 Hz), δ 7.73 (t, 2H, CH, J=7.6 Hz), δ 7.66 (t, 1H, CH, J=7.4 Hz) HRMS (ESI-TOF) m/z: [M−H]− Calcd for C₁₃H₇N₅O₂ 265.0600; Found 265.1483

### Synthesis of DAT 8 (5-(4-aminophenyl)-2-phenyl-2H-tetrazole)

To a round bottom flask was added a magnetic stirrer, 5 mL of ethanol, 1 mL of DI water, 50 μL of concentrated hydrochloric acid, 267 mg of DAT-6 and 681 mg of Iron (0) powder. The flask was fitted with an air-cooled reflux column then heated to boiling. The reaction was monitored by TLC and removed from the heat once the reactants had been fully consumed, generally this took about 1 hour. The reaction mixture was allowed to cool then the supernatant was decanted, and the remaining solids extracted by 3 washes with ethyl acetate. The supernatant and extraction solutions were combined filtered through a 0.22 μm PVDF filter to remove the residual iron particles. The combined organic layers were washed with saturated brine, dried over sodium sulfate, then the remaining solvent was removed under vacuum resulting in a canary yellow powder. This powder was then purified by normal phase flash chromatography using an isocratic flow of 2:1 hexanes:ethyl acetate as the mobile phase and silica gel as the stationary phase. The solvent was removed under vacuum, yielding a bright yellow powder, which was stored in a sealed opaque vial at room temperature. ¹H NMR (DMSO-d6, 500 MHz) δ 8.13 (d, 2H, CH, J=7.8 Hz), δ 7.84 (d, 2H, CH, J=8.6 Hz), δ 7.68 (t, 2H, CH, J=7.8 Hz), δ 7.6 (t, 1H, CH, J=7.4 Hz), δ 6.72 (d, 2H, CH, J=8.6 Hz), δ 5.74 (s, 2H, NH2) HRMS (ESI-TOF) m/z: [M+H]+ Calcd for C₁₃H₁₂N₅ 238.1093; Found 238.1086

### Synthesis of DAT 9 (5-(3-aminophenyl)-2-phenyl-2H-tetrazole)

To a round bottom flask was added a magnetic stirrer, 5 mL of ethanol, 1 mL of DI water, 50 μL of concentrated hydrochloric acid, 267 mg of DAT-7 and 681 mg of Iron (0) powder. The flask was fitted with an air-cooled reflux column then heated to boiling. The reaction was monitored by TLC and removed from the heat once the reactants had been fully consumed, generally this took about 1 hour. The reaction mixture was allowed to cool then the supernatant was decanted, and the remaining solids extracted by 3 washes with ethyl acetate. The supernatant and extraction solutions were combined filtered through a 0.22 μm PVDF filter to remove the residual iron particles. The combined organic layers were washed with saturated brine, dried over sodium sulfate, then the remaining solvent was removed under vacuum resulting in a canary yellow powder. This powder was then purified by normal phase flash chromatography using an isocratic flow of 2:1 hexanes:ethyl acetate as the mobile phase and silica gel as the stationary phase. The solvent was removed under vacuum, yielding a bright yellow powder, which was stored in a sealed opaque vial at room temperature. ¹H NMR (DMSO-d6, 500 MHz) δ 8.15 (d, 2H, CH, J=7.6 Hz), δ 7.71 (t, 2H, CH, J=7.8 Hz), δ 7.63 (t, 1H, CH, J=7.4 Hz), δ 7.43 (s, 1H, CH), δ 7.31 (d, 1H, CH, J=7.6 Hz), δ 7.23 (t, 1H, CH, J=7.8 Hz), δ 6.75 (q, 1H, CH, J=3.1 Hz), δ 5.46 (s, 2H, NH2) HRMS (ESI-TOF) m/z: [M+H]+ Calcd for C₁H₁₂N₅ 238.1093; Found 238.1091

### Synthesis of DAT 10 (5-(4-hydroxyphenyl)-2-phenyl-2H-tetrazole)

To a 4-dram scintillation vial was added a magnetic stirrer, 6 mL of pyridine and 276 mg (1 mmol) of SH-4. The solution was stirred until all solute was dissolved, then placed into an ice-brine bath to cool for at least 15 minutes. To a small centrifuge tube was added 800 μL of denatured ethanol, 800 μL of deionized water, 260 μL of concentrated hydrochloric acid and 93 μL of aniline. The solution was mixed by repeated inversion and placed into the ice-brine bath to cool for 15 minutes. To form the diazonium, 250 μL of 4 M sodium nitrite solution was added to the centrifuge tube, which was quickly inverted to mix then replaced in the ice-brine bath. If you observe the generation of gas upon the addition of the sodium nitrite, the aniline solution was not allowed to cool sufficiently before nitrite addition and must be remade from scratch. The diazonium solution was added dropwise to the pyridine solution, with bursts of red color accompanying each drop followed by a reversion to a light-yellow color. As the reaction progressed the solution became a clear deep red. After all the diazonium solution was added, the solution was allowed to mix for a further 30 minutes in the ice-brine bath. Afterwards it was removed from the bath, capped, and allowed to stir further overnight at room temperature. The product was extracted 3 times with water/ethyl acetate and the organic layers were combined and washed with saturated brine solution. The organic layer was then dried over excess sodium sulfate and afterward the solvent was removed by vacuum-assisted rotary evaporation yielding a pink solid. The solid was recrystallized from boiling methanol and allowed to cool to room temperature over 1 day and to −20 C over the next day, allowing irregular red crystals to form. These crystals were isolated by vacuum filtration on Whatman number 1 paper, then washed with a small amount of cold (−20 C) methanol and dried under ambient conditions. The crystals were stored in an opaque, sealed bottle at room temperature until use. ¹H NMR (DMSO-d6, 500 MHz) δ 10.1 (s, 1H, OH), δ 8.15 (d, 2H, CH, J=7.8 Hz), δ 8.01 (d, 2H, CH, J=8.6 Hz), δ 7.7 (t, 2H, CH, J=7.8 Hz), δ 7.62 (t, 1H, CH, J=7.4 Hz), δ 6.98 (d, 2H, CH, J=8.6 Hz) HRMS (ESI-TOF) m/z: [M−H]− Calcd for C₁₃H₉N₄O₁ 237.0776; Found 237.0789

### Chemical Analysis

### pH Measurement

Sample pH was measured with a Metler Toledo SevenMulti pH meter with a ROSS Ultra pHATC triode probe. Prior to measurement each day the pH meter was calibrated with fresh pH standards (Supelco 1.99005). Separate probes were used for measuring silk fibroin and small molecule or buffer solution.

### Ultraviolet and Visible Light (UV-Vis) Spectroscopy

Spectroscopy was performed using a Synergy HT plate reader in with sample Corning Costar UV-transparent 96 well plates (Corning 3635). Initially samples were diluted to 1 mg/mL in either IPA or DIW and a dilution curve was made with each step reducing the concentration by half. The concentration chosen for measurement was the most concentrated sample that had the peak of interest entirely below 1.0 absorption units. When an appropriate concentration was found 100 μL of sample was then loaded into each well with samples loaded in triplicate. If the standard deviation between replicates exceeded 0.05 AU the measurement was repeated with fresh samples.

### Fourier Transform Infrared (FTIR) Spectroscopy

FTIR spectroscopy was performed using a Bruker Invenio spectrometer with a diamond attenuated total reflectance analysis module. Spectra were collected from 4000-400 cm⁻¹ with a 1 cm⁻¹ resolution and 64 averaged scans. Spectra were processed using Bruker Opus software, which consisted of 1 iteration of rubber band baseline correction.

### Nuclear Magnetic Resonance (NMR) Spectroscopy

NMR spectroscopy was performed on a Bruker Avance III 500 MHz spectrometer. Before analysis, small molecule samples were dried under vacuum, while silk fibroin samples were lyophilized, then samples were dissolved in either deuterium oxide with 0.05% 3-(trimethylsilyl)propionic-2,2,3,3-d4 (TMSP-d4) acid or dimethyl sulfoxide-d6 with 0.03% tetramethylsilane (TMS). Peaks were integrated and 0 ppm was calibrated with peaks of either the TMS or TMSP-d4 peak using Bruker TopSpin software.

### Fluorescence Spectroscopy

Fluorophores bound to either silk or PEG-DA were generated by dissolving a DAT precursor to a final concentration of 100 μM in water for silk-bound fluorophores and either DIW, IPA or ethyl acetate (EtAc) for PEG-DA-bound fluorophores. These were reacted by exposure to 302 nm light (specs) for 10 minutes under constant stirring. To an opaque black 96-well plate (Corning 3915) was added 3 separate 100 μL aliquots of fluorophore solution. These were measured by a Molecular Devices SpectraMax M2 plate reader, exciting at either 300, 365 or 405 nm and recording spectra from 25 nm below the excitation wavelength down to 650 nm with a 5 nm resolution. The three samples were averaged, and emission peaks were found by first derivative peak detection.

### Fluorescence Titration

A 100 mL beaker was placed on a stir plate and filled with 50 mL of either deionized water or isopropyl alcohol and a magnetic stirrer. Fluorescent pyrazoline bound to PEG was added to a final concentration of 1 mg/mL and stirred until homogenous. An Ocean Optics SR fiber spectrometer was attached to a UV-Vis optical fiber (200-800 nm, 200 μm core diameter) and the fiber was mounted such that it was normal to the surface of the liquid. A 365 nm LED was placed at such that it illuminated the beaker from the side, perpendicular to the orientation of the fiber. If deionized water was used as the solvent, a pH probe was immersed in the liquid as well. The LED was switched on and the spectrometer exposure time was adjusted until the fluorescent peak reached at least 1000 counts or 3 seconds of exposure, whichever occurred first. During capture 5 exposures were averaged and smoothed by a 9-point moving average. 1 M potassium hydroxide dissolved in either water or isopropyl alcohol was added in 5 μL aliquots until the pH was at least 10 and the fluorescent response of the fluorophore remained constant for 3 consecutive additions. An initial spectrum was acquired then a 5 μL aliquot of 1 M p-toluene sulfonic acid was added and a spectrum was acquired. This was repeated until a pH of 3 had been reached and the fluorescent response was constant for the last 3 additions.

### Fluorescence Kinetics Measurements

Solutions of PEG-DA (10 mg/mL) and DAT (0.1 mg/mL) in IPA were reacted for 5-960 seconds of exposure to 254 or 302 nm light using a Labortechnik UVLMS-38 8 Watt UV multiwavelength lamp. To an opaque black 96-well plate (Corning 3915) was added 100 μL aliquots of fluorophore solution. These were measured using a Synergy HT plate reader with fluorescence measured using excitation at 360 or 485 nm and emission measured at 460, 528, and 590 nm, with 460 nm emission excluded during 485 nm excitation. Kinetics calculations were made with the excitation/emission filter pair that measured the highest emission intensity.

### Results and Discussion

### Synthesis of 2,5-diaryltetrazole Derivatives

Various derivatives were created for the purpose of installing reactive sites for subsequent conjugation and examining the effects the substituents had on the excitation wavelength for the photoclick reaction and the fluorescence of the post-click pyrazoline. To allow for improved conjugation, a benzylic amine was added at on the 2-phenyl ring, in either the meta or para position relative to the tetrazole. The 5-phenyl ring was also replaced with a naphthyl group to shift the excitation wavelength of the photoclick process to longer wavelengths. Lastly the 5 phenyl position had various electron donating and withdrawing substituents added to try to alter the fluorescent emission of the resultant pyrazoline adducts.

The first phase of synthesis is the creation of the sulfonylhydrazone. This is done by heating ethanol to boiling, then adding the desired benzaldehyde derivative to the hot ethanol (Scheme 4). This is stirred until the benzaldehyde is completely dissolved then an equimolar amount of benzenesulfonyl hydrazide is added and the solution is removed from the heat. The sulfonylhydrazone starts to form very rapidly. For highly soluble hydrazones (such as those from carboxybenzaldehyde) remain soluble and you may see a slight yellow color begin to develop in solution. In the case of poorly soluble benzaldehydes (such as nitro derivatives) the solution will begin to shimmer as what are presumably microscopic crystals begin to form in suspension. The solution is stirred continuously while reacting and allowed to cool to room temperature. Once at room temperature the ethanol solution is added to twice its volume of deionized water and the hydrazone will precipitate readily. This can be isolated by vacuum filtration followed by washing with cold deionized water. If higher purity is desired, the sulfonylhydrazone can be readily recrystallized in hot ethanol, but this is not typically needed for this synthesis.

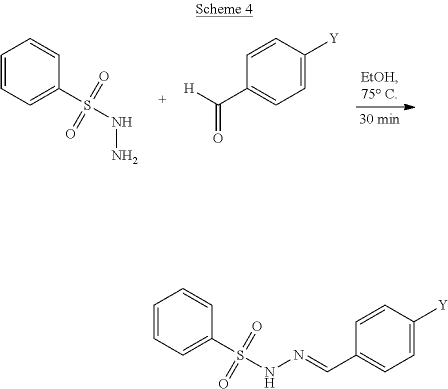

The production of the sulfonylhydrazone can be easily confirmed by infrared spectroscopy. The S══O stretches are clearly evident and are shifted minutely compared to the peaks in the starting material (data can be provided to an examiner, as needed, but is described in the next paragraph). Also evident is the loss of the carbonyl peak from the starting material, as this bond is now occupied by the imide group. Lastly the nitro stretches are also carried over from the aldehyde starting material. Further IR spectra can be found in Appendix 1 (A1.1) of Ostrovsky-Snider as can liquid chromatography-mass spectrometry (LC-MS) analysis (A1.2), and nuclear magnetic resonance spectra (A1.3) of compounds SH-1-4.

FT-IR spectra were prepared for reactants for SH-3 and the resulting sulfonylhydrazone. The spectrum for 4-nitrobenzadehyde included characteristic peaks for the nitro substituent at 1344 cm⁻¹ and 1531 cm⁻¹, aromatic ring at 1604 cm⁻¹ and the aldehyde at 1703 cm⁻¹. The spectrum of benzene sulfonylhydrazide shows a characteristic N—H stretch at 3386 cm⁻¹, and sulfur-hydrogen stretches at 1307 cm⁻¹ and 1157 cm⁻¹. The spectrum of SH-3 reveals the carbonyl has been replaced by an imide stretch at 1634 cm⁻¹, while the characteristic stretches of both nitro (1509 cm⁻¹, 1345 cm⁻¹) and sulfonyl (1322 cm⁻¹, 1157 cm⁻¹) groups are present.

To form the tetrazole from the sulfonylhydrazone, a diazonium salt must first be synthesized (Scheme 5), following the reaction scheme developed by Ito et al. (S. Ito, Y. Tanaka, A. Kakehi, and K. Kondo, “A Facile Synthesis of 2,5-Disubstituted Tetrazoles by the Reaction of Phenylsulfonylhydrazones with Arenediazonium Salts,” BCSJ, vol. 49, no. 7, pp. 1920-1923, July 1976, doi: 10.1246/bcsj.49.1920., which is incorporated herein in its entirety by reference for all purposes). A word of caution, the diazonium salts used in this manuscript have chloride counterions for ease of purification, however these diazonium salts are unstable. If the synthesis solution of the diazonium salt is allowed to dry, the diazonium solids may precipitate and form a contact sensitive explosive. This reaction should not be scaled up beyond the milligram regime as this risks explosions significant enough to cause serious damage and injury. Safer alternatives in the form of tosylate, triflate or tetrafluoroborate counterions would be preferable for large scale synthesis as these compounds form more stable salts and consequently dramatically reduce the sensitivity of the diazoniums. The latter two counter ions are stable enough to fully dry and store the diazonium rather than synthesizing it prior to every reaction. These will complicate purification of the DATs but will be vastly safer to handle at scale.

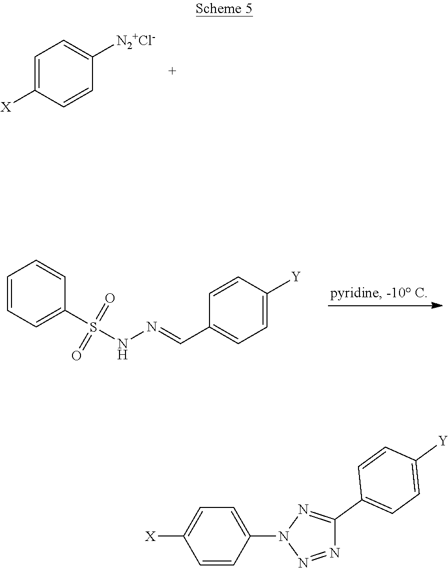

Diazonium salts were synthesized by dissolving the aniline in a water:ethanol:hydrochloric acid ternary solvent and chilling the solution to just above freezing. The exception to this rule is sulfanilic acid, which is entirely insoluble in most organic solvents and so was dissolved in a water-HCl solution. Even here it is barely soluble, so lower sulfanilic acid concentrations must be used and it must be sonicated for a significant length of time to fully dissolve. Once cool, sodium nitrite was added to form nitrous acid and thus create the diazonium salt. For anilines with electron-withdrawing substituents like sulfonyl or carbonyl groups, the solution was allowed to sit for several minutes to go to completion. The electron-withdrawing nature of the other substituent makes self-reactivity very slow and so more complete conversion of the aniline is possible. For other aniline derivatives (including aniline itself), this self-reactivity is significant and the diazonium salt must be used as soon as possible, preferably within seconds of adding the sodium nitrite.

The sulfonylhydrazone was prepared for the cyclization reaction by dissolution in pyridine. This solvent also acts a non-nucleophilic base that enables the cyclization reaction. Nitrosulfonylhydrozones showed substantial solvochromicity and turned a brilliant orange color upon dissolution in the pyridine, the rest yielded a light-yellow solution. This mixture was chilled to −10 C in an ice/brine bath and was stirred vigorously as the diazonium was added. Stable diazonium salts were added dropwise to minimize solvation heating and the effect of the acidic diazonium solvent. Upon addition of the diazonium salt solution, the solution briefly would turn deep red but would quickly revert to the yellow or orange color it typically had. I believe this to a pH effect as the color was reversible and quickly dissipated as the basic solvent neutralized the acidic diazonium solution. Unstable diazonium salts could not be added dropwise, lest the added delay cause all the diazonium to be consumed by self-reactivity. These diazonium salts were added as a single bolus to the pyridine solution, which instantly became a deep and opaque red. This color was likely due to the formation of azo-dye side products and was likely the primary contributor to the poor yields of these products. Both reactions were allowed to continue on ice for another 30 minutes, then were moved to a room-temperature stirrer and allowed to stir overnight.

Achieving a DAT with a benzyl amine functional group could not be achieved directly through the above method as it would also form a diazonium salt and react with other sulfonylhydrazones, forming an undesirable side product. To avoid this, the synthesis was performed with a nitroaniline derivative, which also has the benefit of having minimal self-reactivity as a diazonium salt. Once the tetrazole had been formed, the nitro group could be reduced to amines with a number of different reactions including an iron catalyst, by using sodium sulfide, or with palladium on carbon (Scheme 6). The iron-catalyzed method requires less dangerous reagents but is also harsher and risks degrading the tetrazole. Conversely the sodium sulfide method could be more selective in reduction but had the propensity to produce toxic hydrogen sulfide gas. The palladium on carbon required the use of both toxic hydrazine and pyrophoric palladium on carbon while being the most aggressive reaction, and thus was not considered. The two former reactions were both attempted and found to have comparable yields. With the fear of degrading the tetrazole assuaged, the iron catalyzed method was used as it was substantially safer.

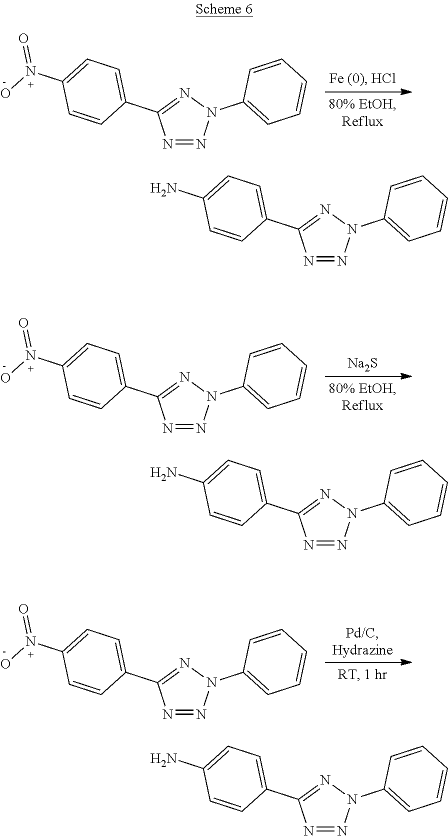

Prior to purification of the compounds, a reactivity test was generally performed to confirm that at least some 2,5-diaryltetrazole was created. On a small piece of Whatman filter paper, two small aliquots (˜0.5 μL) of the reaction solution were blotted. Next a small aliquot of polyethylene glycol diacrylate (PEG-DA) was blotted onto one of the DATs blots, while a second was blotted on a clean area of the paper to create three blots, one each of pure PEG-DA and reaction solution, and one where those were mixed. The filter paper was then viewed under 365 nm UV light, where no fluorescence should be observed in any blot. The filter paper was then exposed to shortwave UV light (302 or 254 nm) for 30 seconds to perform photoclick conjugation to the PEG-DA, then viewed again under longwave. The reaction was considered successful if only the blot containing the mixed compounds began to fluoresce. If the reaction solution alone began to fluoresce, it was considered contaminated, then discarded and resynthesized. Here a notable difference in fluorescence was observed. DATs that were derived from nitrosulfonyl hydrazones would fluoresce an intense red/orange, those derived from aminophenol-based diazoniums would fluoresce a bright canary yellow, while all other compounds would generally produce a blue/green fluorescence. LC-MS of the crude products was also used to confirm the synthesis was successful (LC-MS spectra can be provided to an examiner upon request and the spectra are described in the following paragraphs). Crude products were loaded into the liquid chromatograph as a general guide for the amount of impurities and their relative polarity compared to the desired product. The product was identified by the predicted mass and was ionized for MS by electrospray ionization (ESI). The ionization voltage of the ESI nozzle was low and as such whole mass ions as well as salt adducts were regularly seen with minimal fragmentation. Liquid chromatographs and mass spectra for all SH and DAT compounds can be found in Appendix 1.2 of Ostrovsky-Snider.

Once it was confirmed that the reaction produced DAT, purification was performed. The nitro derivatives would often precipitate in the pyridine solution and so would be removed by vacuum filtration and washed with a small amount of ethyl acetate. The other derivatives would generally stay in solution and so would be purified by extracting the solution with ethyl acetate and water. The organic layer was then washed with saturated brine to remove the residual acid, pyridinium and water. This solution was fully dried over sodium sulfate then dried under vacuum. This was often insufficient to achieve a pure compound as azo-dye side products would commigrate with the product, so flash chromatography with a 2:1 hexanes:ethyl acetate system would be used to purify the product. Certain products, particularly the carboxyl derivatives proved to be amenable to recrystallization, so this was used instead of chromatography. Sulfonyl derivatives had to be treated separately, they could not be extracted with ethyl/acetate and water as products, residual reactants and side products all favored the aqueous layer. Worse still, they could not be separated by normal phase chromatography as they would bind irreversibly to the silica gel stationary phase. Instead, the crude product was precipitated by the addition of acetic acid, dried under vacuum, then recrystallized from hot water. Crystals were of poor quality visually but showed high purity under LC analysis (images and data can be found in Ostrovsky-Snider and can be provided to an examiner upon request).

Yields for the DATs were generally good, with most losses likely due to purification rather than reaction inefficiency (Table 1). DAT-1 and 3 had very good yields due to their ease of purification. The poor yield on DAT-4 is very likely due to azo-dye side reactions taking place during the tetrazole formation. This was evident during purification as all other crude products were solids, while crude DAT-4 was a viscous oil due to the impurities dramatically reducing the melting point of the crude material. Somewhat lackluster yields were also obtained for DAT-6 and -7, as the spontaneous precipitation of the nitro-derivatives likely was incomplete, with a substantial amount of product left in the reaction solution. While more could likely be obtained via active precipitation, it was decided against in order to simplify the process. Likewise the yields of DAT-8 and -9 suffered as they relied on DAT-6 and -7 respectively as reagents.

TABLE 1
Yields of DAT synthesis reactions

| | DAT- | DAT- | DAT- | DAT- | DAT- | DAT- | DAT- | DAT- | DAT- | DAT- |
|---|---|---|---|---|---|---|---|---|---|---|
| Derivative | 1 | 2 | 3 | 4 | 5 | 6 | 7 | 8 | 9 | 10 |
| Yield | 94.1% | 69.3% | 92.5% | 40.2% | 63.7% | 67.6% | 58.6% | 61.3% | 50.3% | 74.2% |

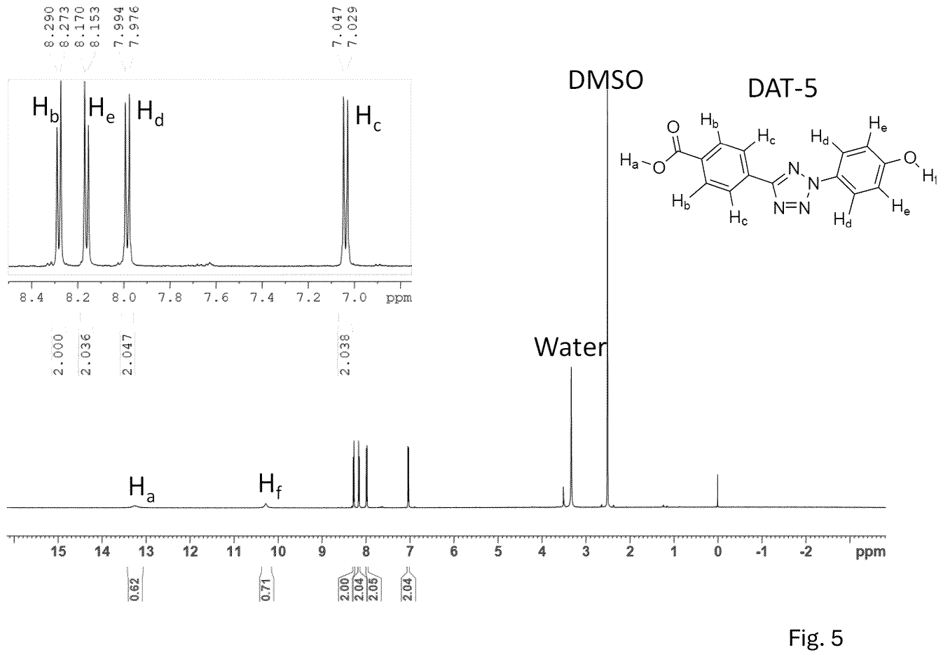

The purified compounds were analyzed by both FT-IR and NMR to confirm their identity and attempt to find characteristic peaks. The highly aromatic DATs had almost all their peaks in the aromatic region, and their high degree of symmetry made peak assignment difficult (FIG. 5). Assignments were made with splitting pattern and integrations, and when that left ambiguity J-coupling was used to distinguish the remaining signals. All compounds were analyzed by 1H-NMR and the spectra and proton assignments are provided in Appendix 1.3 of Ostrovsky-Snider.

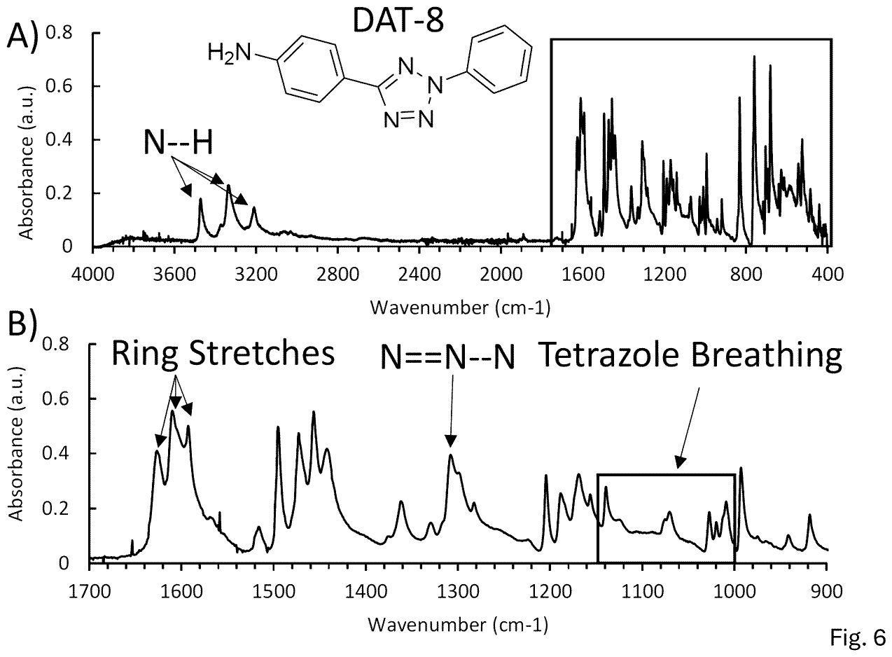

FT-IR was also equally difficult, as the high degree of molecular symmetry made the signals of many dipoles limited (FIG. 6A). The ring stretches of both aryl rings and the tetrazole near 1600 cm⁻¹ were often overlapping but could occasionally be separated into 3 distinct peaks (FIG. 6B). Similarly, the stretching of internal N—N══N structures could often be seen near 1300 cm⁻¹ but were often overlapped with other peaks. Finally, the three expected tetrazole breathing peaks from 1000-1150 cm⁻¹ were not distinguishable from other stretches in this region. FT-IR spectra of all compounds are shown in Appendix 1.1 of Ostrovsky-Snider.

### UV-Vis Absorbance

When these DATs were mixed with an alkene and illuminated with 302 nm light they quickly produced a bright fluorescent pyrazoline compound (Scheme 3). Polyethylene glycol diacrylate (PEG-DA) was chosen as alkene bearing substrates. While final devices would be primarily fabricated from Silk Methacrylate (SilMA), it was not well suited to many of the characterization techniques due to its insolubility in most organic solvents and aqueous solvents below pH 4.0, thus limiting the range of experiments that can be performed with it. It is also possible to make hybrid devices with both SF and PEG-DA as an alternative to methacrylating the SF, so investigation of the PEG-DA/DAT reaction and products is doubly warranted.

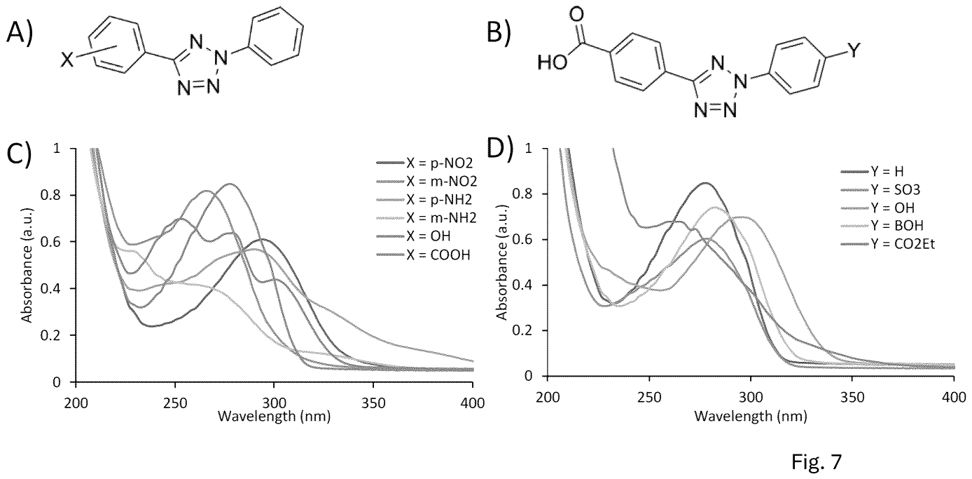

The effects of substitution on the spectral characteristics of the DAT derivatives and the pyrazoline adducts that result from the photoclick reaction of the DATs were investigated by UV-Vis and fluorescence spectroscopy. All derivatives showed an absorption peak between 250 and 300 nm, which corresponded to the activation wavelength of the photoclick process (FIG. 7). The absorption changes of the DAT precursors were tied to the electron affinity of the phenyl substituent on the ring attached to the 2-position of the tetrazole. Electron withdrawing substituents such as carboxyl or sulfate groups had similar or slightly hypsochromic absorption compared to hydrogen substituted DATs. Electron donation groups like hydroxyls had the opposite effect and the absorption peak displayed a bathochromic shift.

Substitution on the 5-phenyl ring also affected the absorption band of the photoclick process but did not correlate with the electronegativity of the substituents (Table 2). Substitutions on the para position, both electron donating and withdrawing tended to produce a bathochromic shift in absorption. Similarly, on the meta position electron withdrawing and donating substituents both produced a hypsochromic shift. This seems to indicate that resonance communication between the tetrazole and the functional group may be the deciding factor, and that both resonance stabilization of cationic and anionic states both lower the activation energy, and the lack of this resonance stability leaves only weak inductive effects that do not significantly impact the overall stability. There is also an unexpected triple absorption peak from DAT-10 (X═OH) unlike all other derivatives. It is not alone is being able to both accept and donate hydrogen bonds, or having ionizable groups that may be affected by pH, so the reason behind this behavior is unknown.

TABLE 2
Functional Group

| Derivative | DAT-1 | DAT-2 | DAT-3 | DAT-4 | DAT-5 | DAT-6 | DAT-7 | DAT-8 | DAT-9 | DAT-10 |
|---|---|---|---|---|---|---|---|---|---|---|
| X | -p-CO2H | -p-CO2H | -p-CO2H | -p-CO2H | -p-CO2H | -p-NO2 | -m-NO2 | -p-NH2 | -m-NH2 | -p-OH |
| Y | —H | —CO2H | —SO3— | —O— | —B(OH)2 | —H | —H | —H | —H | —H |

Excitation λmax (nm)

| | | | | | | | | | | |
|---|---|---|---|---|---|---|---|---|---|---|
| 1 | 280 | 265 | 279 | 296 | 285 | 296 | 266 | 294 | 271 | 253 |
| 2 | — | — | — | — | — | — | — | — | — | 281 |
| 3 | — | — | — | — | — | — | — | — | — | 300 |

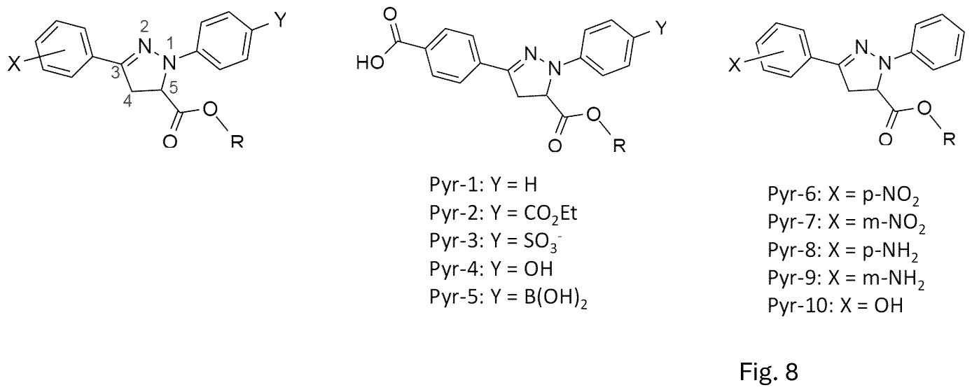

Upon addition of PEG-DA and irradiation with 302 nm light, the tetrazole becomes a fluorescent pyrazoline adduct. The loss of the nitrogens changes the numbering scheme of the pyrazoline. What had once been the 2-position in the DAT is now the 1-position, while the 5-position of the DATs is the 3-position in the pyrazolines (FIG. 8). Pyrazolines will be used throughout this remainder of this chapter to refer to PEG-DA bound pyrazolines but will be used in future chapters to refer to both PEG-DA and SilMA bound fluorophores.

### Kinetics of Formation

The rate of formation of the pyrazoline compounds was affected by the substituents on the 2- and 5-phenyl rings. DATs 1-10 were dissolved at 0.1 mg/mL in IPA and 10 mg/ml of PEG-DA was added, such that there would always be excess alkene and completion would require the total depletion of DAT. The reactions tend to follow a logarithmic increase in emission intensity until reaching reaction completion, where they then enter a stable plateau regime. We see a general trend where all DATs with a carboxyl substitution on the 5-phenyl position had much faster and reactions than those with any other substitution, with the exception of DAT-4 that had a hydroxyl on the 2-phenyl position. Interestingly we see that DAT-10 with a hydroxyl on the 5-phenyl position was the most rapid of the non-carboxylated derivatives. No clear trend exists between kinetics of reaction and electronegativity of substituents on either ring, or of UV-Vis absorbance values at 302 nm. The literature suggests there should be a trend based on electronegativity of the substituent on the 2-position of the DAT, however that is not observed here.

Certain DAT derivatives had absorption peaks closer to the 254-emission line of mercury lamps, so a comparison was made between excitation with the 302 and 254 nm bands. Reactions were slower across all derivatives, regardless of relative absorption values between 254 and 300 nm. This higher energy light might be suffering from additional scattering or the activation of other reactions, making it less efficient at initiating the click reaction. All spectra are located in Ostrovsky-Snider and can be provided to an examiner upon request.

### Fluorescent Pyrazoline

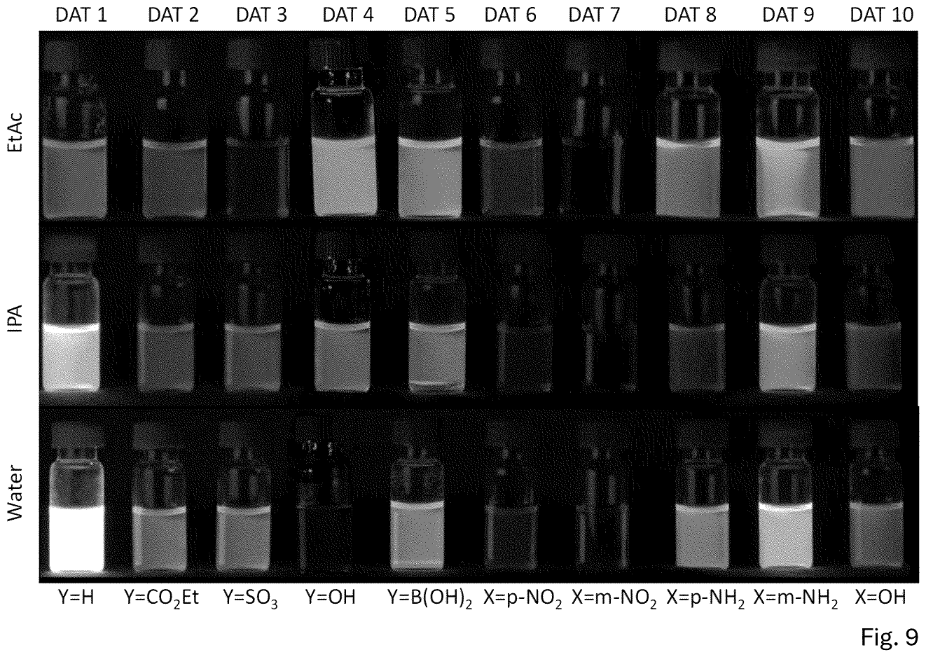

The pyrazolines created from the photoclick reaction showed a great variety of colors, from rich blues to orangish-reds. Once the PEG-DA fluorophores were diluted into various solvents, the intensity and emission wavelengths were both seen to shift (FIG. 9). A summary of the excitation and emission wavelengths of the various pyrazoline derivatives (Pyrs) is provided in Table 3. Solvent quenching and solvation related chromic shifts were also noted for the most of the pyrazoline derivatives. As is generally the case for red/orange fluorophores, the red/orange Pyr 6 was entirely quenched by solvents with hydrogen-oxygen bonds, and thus was only visible if dissolved in ethyl acetate. This is due to the vibrational energy of the oxygen-hydrogen bond being near to the excitation energy of the fluorophore and thus the excited state decays due to non-radiative energy transfer instead of emission. The other derivatives were generally not fully quenched but have a subdued emission intensity from only partial energy loss.

TABLE 3
Functional Group

| Derivative | Pyr-1 | Pyr-2 | Pyr-3 | Pyr-4 | Pyr-5 | Pyr-6 | Pyr-7 | Pyr-8 | Pyr-9 | Pyr-10 |
|---|---|---|---|---|---|---|---|---|---|---|
| X | -p-CO2H | -p-CO2H | -p-CO2H | -p-CO2H | -p-CO2H | -p-NO2 | -m-NO2 | -p-NH2 | -m-NH2 | -p-OH |
| Y | —H | —CO2H | —SO3— | —O— | —B(OH)2 | —H | —H | —H | —H | —H |

| | | | | | | | | | | |
|---|---|---|---|---|---|---|---|---|---|---|
| pH | Pyr-1 | Pyr-2 | Pyr-3 | Pyr-4 | Pyr-5 | Pyr-6 | Pyr-7 | Pyr-8 | Pyr-9 | Pyr-10 |

Excitation λmax

| | | | | | | | | | | |
|---|---|---|---|---|---|---|---|---|---|---|
| 3 | 378 | 378 | 379 | 327 | 365 | 430 | 354 | 432 | 354 | — |
| 10 | 372 | 345 | 373 | 367 | 368 | 456 | 368 | — | 375 | 342 |

Emission λmax in EtAc (nm)

| | | | | | | | | | | |
|---|---|---|---|---|---|---|---|---|---|---|
| — | 501 | 490 | 504 | 542 | 501 | 593 | — | 440 | 490 | 441 |

Emission λmax in IPA (nm)

| | | | | | | | | | | |
|---|---|---|---|---|---|---|---|---|---|---|
| 3 | 496 | 473 | 494 | 531 | 485 | 455 | — | 501 | 441 | 427 |
| 10 | 477 | 442 | 474 | 528 | 502 | 455 | — | 496 | 444 | 429 |

Emission λmax in water (nm)

| | | | | | | | | | | |
|---|---|---|---|---|---|---|---|---|---|---|
| 3 | 546 | 510 | 519 | 501 | 514 | — | 540 | 509 | 505 | 501 |
| 10 | 511 | 496 | 499 | 501 | 505 | — | 540 | 501 | 502 | 498 |

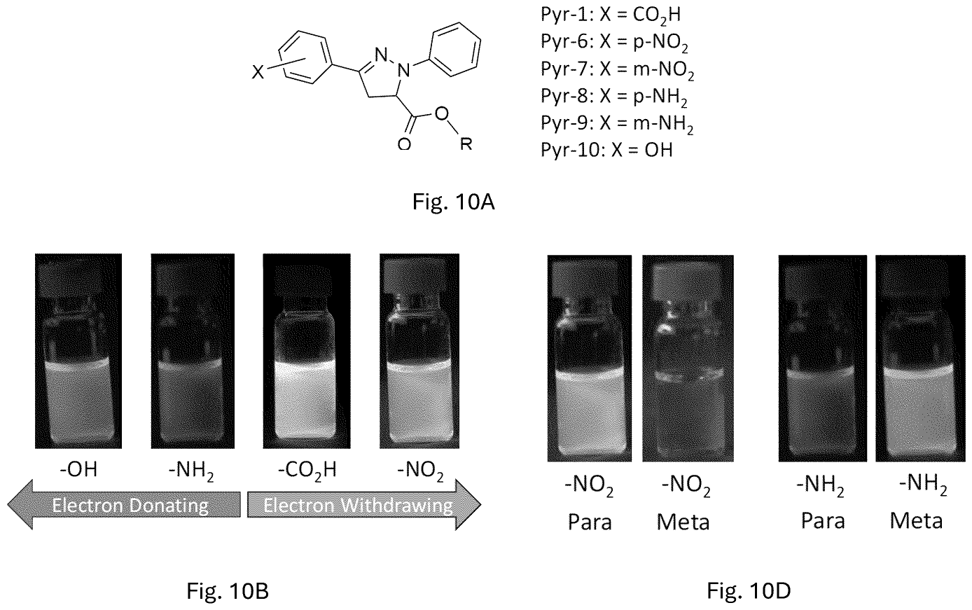

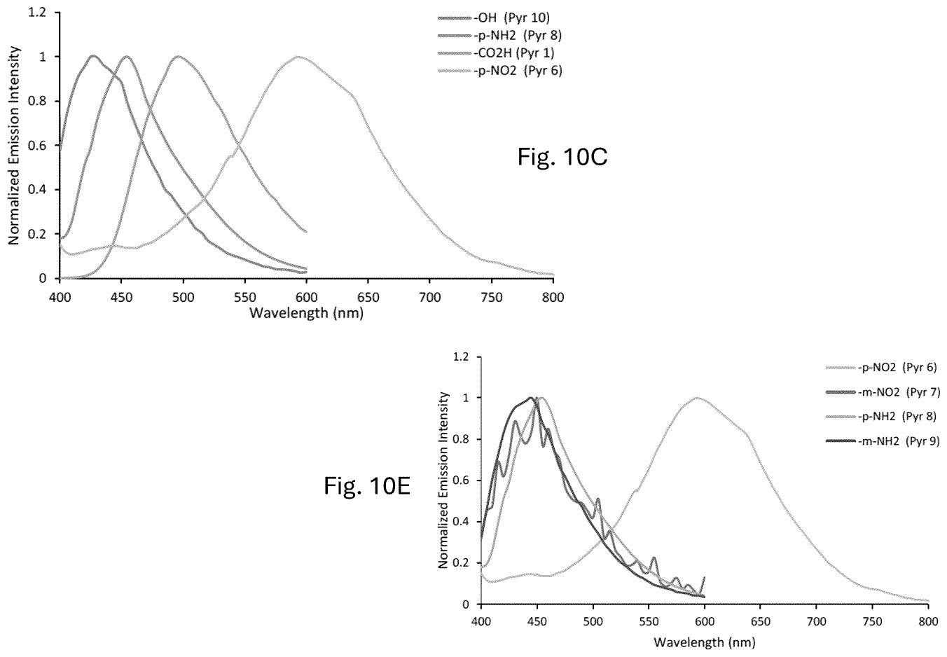

The emission wavelength of the fluorophores was also subject to a variety of factors, namely fluorophore structure, solvent quenching, and environmental pH. The structural differences in the composition are well correlated to electronegativity on both the 1-phenyl and 3-phenyl rings (FIG. 10). Mildly electron withdrawing substituents produced greenish fluorophores and strongly withdrawing substituents produced yellow/orange emission, while electron donating groups produced a blue green emission. Interestingly, the effect was consistent when the substituent was placed at either the meta or para positions on the 3 phenyl ring. This effect thus seems to be the result of direct electron density alteration of the pyrazole core from the altered distribution on the substituent phenyl rings.

Substitution on the 1-phenyl position displays a similar correlation between the degree of electron withdraw or donation by the substituent and the resulting wavelength of emission (FIG. 11). Electron withdrawing substituents on the para position produce a bathochromic shift, while electron donating substituents do the opposite. The primary exception to this is the sulfate substituted DAT-3 that is exceptionally electron withdrawing yet produces a blue-green fluorophore when the expected color would be a short-wavelength blue. The exact cause of this discrepancy is unknown.

Substituents on the 1-phenyl group show the reverse trend as those on the 3-phenyl group. Here electron donation produces the bathochromic shift. This is in line with previously described 1,3-diarylpyrazolines, where substitution on the 1-phenyl position alone was sufficient to shift the fluorescent emission peak across the entire visible spectrum. No sulfates or borates were investigated by the previous study however, so these may have additional resonance or hydrogen bonding phenomena that act counter to the overall trend. The ability to control the spectral emission from both phenyl rings shows that the pyrazoline is a highly flexible fluorophore that can be adapted for specific uses.

### pH Dependence

Finally, the pyrazolines derivatives display pH dependent quenching and chromic shifts, with the same behavior at play in both aqueous and organic environments. The pyrazoline itself has an ionizable site on the 2-N position on the tetrazole ring (FIG. 8), as well as the ionizable moieties like carboxylic acids in derivatives 1-5, primary amines in derivates 8 and 9, and alcohols in derivatives 4 and 10. While it is theoretically possible to protonate a nitro substituent, this requires concentrated mineral acids to achieve and therefore plays no role at these intermediate pHs. Each fluorophore has a conjugate acid/base pair, with each displaying a distinct fluorescence behavior, but solutions with both fluorophores present should display intermediate behavior characteristic of both. Based on Equation 1, the pKa of the compounds will correspond to the point at which we see a 50% transition between acid and base states.

$$pH = p {K}_{a} + {log}_{10} ( \frac{[ {A}^{-} ]}{[ HA ]} ) if [ {A}^{-} ] = [ HA ] , then pH = pKa \qquad ( 1 )$$

The expected pKa for these groups can be estimated by examining the pKa of a closely related compound. Phenol, benzoic acid and anilinium (the conjugate acid to aniline) have pKas of 10.0, 4.20 and 4.63 respectively, however are likely to be high estimates give the presence of the tetrazole. The tetrazole itself is strongly electron withdrawing, and therefore has a stabilizing effect on the conjugate bases of aromatic acids. Nitrophenyl derivatives are very strongly withdrawing, and so can be used as lower bounds. The pKa for 4-nitrophenol, 4-nitrobenzoic acid, and 4-nitroanilinium are 7.15, 3.41 and 1.01 respectively. Therefore, we estimate that carboxyl derivatives will have a pKa in the range of 3.4-4.2, phenol derivatives will have a pKa in the range of 7.15-10.0 and aniline derivatives will have a pKa in the range of 1.01 to 4.63. Literature also suggests that the 2-N amine in the pyrazoline ring can also be protonated, with a reported pKa of similar compounds being below 2.

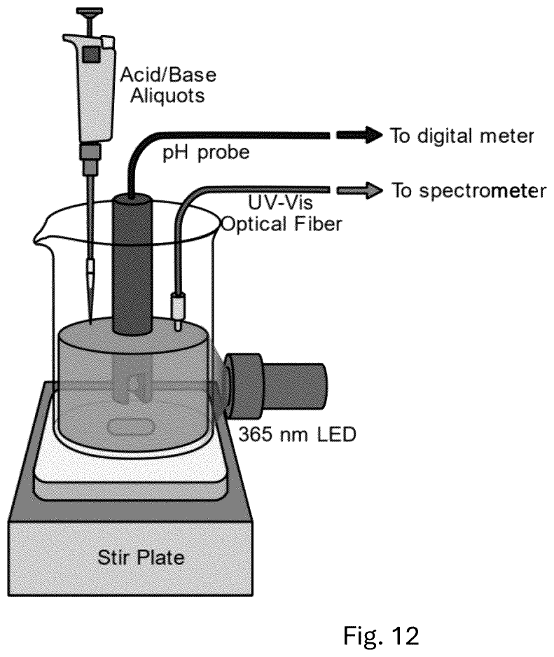

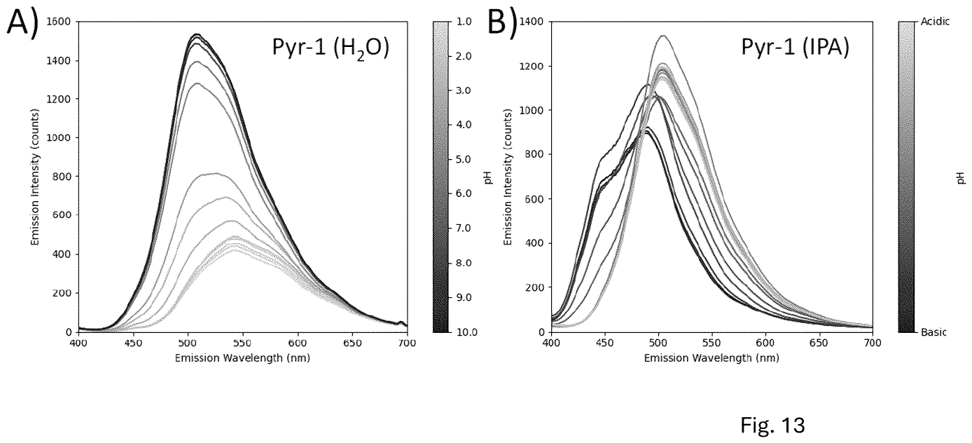

To determine the exact pH dependance of the fluorescence transition, fluorescence was monitored during an acid-base titration with a custom optical set-up (FIG. 12). This was done in both IPA and DI water, the latter of which enabled direct pH measurement alongside the fluorimetry. To perform the titration, PEG-DA-bound-pyrazoline was added to the solvent at a final concentration of 25 μM in 50 mL of solvent. A UV-Vis spectrometer was attached to a quartz optical fiber and the end was immersed in the solution. A 365 nm lamp illuminated the solution and was oriented at 90 degrees to the optical fiber to minimize spurious exposure from the excitation light. If the solvent was DI water then an electrochemical pH probe was also inserted into the beaker. The pH of the solution was changed with the addition of 5 μL aliquots of 1 M para-toluenesulfonic acid or potassium hydroxide dissolved in the same solvent as the analyte. Concentrated acid and base solutions were used to minimize the effect of dilution on the emission signal. After addition of the pyrazoline derivative, base aliquots were added until the emission signal stayed constant to 3 subsequent additions. Then acid aliquots were added until 3 subsequent additions of acid did not produce a chance in emissions. Typical titrations would require around 80 μL of acid to be added once the basic steady state was reached, resulting in a total dilution of about 0.2%, which was considered negligible for subsequent calculations. Output of the pyrazoline titrations showed two distinct fluorescent peaks that shifted relative intensity at the pH was changed (FIG. 13). Titration spectra for all derivatives can be found in appendix A1.4 of Ostrovsky-Snider.

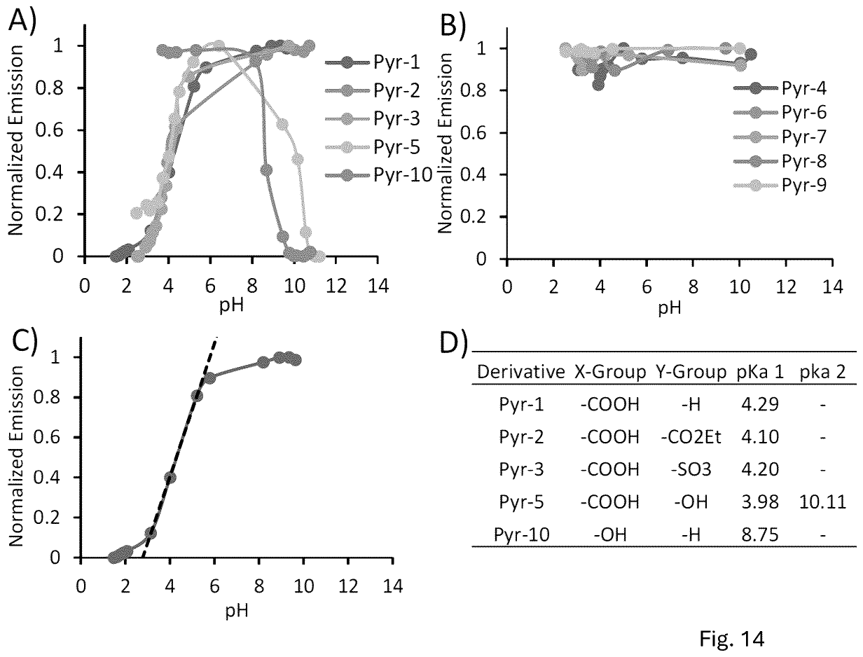

Resulting titrations generally produced either a sigmoidal curve with high and low pH stable regimes (FIG. 14A) or a static, pH independent fluorescence (FIG. 14B). The linear portion of the titration curve was fitted to a line of best fit (FIG. 14C) and the pKa was determined by normalizing the emission at high intensity to 1 and low intensity to 0, then calculating the pH at which emission would equal 0.5. The pKa values estimated by fluorescence are generally within their estimated pKa windows, with the exception of the amino derivatives (FIG. 14D). No evidence of an acidic form was noted from the titration experiments, despite reaching pHs as low as 2.5. This stands contrary to the evidence from additions of acid to DAT-8-derived-pyrazolines in IPA, which do display acid/base sensitivity and a distinct chromatic shift. The likely explanation for this is that the higher pKa of IPA compared to water stabilized the conjugate acid in IPA solutions and insufficient acid was added to the water solution to reach the edges of the buffer window. The other amine, DAT-9-derived-pyrazoline, shows no acid/base behavior in either water or isopropanol and is therefore more likely to be lacking any acid-base chromatic shifts.

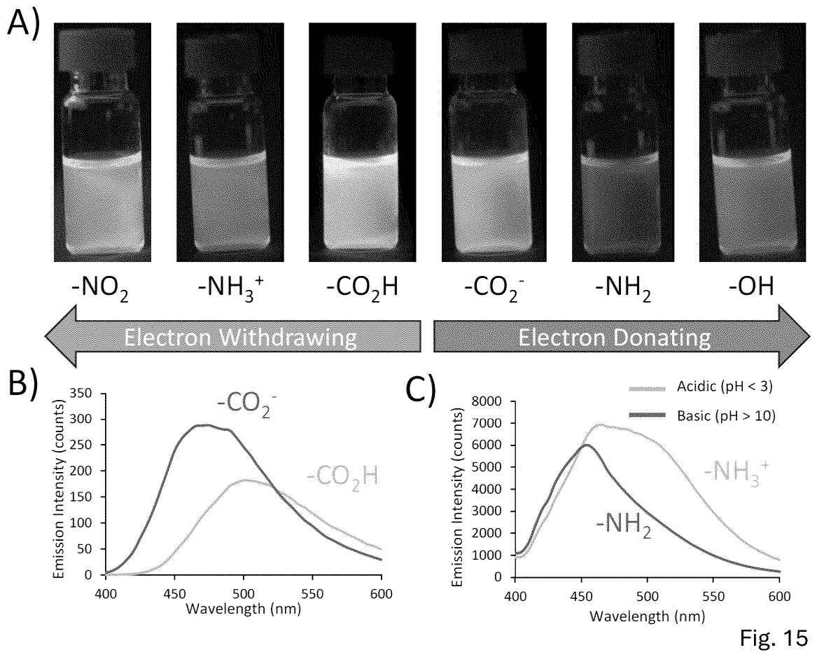

Finally, the borate functionalized pyrazoline DAT-5, displayed a 3 distinct regions of emission, with a highly emissive state centered at pH 6 and both acidic and basic states of low-emissivity. The high-pH regime shows a pKa of 10.11 while the low pH regime had a pKa of 3.98. The low pH regime was clearly due to the carboxylate group, as it showed a nearly identical pKa to the other carboxylated derivatives. The high pH regime is therefore due to the borate group picking up an additional hydroxyl, and very nearly matches the pKa of boric acid (9.15). The lack of a pKa for Pyr-4 is due to the almost complete quenching of that fluorophore in water, whereas in IPA it displays clear pH dependent behavior. Incorporating this data into that already seen for different functional groups, shows the pH-based chromic shifts match the larger trend and electronegativity can be used to predict changes in emission frequency (FIG. 15).

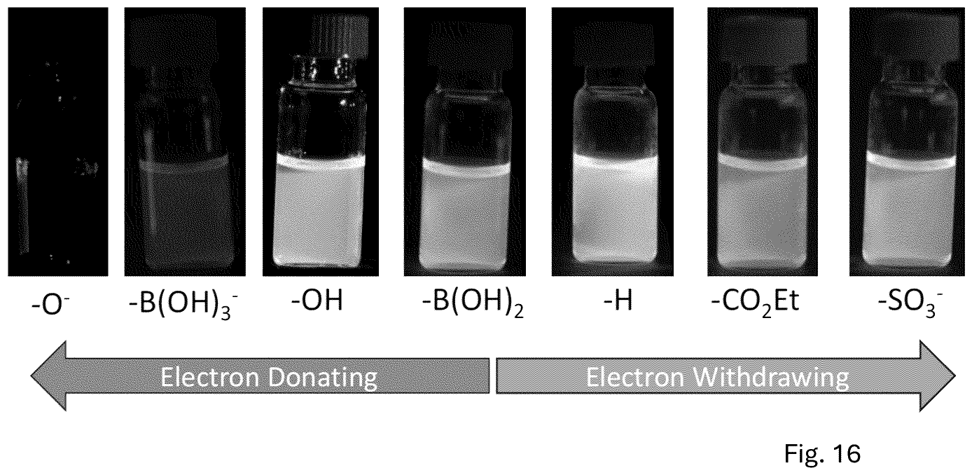

Changes to the emission of from pH effects on the 1-phenyl position are less clear but I would argue reinforce the same point (FIG. 16). The ionization of Pyrs 4 and 5 both increase the electron donation of their functional groups. As these derivatives were already emitting in the yellow and green regions respectively, if they followed the same trend, we would expect the derivatives to become orange/red. However, as we have seen before orange/red derivatives are strongly quenched by solvents with hydroxyl functional groups. Thus the quenching of fluorescence of these groups is also supportive of the hypothesis.

### Conclusions

Various derivatives of the DAT molecule were synthesized and shown to form fluorescent pyrazoline-bearing materials upon a photo mediated click reaction with acrylate bearing polymers such as SilMA and PEG-DA. The various DAT derivatives were shown to have variable reaction rates that were not clearly correlated with the spectral properties of the DAT or the resulting Pyr. The fluorescent pyrazolines showed solvent sensitive fluorescence, displaying a general bathochromic shift when introduce to more polar solvents and quenching of orange/red emission when introduced to solvents with hydroxyl functional groups.

The substitutions were shown to modulate the fluorescent behavior by altering the electron density within the pi systems of the pyrazoline. Electron withdrawing substituents on the 3-phenyl position of pyrazolines produced a bathochromic shift, while the those on the 1-phenyl position produced a hypsochromic shift.

The ionizable groups on the pyrazolines enabled them to have pH-dependent fluorescence, which followed the general trend seen with derivatives before. If the ionizable group was on the 3-phenyl position and became more electron withdrawing it would produce a bathochromic emission shift, with the 1-phenyl derivatives doing the opposite.

### Future Directions

The initial aim of this work was not to create a fluorescent material, that was a mere side-effect of the photoclick reaction, however that aspect of the chemistry came to dominate the work as a whole. As a result, the fluorophores produced here have a limited fluorescent range and some are completely quenched in biological media. Rational design of fluorophores could dramatically improve the performance of these compounds. This could most easily be done by the addition of large, sterically inhibiting groups onto the phenyl rings. These groups would restrict the access solvent and biomolecules have to the fluorescent core of the molecule and thus limit the possibility for resonant energy transfer, which is responsible for much of the environmental quenching in red/orange fluorophores, particularly when dissolved in solvents with hydroxyl functional groups. This would allow the engineered creation of a variety of red and potentially infra-red fluorescent molecules that could be photopatterned via the photoclick reaction.

### Functionalization of Silk Fibroin

### Chemical Modification of Silk Fibroin

Silk fibroin has remarkable innate properties, however many applications find it necessary to chemically modify the SF to perform unorthodox behaviors such as photopolymerization, promoting cell attachment, thermal gelation, conductive polymer doping and many more. While SF is a very large protein it has a very limited amino acid repertoire, with glycine, alanine, serine, and tyrosine composing 93.6% of the residues by number.

Of the 5263 amino acids present in the heavy chain of SF, only 12 of that number are lysine, which is among the most common targets of protein modification chemistries like maleimides, isothiocyanates, and carbodiimides. Despite this lack, it is still possible to crosslink fibroin with carbodiimide chemistry, or decorate it with various carboxylic acids like hyaluronic acid, heparin, and alginic acid (Scheme 7A). However, SF has over 30 glutamic acid and 25 aspartic acid residues, allowing activation by 1-ethyl-3-[3-(dimethylamino)propyl]carbodiimide (EDC) and N-Hydroxysuccinimide (NHS), and subsequent reaction with amines (Scheme 7B). The greater number of reaction sites allows for over 4-fold higher functionalization rates and has been used to attach reduced-glutathione, IKVAV peptides, and amine-terminated polyethylene glycol.

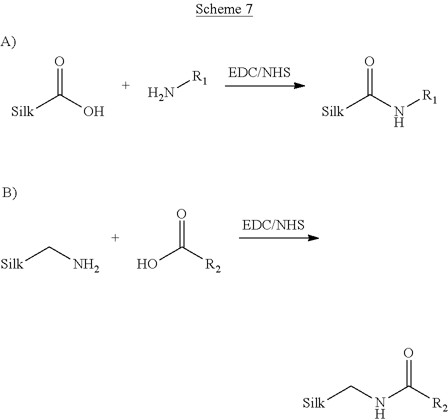

The free alcohol groups on the highly abundant serine and tyrosine residues (635 and 237 per heavy chain respectively) can also be functionalized with isocyanates (Scheme 8A), and anhydrides (Scheme 8B). While terminal amine groups are the stronger nucleophiles, alcohol groups are sufficiently nucleophilic to attack these highly electrophilic compounds to form urethane and ester bonds respectively. This has been used to successfully graft acrylate monomers onto SF with 2-(Methacryloyloxy)ethyl isocyanate [82], and gallic acid esters with gallic-acid anhydrides.

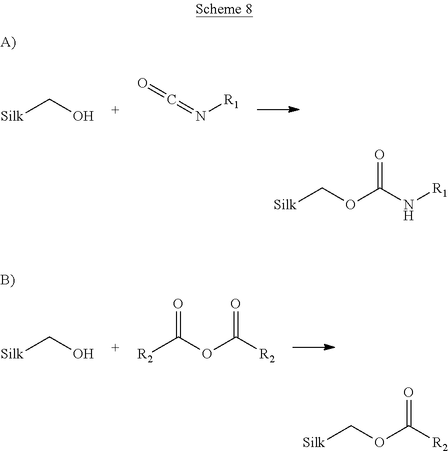

Finally the tyrosine group offers a readily accessible site for functionalization via aromatic substitution. Diazonium salts can readily form azo-dyes with tyrosines in silk (Scheme 9A) and has been used to introduce azides for CuAAC and SPAAC reactions, as well as forming azo-benzene inspired photoactuators. Beyond this, enzymes such as horse radish peroxidase, tyrosinase or laccase can form direct aryl bonds between tyrosine and aromatic chemicals like dopamine, phenol red and autologous crosslinking with other tyrosine residues (Scheme 9B). Lastly, diazonium elimination reactions catalyzed by TiCl₃ can allow direct arylation of tyrosines via the Gomberg Bachman mechanism (Scheme 9C).

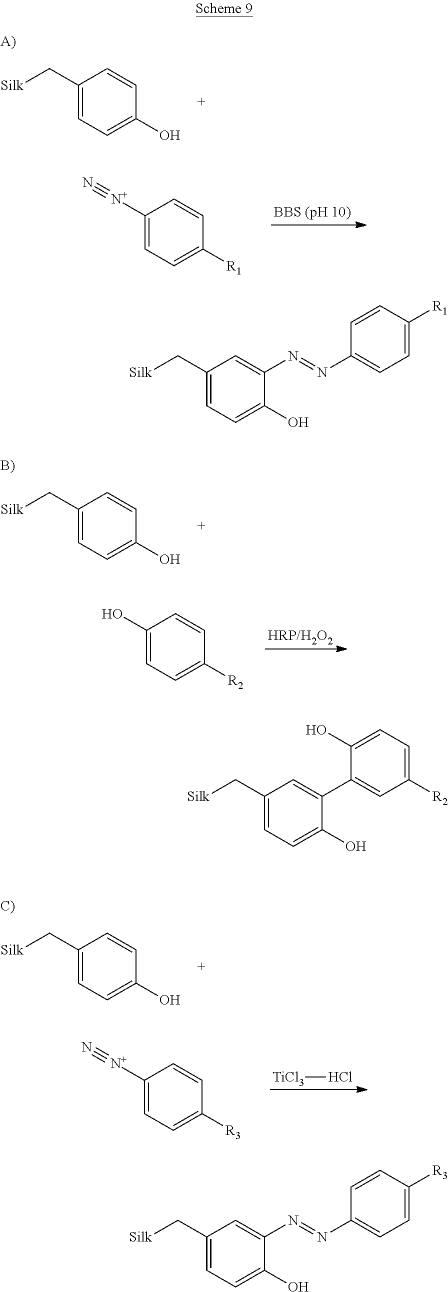

### Molecular Weight Analysis

Molecular weight is often crudely referred to as the size of molecule, however in polymer science the need to deal with large populations of heterogenous molecules requires us to work with population averages. Different techniques for measuring molecular weight will give a different population average. For example size exclusion chromatography will give the number average molecular weight (Mn), which is the sum of the products of each molecule's weight (Mi) by the number in solution (Ni), divided by the total number of molecules (Equation 2). Alternatively, light scattering techniques measure the mass average molecular weight, which is defined as the sum of the product of the number of molecules with the square of each molecules weight, divided by the sum of the product of the number of molecules of each weight with their weights Equation 3: Definition of weight average molecular weight. While all different techniques measure averages in different ways, all measurements converge when there is only a single molecular weight in the population. A population with only a single molecular weight is referred to as monodisperse, whereas a population with different molecular weights is referred to as polydisperse.

$${M}_{n} = \frac{\sum {N}_{i} {M}_{i}}{\sum {N}_{i}} \qquad ( 2 )$$

$${M}_{w} = \frac{\sum {N}_{i} {M}_{i}^{2}}{\sum {N}_{i} {M}_{i}} \qquad ( 3 )$$

Dispersity (Ð) is a measure of the variability in the molecular weights in a mixture. A common measure of dispersity is the ratio of the weight average molecular weight over the number average molecular weight (Equation 4: Definition of dispersity.). While all weight averages converge for a single population, as soon as the is diversity in the mass sizes the averages will diverge. The number average grows linearly, while the weight average grows geometrically, such that the weight average will always be larger than the number average. In monodisperse population this ratio will be 1, but as the variance in the population increases the weight average will diverge more from the number average and thus the dispersity will grow. Thus, the higher the dispersity, the more variability in molecular weight. The effects on polymers of increased dispersity are numerous, but tend to result in softer and weaker materials than a monodisperse polymers of the same weight would have. Polydispersity is common amongst artificial polymers, however natural polymers like proteins tend to be monodisperse due to their coded stepwise production by ribosomes translating a mRNA sequence. This monodisperse nature enables more simplistic methods of molecular weight analysis.

$$Ð = \frac{{M}_{w}}{{M}_{n}} \qquad ( 4 )$$

The most common molecular weight analysis technique for biomolecules like proteins is electrophoresis, specifically sodium dodecyl sulfate-polyacrylamide gel electrophoresis (SDS-PAGE). Here a polyacrylamide hydrogel is cast with sufficient crosslink density to form a network with pores of a similar size to denatured proteins. The proteins are denatured and charged by the detergent molecule sodium dodecyl sulfate (SDS), giving an approximately equal charge/mass ratio to all proteins and allowing them to separate by mass alone. To determine the absolute size of a protein, a set of standards (often called a ‘molecule weight ladder’, or just ‘ladder’) are applied to the gel to correlate migration distance with mass. Thus a protein's mass can be determined by comparing how far into the gel it migrated compared to known standards.

Silk fibroin (SF) being a protein, would seem highly amenable to this process, however the way in which it is processed complicates the analysis. Like all other proteins, SF is monodisperse when it is synthesized by the silk moth, however the process of degumming the SF degrades the backbone of the silk, causing variation in the size of SF molecules. While monodisperse proteins appear as narrow bands in an SDS-PAGE gel, SF will appear as a smear that runs the length of the gel. Ideally, measuring intensity variations across this smeared band could give you the, but variations in staining intensity make these values very difficult to extract. In practice you are left with an average molecular weight that is highly uncertain and difficult to measure.

Another option that is more commonly used to analyze synthetic polymers is size-exclusion chromatography (SEC). Like SDS-PAGE, the analytes are separated by size due to their size-based resistance to movement through the column matrix, however in SEC the size inhibition is reversed. The SEC column is a collection of gel particles that have a variety of pore sizes. Smaller molecules can enter many of the pores and become temporarily trapped and their elution is significantly delayed, while larger molecules cannot enter any but the biggest pores and their elution is minimally delayed. Thus, the molecules that flow through the column the fastest are the largest, while those that take the longest are the smallest.

Determining molecular weight from SEC can be done in several general ways with the most common being, 1) comparison to standards (standard calibration), or 2) independent molecular weight measurement (universal calibration). Standard calibration works by analyzing the propagation of a number of monodisperse standards of known mass and comparing elution times to these standards. This techniques is widely used due to it's simplicity, as it can be done without any specialized equipment beyond the ordinary detectors present on most HPLCs. It is however complicated by the fact that standards must be chosen carefully. Ideally SEC achieves separation purely by size, however intermolecular forces between the polymer analytes and the stationary gel will inevitably cause some simultaneous separation. Further, polymers do not always take the same shape in solution, some may adopt a soft-globule conformation that may be more likely to interact with pores, while others may be more akin to rigid spheres that avoid interaction. Thus it is crucial that the chemical nature (i.e. the polarity, surface functionality, etc.) and hydrodynamic nature (e.g. dissolved conformation, solvent-solute interactions, etc.) of the standards be as similar to the analytes as possible.

These difficulties may be resolved by universal calibration. To perform this, independent molecular weight analysis is performed on each solution component as it exits the column by a detector that measure multi-angle laser light scattering (MALLS). Particles that are much smaller than the wavelength of incident light scatter that light proportionally to their molecular weight, as described by the Rayleigh equation. With an absolute and independent method of measuring the molecular weight, standards are no longer needed and both molecular weight and dispersity can be derived from the data. While this technique is powerful, it requires an expensive and highly specialized detector that is uncommon outside of laboratories dedicated to polymer chemistry and molecular material science.

### Methods and Materials

All chemicals were purchased from Sigma Aldrich or Fischer Scientific. Bombyx mori silk cocoons were purchased from Tajima Shoji Co. (Yokohama, Japan).

### Isolation, Synthesis, and Purification

### Isolation of Silk Fibroin and Silk-Methacrylate (SilMA) Synthesis

To 4 L of deionized water, 8.5 g of sodium carbonate was added and stirred until dissolved, then the solution was brought to a vigorous boil. Bombyx mori cocoons were shredded into 1 cm² size pieces then boiled in the carbonate solution for 30 minutes unless otherwise specified. The fibers were removed from the boiling water and immediately placed into room temperature water to reduce the temperature as fast as possible. The fibers were rinsed 3 times in deionized water then allowed to dry overnight. The next day the fibers were immersed in sufficient 9.3 M lithium bromide solution to create a 20% w/v solution and placed in a 60 C oven to speed dissolution. If the silk was to be methacrylated, the solution was placed on a heated stir plate, a stir bar was added, and the solution heated to 60 C. Once heated the solution was stirred vigorously and glycidyl methacrylate was added to a final concentration of 540 mM. The solution was allowed to react for 3 hours then removed from the heat. The lithium bromide was then removed from both methacrylated and unmodified fibroin by placing the fibroin solution into a dialysis bag (3500 MWCO) and immersing in 4 L of DI water for 2 days, with the water changed a total of 6 times. The fibroin was centrifuged at 11,000 RCF for 20 minutes, then vacuum filtered through a nitrocellulose filter (5 μm pores), and stored at 4 C until use.

Carboxylation of Silk Fibroin with Chloroacetic Acid

Silk fibroin was carboxylated using a previously described protocol [93]. Briefly, 4.3 mL of sodium hydroxide (10 M) was added to 10 mL silk fibroin solution (5% w/v) under constant stirring. To this solution was added 13.51 g of chloroacetic acid and left on constant stirring at room temperature for 1 hour. The reaction was terminated by the addition of 100 mg of dibasic sodium phosphate, chilled on an ice bath, and the pH adjusted to 7.0 with concentrated hydrochloric acid. The solution was then placed into a dialysis bag (3500 MWCO) and immersed in 1 L of DI water for 2 days, with the water changed a total of 6 times. The fibroin was centrifuged at 11,000 RCF for 20 minutes, then vacuum filtered through a nitrocellulose filter (5 μm pores) and stored at 4 C until use.

Carboxylation of Silk Fibroin with Succinic Anhydride

Fibroin was carboxylated using a previously established protocol [94]. Briefly, 1 g of lyophilized fibroin was added to 6.25 g of molten (100° C.) anhydrous 1-butyl-3-methylimidazolium chloride (BMIM-Cl). When fully dissolved the solution was then diluted with 15 mL of anhydrous dimethylformamide (DMF). To this solution was added 21.4 mg of sodium dodecyl sulfate (SDS) and 2 g of succinic anhydride. The solution was stirred continuously on heat for 1 hour then allowed to cool to room temperature. The cool solution was diluted with 20 mL of deionized water then placed in a dialysis bag (3500 MWCO) and dialyzed against 1 L of DI water. Dialysis continued for 2 days, changing the water 5 times. The silk precipitated during dialysis, so it was dried at room temperature then dissolved in lithium bromide (9.3 M, 60° C.) to a final concentration of 20% w/v. The fibroin solution was again placed in a dialysis bag (3500 MWCO) and dialyzed against 1 L of deionized water for 2 days, changing the water 5 times. The resulting solution was removed from the dialysis bag, centrifuged at 11,000 RCF for 20 minutes, then vacuum filtered through a nitrocellulose filter (5 μm pores), and stored at 4 C until use.

### EDC/NHS Coupling SF to DAT-1

In a small conical vial 23.8 mg of DAT-1 dissolved in 100 μL of DMSO, then added to 1 mL of MES buffer (pH 5.6, 0.1 M). To this mixture was added, 19.4 mg of EDC and 14.3 mg of NHS, the vial wrapped with foil and mixed by gentle rocking for 1 hour. This solution was then added to 1.5 mL of SF (16.7 mg/mL) in PBS (pH 7.5, 0.17 M), the vial covered with foil and mixed by gentle rocking for 24 hours at room temperature. Excess DAT-1 that had precipitated in solution was pelleted by centrifugation at 1000 RPM for 30 seconds, then the supernatant was purified by desalting with a PD-10 column equilibrated with DI water.

### EDC/NHS Coupling SF to DAT-8

Silk fibroin solution was diluted with 2-(N-morpholino)ethanesulfonic acid (MES) buffer (pH 5.6) and DI water to a final concentration of 10 mg/mL SF and 0.1 M MES at a final volume of 2.5 mL. To this was added 19.4 mg EDC and 2.8 mg NHS, and the reaction solution was mixed by gentle rocking at room temperature for 15 minutes. The silk solution was purified by desalting in a PD-10 column pre-equilibrated with PBS (pH 7.5, 0.1 M). Separately 23.8 mg of DAT-8 was dissolved in 100 μL of DMSO, then added to the silk in PBS solution. Vial was then covered with foil and mixed by gentle rocking at room temperature for 24 hours. Excess DAT-8 that had precipitated in solution was pelleted by centrifugation at 1000 RPM for 30 seconds, then the supernatant was purified by desalting with a PD-10 column equilibrated with DI water.

### HBTU Coupling

Carboxylated silk fibroin was lyophilized, and 40 mg was dissolved in 2 mL of DMSO to create a 20 mg/mL solution. To this solution was added 7.5 mg of Hexafluorophosphate Benzotriazole Tetramethyl Uronium (HBTU) and 7.0 μL of N,N-diisopropylamine. The solution was mixed until solids dissolved and then allowed to react for 15 minutes at room temperature. To this solution was added 10 mg of DAT-8, the vial covered in foil and allowed to react under gentle mixing for 24 hours at room temperature. The solution was then diluted with 8 mL of DI water, and the precipitants were pelleted by centrifugation at 1000 RCF for 30 seconds. The supernatant was placed into a dialysis bag (3500 MWCO) and dialyzed against 1 L of DI water for 2 days, changing the water 5 times.

### Diazonium Coupling

Aqueous diazonium salts were prepared by dissolving DAT-8 or DAT-9 (59 mg, 0.25 mmol) in 1.5 mL of 50% ethanol solution. To this solution was added 250 μL of concentrated hydrochloric acid, then the solution was placed into an ice bath for 15 minutes. Simultaneously a silk fibroin solution (8 mL, 5% w/v) in BBS (0.1 M, pH 10) was placed on ice. Once cool 260 μL of 4 M sodium nitrite was added to the DAT solution and the solution was quickly mixed by inversion and replaced on the ice for 5 minutes to form the DAT-diazonium. The diazonium solution was added to the silk solution, quickly mixed by inversion then replaced on the ice. The solution rapidly became a reddish orange and allowed to react for 45 minutes, over which time it darkened to a deep burgundy color. The silk fibroin was then purified by dialysis against 1 L of DI water for 2 days, changing the water 5 times.

Alternatively the diazonium salt could be prepared as a triflate salt and stored dry prior to the azo dye reaction by following the process developed by Filimonov et al. [95]. To synthesize diazonium triflate, 237 mg (1 mmol) of DAT-8 or DAT-9 was dissolved in 10 mL of glacial acetic acid. This was cooled to 14 C by a solid-state cooler, then 107 μL (1.2 mmol) of tert-butyl nitrite and 306 μL (1.2 mmol) of triflic acid was added and stirred continuously. The reaction was monitored by TLC and terminated once all the reactants had been consumed, typically 30 minutes. The diazonium triflate salt was precipitated by pouring the acetic acid into 100 mL of diethyl ether. The precipitant was isolated by vacuum filtration and washed with a small volume of diethyl ether, yielding a light-brown solid. The remaining solvent was removed under vacuum and the diazonium salt was stored at room temperature in an opaque container until used.

To form an azo-dye with the triflate, 8 mL of silk fibroin (5% w/v) in BBS (0.1 M, pH 10.0) was chilled to 0 C in an ice bath. To the silk solution was added 96.5 mg (0.25 mmol) of DAT-diazonium triflate and mixed by inversion until all solids had dissolved. The solution immediately became reddish orange was returned to the ice and allowed to react for 45 minutes, over which time it darkened to a deep burgundy color. The silk fibroin was then purified by dialysis against 1 L of DI water for 2 days, changing the water 5 times.

### Gomberg-Bachmann Arylation

This protocol is a modified version of the protocol described by Wetzel et al. (A. Wetzel, G. Pratsch, R. Kolb, and M. R. Heinrich, “Radical Arylation of Phenols, Phenyl Ethers, and Furans,” Chemistry—A European Journal, vol. 16, no. 8, pp. 2547-2556, 2010, doi: 10.1002/chem.200902927, which is incorporated herein in its entirety by reference for all purposes). A two necked round-bottom flask was filled with 6 mL of aqueous solution containing either phenol (100 mM) or silk fibroin (50 mg/mL), a stir bar was added and the flask was sealed by placing rubber septa in both openings. Two needles were pierced through the septa, with one side containing a longer needle that was able to reach below the solution level. Argon was flowed through the longer needle for 10 minutes to sparge the solution and purge the atmosphere of oxygen. After sparging, 4 mL of TiCl3 (1 M in 3 M HCl solution) was added to the solution under constant stirring. Next a solution of DAT-diazonium triflate was made by the methods described above then 48 mg was dissolved in 5 mL of deionized water and added dropwise to the mixture. The reaction was allowed to proceed for 30 minutes then was terminated by the addition of 20 mL of stop solution (2.0 g sodium hydroxide, 2.0 g sodium sulfite), which precipitated the titanium. Phenol containing solutions were extracted with ethyl acetate then dried over sodium sulfate and solvent was removed under vacuum. Silk solutions were filtered to remove the titanium precipitate, then neutralized with citric acid and purified by desalting with a PD-10 desalting column equilibrated with DI water.

### Chromatography

### Size Exclusion Chromatography

Analysis was performed with an Agilent 1260 Infinity II high performance liquid chromatograph equipped with a variable wavelength detector and separated with an Agilent Aquagel Mixed-M size exclusion column (PL1149-6801). Mobile phase (8.0 M urea, 10 mM sodium phosphate, pH 7.4) was filtered through a 0.22 μm nitrocellulose filter prior to use. Samples and standards were diluted to 1 mg/mL in mobile phase and filtered through a 0.22 μm PVDF filter prior to analysis. 10 μL of sample was injected onto the column, which was maintained at 25 C, isocratically eluted at 1.00 mL/min for 14 minutes and detected at 280 nm unless stated otherwise.

### Liquid Chromatography Mass Spectrometry (LC-MS)

Crude compounds were analyzed by liquid chromatography-mass spectrometry (LC-MS) to confirm mass. Each sample was dissolved in a combination of mobile phase solvents, depending on solubility. This sample was then separated by an Agilent 1260 Infinity II, using a Poroshell 120 C18 column kept at 30±2° C. Formic acid (LC-MS grade, Sigma Aldrich) was added to a concentration of 0.1% to the mobile phase solvents acetonitrile (LC-MS grade, Sigma Aldrich) and water (LC-MS grade, Sigma Aldrich) to improve ionization. Mobile phase was flowed as a gradient from 5% acetonitrile in water to 100% acetonitrile over 14 minutes followed by isocratic elution of acetonitrile for a further 2.5 minutes at 1.00 mL/min. Elution was detected by an Agilent 1260 Infinity II diode array detector taking continuous spectra from 190-600 nm with a resolution of 2 nm. Mass spectrometry was done with an Agilent 6230B time-of-flight mass spectrometer, with an ionization mode suitable for each compound. All compounds were ionized by electrospray at 3.5 kV, fragmented at 30 V, with carrier gas at 325° C. flowing at 8 L/min and sheath gas at 350° C. flowing at 11 L/min.

### Results and Discussion

### Synthesis of Silk Fibroin Methacrylate

Silk fibroin methacrylate (SilMA) has been synthesized in a number of ways with goal of installing the useful methacrylate moiety onto silk fibroin and enabling it to cross-link via radical polymerization. A common method uses isocyanoethyl methacrylate to attach methacrylate to the various nucleophilic side chains, primarily the nucleophilic amines in lysine, arginine and the N-terminus. While effective, this method is water intolerant and must be performed in anhydrous DMSO. Glycidyl methacrylate is another methacrylating agent that can be used with silk fibroin and is sufficiently stable in aqueous environments to methacrylate silk, although it requires addition in very large excess (Scheme 10).

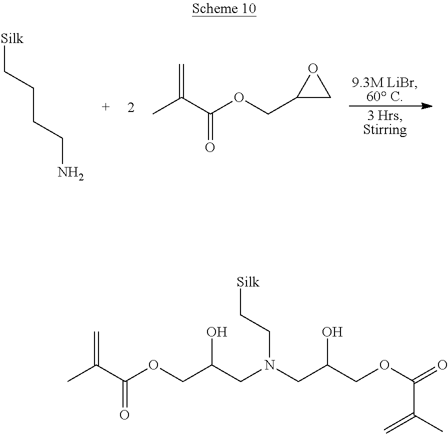

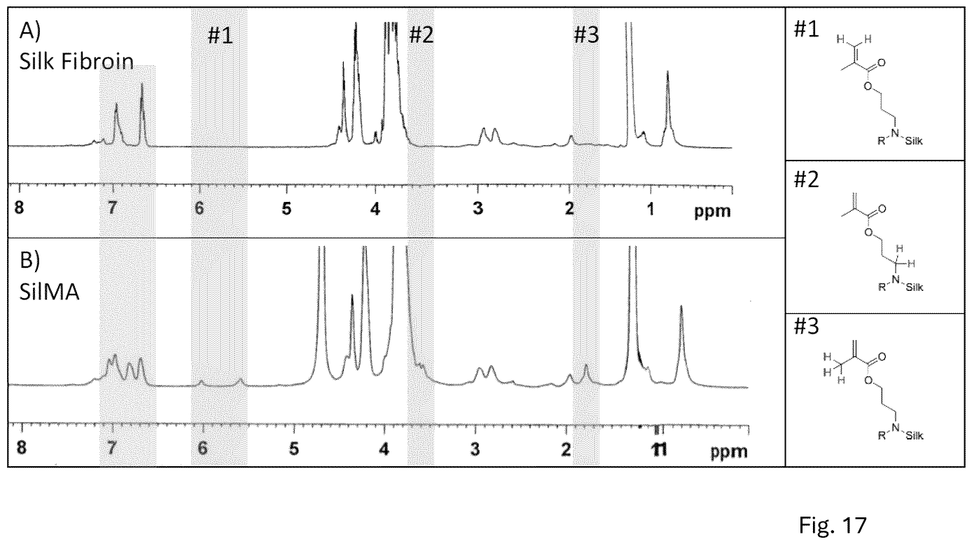

To perform the methacrylation reaction, glycidyl methacrylate is added to silk in lithium bromide solution and the solution heated to 60 C. Afterwards the SilMA can be purified by dialysis in the same manner as ordinary silk. To assess the reaction efficiency, 1H-NMR was performed and compared to unmodified SF (FIG. 17A). The protons on the alkene region of the methacrylate are present at 5.56 and 6.00 PPM in SilMA and absent in unmodified SF. To calculate reaction efficiency we can perform proton integration analysis in the ¹H NMR spectrum. Methacrylate peaks are easily identifiable, thus integrate the protons at 1.75, 5.56, and 6.00 PPM, corresponding to the 3 methyl protons of the methyl group and the cis and trans protons across the terminal alkene respectively (FIG. 17B).

The protons in fibroin are harder to unambiguously assign given the broad overlap between many of the chemical shifts of various amino acids and their side chains. The peaks with the fewest number of overlapping signals are the peaks at 6.5-7.0 PPM that arise from the aromatic peaks in tyrosine, phenylalanine and tryptophan, which intact SF contains 277, 14, and 11 residues of respectively. Taking the average of the number of contributing protons (4 from tyrosine and 5 from phenylalanine and tryptophan) weighted by their numerical prevalence in the sequence gives us an averaged integration value of 4.08 protons responsible for the signal. With our peak integrals identified, we can calculate the degree of methacrylation by summing the integrated signals and dividing them by the number of protons responsible for those signals. This gives us a value of 0.32, which can be then multiplied by the number of amino acid residues (302) to give the total number of methacrylate groups added to a full-length fibroin molecule, which was 96.

The mechanism suggested by Kim et al. asserts that only lysine residues were functionalized, however given that there are only 12 lysine residues per silk molecule, even with 2 methacrylate groups per lysine and the N-terminus, that leaves 70 methacrylate residues unaccounted for. This suggests that the hydroxyl groups on both tyrosine and serine, as well as the carboxylate groups of aspartate and glutamate residues, though not as nucleophilic as lysine were still able to attack the epoxide ring to form ether and ester bonds respectively (Scheme 11). The prevalence of the hydroxyl-bearing (912 per molecule) and carboxyl-bearing (55 per molecule) amino acids can account for the excess functionalization. Finally, the presence of peak-shifts in the aromatic peaks seen in the 6.5-7.0 region also suggests tyrosine participation in the reaction (Scheme 12).

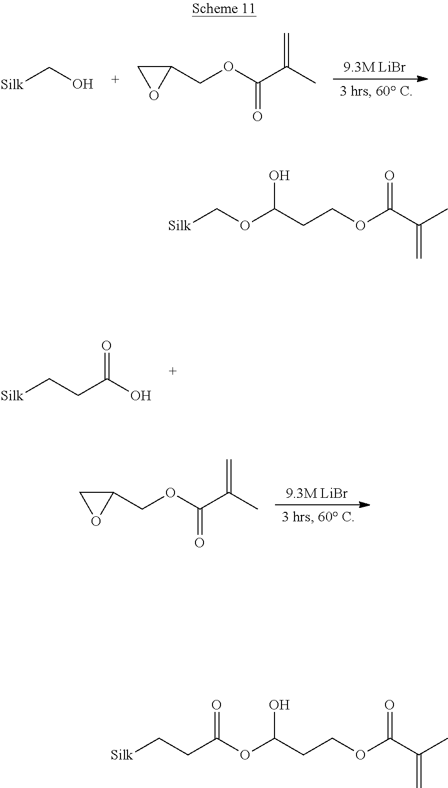

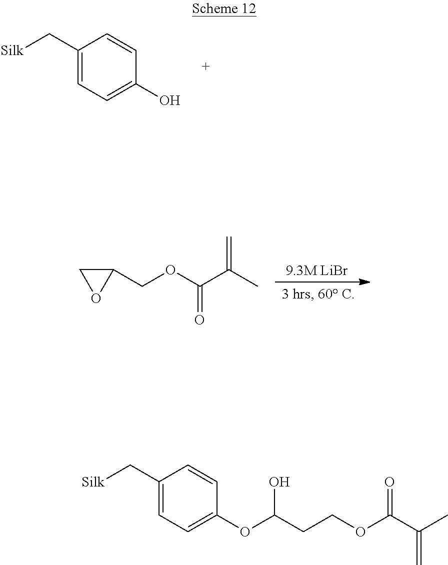

As a secondary test of successful modification, the photoinitiator Lithium phenyl-2,4,6-trimethylbenzoylphosphinate (LAP) was added to a final concentration of 1% w/v to a 5% w/v SilMA solution. When exposed to 365 nm light, this solution quickly gelled indication the methacrylate was covalently bound to the SF.

### SilMA-Bound Pyrazoline Fluorescence

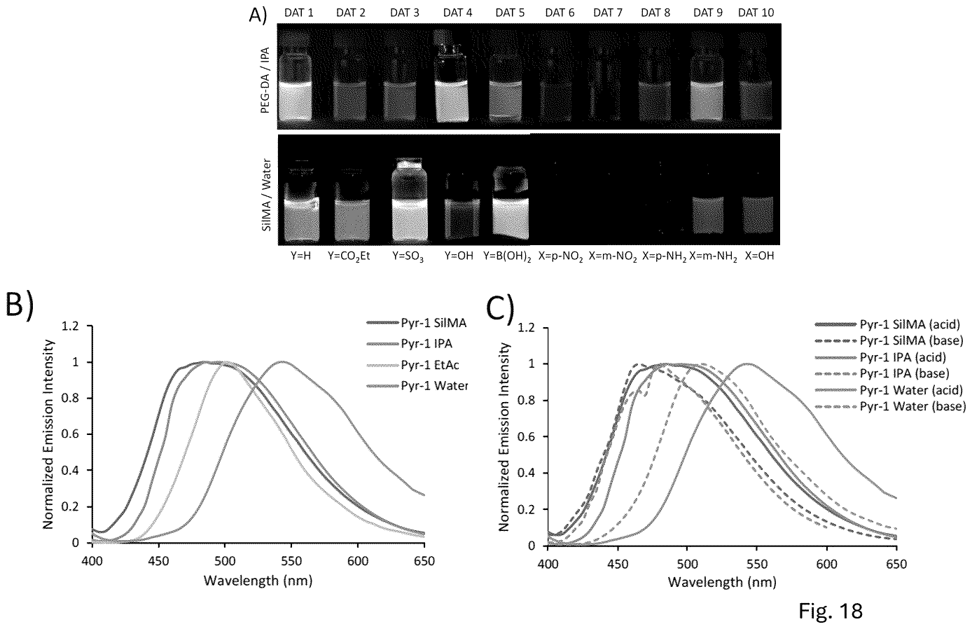

The methacrylate groups in SilMA are also able to participate in the photoclick reaction when mixed with DATs and exposed to 302 nm light, producing Pyrazoline bound to SilMA (Pyr-SilMA). The fluorescent emissions of these fluorescent groups display more similarity to Pyr bound to PEG-DA (Pyr-PEG-DA) dissolved in IPA that Pyr-PEG-DA dissolved in water (FIG. 18A,B). This is counterintuitive as SilMA is always used in an aqueous environment and is in fact insoluble in most organic solvents. Pyr-SilMA also shows acid-base behavior that is more similar to Pyr-PEG-DA in IPA than in water as well (FIG. 18C). The absorption and emission maxima of all Pyr-SilMA derivatives are tabulated on Table 4 and full emission spectra can be found in Appendix A1.4 of Ostrovsky-Snider.

TABLE 4
Functional Group

| Derivative | DAT-1 | DAT-2 | DAT-3 | DAT-4 | DAT-5 | DAT-9 | DAT-10 |
|---|---|---|---|---|---|---|---|
| X | -p-CO2H | -p-CO2H | -p-CO2H | -p-CO2H | -p-CO2H | -m-NH2 | -p-OH |
| Y | —H | —CO2Et | —SO3— | —OH | —B(OH)2 | —H | —H |

| | | | | | | | |
|---|---|---|---|---|---|---|---|
| pH | DAT-1 | DAT-2 | DAT-3 | DAT-4 | DAT-5 | DAT-9 | DAT-10 |

Excitation λmax (nm)

| | | | | | | | |
|---|---|---|---|---|---|---|---|
| 3 | 380 | 370 | 385 | 335 | 370 | 365 | na |
| 10 | 380 | 350 | 375 | 360 | 380 | 375 | 355 |

Emission λmax (nm)

| | | | | | | | |
|---|---|---|---|---|---|---|---|
| 3 | 495 | 460 | 490 | 530 | 505 | 455 | 435 |
| 10 | 465 | 455 | 460 | 530 | 475 | 460 | 435 |

The exact reason for this behavior is not clear, however it may be explained by the hydrophobic shielding of the Pyr cores by the protein. SF naturally exists as a micellar suspension of proteins, with the many hydrophobic domains shielded from the external solvent to minimize unfavorable intermolecular forces. The pyrazoline moieties are themselves more hydrophobic than aqueous silk, and it would be reasonable to assert that the fluorescent core would remain shielded from aqueous solvent by being drawn to the center of the micelles. This shielding would produce effective solvent interactions more similar a semi-polar solvent like IPA rather than a highly polar solvent like water.

### Developing a Size Exclusion Chromatography Method for Silk Fibroin

Chemical characterization of polymers can be very difficult owing to the complex chemical nature of the polymer, internal interactions that create different physiochemical domains on a nano-scale, and the difficulty of processing large molecules in certain techniques. One of the most common ways to assess if polymer grafting chemistry was successful is the use of size-exclusion chromatography (SEC). This technique can be applied to polymers up the MDa size ranges and can be associated with UV-Vis detectors to determine if the grafted moiety was actually bound to the protein or merely co-dissolved in solution. Here I wanted to use SEC to characterize silk but ran into a multitude of difficulties.

Ideally silk could be dissolved in water or an aqueous buffer for the SEC analysis, however when water is used as the mobile phase agglomeration is immediately apparent. This is due to two factors, the high shear-sensitivity of SF and the micellular nature of aqueous SF. At low shear SF behaves as a Newtonian liquid, however when silk exceeds its sheer threshold it becomes shear-thinning. This shear also induces beta-sheet formation which causes intermolecular agglomeration. To combat both of these factors, a denaturant must be used in the mobile phase. Several common denaturants were examined to determine their suitability, namely SDS, LiBr and urea. Unfortunately, the literature that shows SDS tends to induce agglomeration in silk, while silk dissolved in LiBr still shows susceptibility to shear. Urea-based mobile phases on the other hand has been documented as a mobile phase denaturant for SF before and has been shown to stabilize the fibroin against shear-induced aggregation during SEC.

The necessity of using urea adds a further complication to the SEC method by limiting the available selection of protein standards. Many globular proteins that are commonly used for SEC calibration have polymeric quaternary structure, for example bovine immunoglobulin G has 6 substituents (being a dimer of trimers) that held together by a combination of disulfide bonds and intermolecular forces. The urea in the solution destroys those intermolecular forces and leaves the disulfide bonds susceptible to sheer-based breakages. To limit this as much as possible, monomeric peptides were chosen as often as possible. The proteins of bovine serum albumin, gallus ovalbumin, and equine myoglobin were chosen. Cytidine was selected as a small molecule standard due to its similar polarity and h-bond potential to proteins, as well as it's strong absorption at 280 nm.

However, due to the size of native SF, a high Mw standard was needed, but unfortunately very few monomeric peptides exceed the Mw of fibroin and those that do were exceptionally expensive and thus unsuitable for calibrants. Bovine thyroglobulin was thus selected for a high Mw standard despite being dimeric. It is a homodimer and in denaturing solution will thus dissociate to form two identical denatured proteins. This avoids the multiple overlapping peaks as seen in immunoglobulin G and allows us to use it as a high molecular weight standard. This is unfortunately the largest commercially available protein suitable for use as a standard. Thus, the upper masses of silk fibroin will elute outside the upper end of the calibration curve and we must rely on the continued linearity of the calibration curve above the highest standard. To ameliorate this problem and simultaneously mitigate spontaneous gelation, long boil-time silk fibroin will be used in grafting experiments when an increase in molecular weight is one of the expected indicators of a successful grafting reaction.

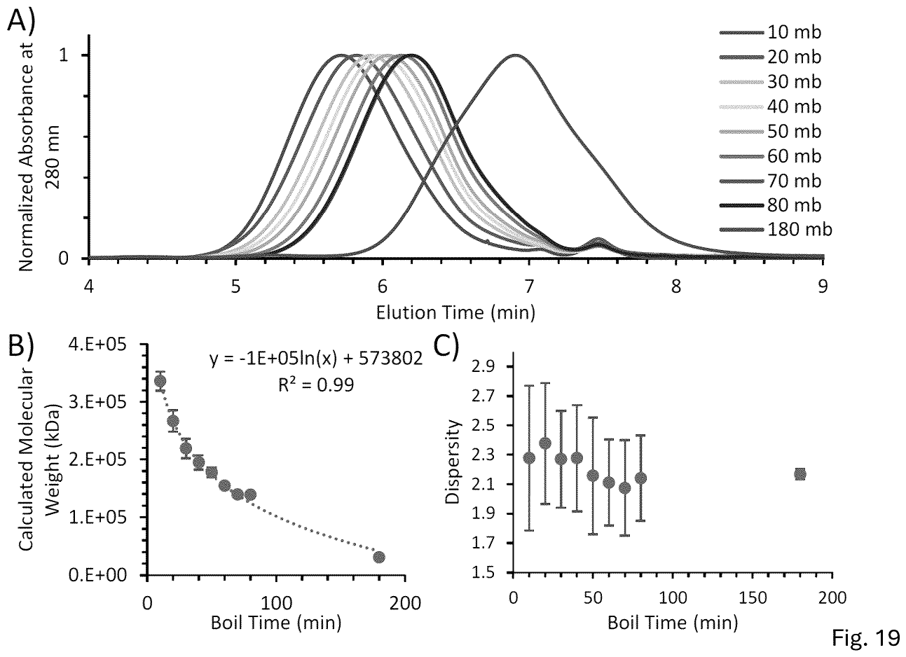

It has been well established that increasing the boil time during degumming decreases the molecular weight of the SF that is recovered at the end of the dialysis process. Silk was boiled for various times between 10 and 180 minutes and each batch was analyzed by SEC analysis. The silk had peak elution times between 5.72 and 6.90 minutes corresponding to molecular weights of 335 kDa and 31 kDa respectively. This agrees well with literature values estimated by SDS-PAGE. When molecular weight is plotted against boil time a clear trend emerges (FIG. 19). Short boil times yield molecular weights near to the full-size fibroin chain, but also clearly show degradation has already begun. Initial loss of average molecular weight it rapid as large chains are broken, but slows as the hydrolysis of smaller chains has less of an impact on the average molecular weight (FIG. 19B). This is also indicated in dispersity as it has already reached a near-static level after 10 minutes of boiling (FIG. 19C).

### Attempts to Conjugate DATs to Fibroin Via Carbodiimide Reactions

The usefulness and facility of photoclick-able silk would have been greatly augmented by the ability to covalently bind the DAT molecule to the fibroin backbone and allow it to be used for conjugation later. To this end, several synthetic mechanisms were attempted to link the DAT to the silk fibroin using carbodiimide chemistry or an azo-dye reaction. The success or failure to conjugate these molecules to silk was judged by both fluorimetry to detect the evolution of fluorescent pyrazoline adducts and size-exclusion chromatography to search for the shift in molecular weight from the binding of alkene-bearing polymers like PEG-DA.

Initial attempts to conjugate a DAT molecule to SF were executed with carbodiimide chemistry (Scheme 13). There are several lysine residues and the N-terminus which would be viable targets for amide-bond formation with a carboxyl derivative. So, to this end DAT-1 was treated with EDC/NHS to create a reactive NHS ester, then added to silk fibroin and mixed overnight at room temperature. This was purified by dialysis and the resulting product exhibited some fluorescence. However when later analyzed by SEC it was found there was no significant increase in absorption at 300 nm as would be expected from a DAT conjugate, nor was there a molecular weight shift seen when mixed with PEG-DA and exposed to 365 nm light.

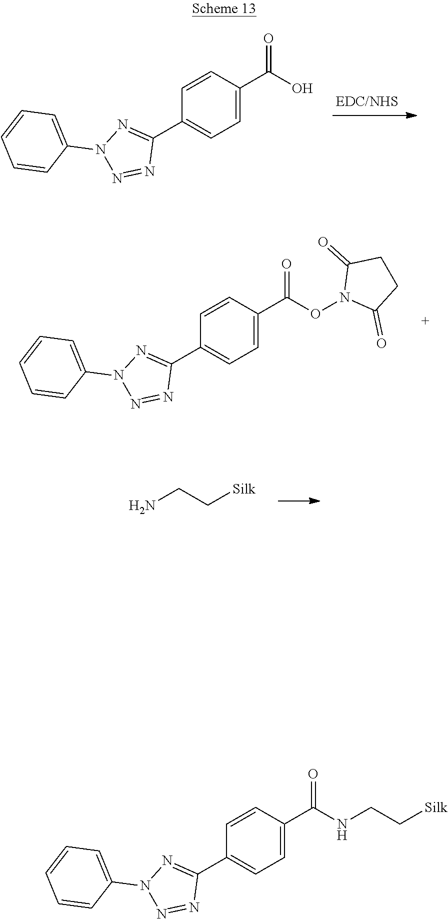

An alternate strategy would be the formation of amide bonds between native carboxyl groups in the SF and a amino derivative of DAT like DAT-8 or DAT-9 (Scheme 14). These were both attempted but similarly failed to produce a shift in molecular weight when photoclicked to PEG-DA. During both of the carbodiimide experiments there were significant difficulties in dissolving the DAT into the solution and transferring to the reaction buffer would precipitate the DAT. While the DATs were poorly soluble in water, they are easily dissolved in DMSO, however SF is not very DMSO soluble. The solubility of SF in DMSO can be improved by carboxylation, which would also have the added benefit of increasing the number of reaction sites with which amine bearing DATs can react.

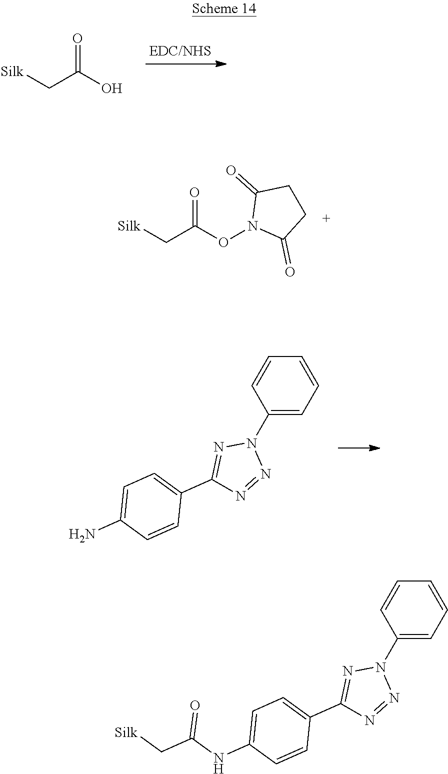

The previous reaction relied on the natural abundance of carboxyl groups in SF, however the number of said groups could be augmented chemically by the reaction of silk with chloroacetic acid or acetic anhydride. The former reaction is possible in water, however the chloroacetic acid rapidly degrades the backbone of the SF, and so there is a limit to the length of time the reaction can be allowed to run (Scheme 15A). The latter reaction cannot be performed in water, as the acetic anhydride is too susceptible to hydrolysis by water to be useful for carboxylating the SF. Instead, the SF is dissolved in the deep eutectic solvent butyl imidazolium chloride (BMIM-Cl) and diluted with dimethyl formamide to allow for easier stirring and handling (Scheme 15B). The advantage here is that the silk will not be hydrolyzed in the reaction process, but transitioning the silk out of the deep eutectic solvent inevitable precipitated it and thus requires it to be redissolved in LiBr solution then dialyzed to remove the LiBr.

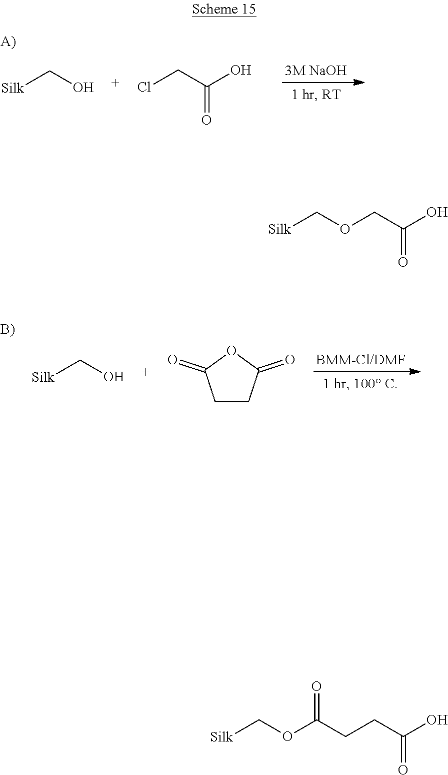

Both carboxylation reactions were performed, and the resulting SF was analyzed by SEC and NMR. Post-reaction molecular weights showed that both reaction conditions had degraded the molecular weight, however the chloroacetic acid reaction had degraded it far more (see figures in Ostrovsky-Snider). NMR analysis of both showed new peaks emerging with the chloroacetic acid reaction yielding new peaks at 3.25 and 3.44 PPM, although which corresponds to the new protons is difficult to determine. The silk carboxylated in by succinic anhydride showed distinct new signals at 2.4 and 2.5 PPM corresponding to the 4 protons between the carbonyls (see figures in Ostrovsky-Snider). Given that the succinic anhydride reaction was gentler on the SF and clearly produced a higher degree of functionalization, the SF resulting from the carboxylation reaction with succinic anhydride was used for further conjugation reactions.

Carboxylated silk was dissolved in DMSO and instead of EDC, HBTU was used as it is better suited for organic solvents like DMSO. In an attempt to aid the reaction, N,N-disopropylamine was added as a non-nucleophilic base. The reaction was attempted with DAT-8, which remained fully dissolved over the course of the reaction. Unfortunately, this too failed to achieve any substantial reaction with the SF. The SF was purified after the attempted coupling, and was analyzed by SEC. No increase in molecular weight was observed, instead indicating a slight loss in molecular weight. The absorbance at 330 nm was slightly increased but ultimately it was not determined to be a significant enough increase to continue pursuing this reaction.

### Attempts to Conjugate DATs to Fibroin Via Azo Dye Reaction

The repeated failure to create an amide bond with carbodiimide chemistry caused me to abandon the reaction scheme for other avenues of conjugation. Amino derivatives DAT-8 and DAT-9 both had benzylamines and so were suitable for turning into diazonium salts. The salts form azo-dye bonds with tyrosines in silk, which are far more abundant than either lysine or glutamate/aspartate residues. Diazoniums are formed by reacting benzyl amines with nitrous acid, however they are generally unstable and must be formed in situ.

To make DAT-diazonium, DAT-8 and DAT-9 were dissolved in acidic ethanol, cooled and sodium nitrite was added to form the nitrous acid, which subsequently formed the diazonium. The reaction occurred quickly and thanks to the electron-withdrawing nature of the tetrazole, there was no detectable self-reactivity during diazonium formation. This product was quickly added to SF in BBS (pH 10.5), and the fibroin turned a vivid red with seconds, thereby showing the reaction was successful. The reaction was allowed to continue for 1 hour until all of the diazonium was certain to have been consumed. UV-Vis analysis showed the azo-dye silk had developed a broad absorption peak centered approximately at 330 nm.

The product was quickly purified by use of a desalting column and the photoclick reaction was attempted. The azo-dyed SF was mixed with PEG-DA and exposed to UV light, however no fluorescent product was formed. This was unfortunate but not unexpected as the fluorescence may have been masked by the now deep red silk fibroin, so it too was analyzed by SEC. The SEC clearly shows the diazonium reaction was successful due to the increased absorbance at 330 nm of the azo-dye, however there was no significant post-UV exposure increase in molecular weight. This indicates that while the conjugation reaction was successful, the chromophore produced by the reaction completely masked the DAT from the UV light and defeated its ability to participate in the photoclick reaction.

### Gomberg-Bachmann Reactions

The successful creation of a DAT-diazonium, while not directly usable, enabled the prospect of using a Gomberg-Bachmann reaction to create a direct biphenyl linkage to the aromatic residues in SF. The Gomberg-Bachmann reaction exploits the tendency of diazoniums to undergo elimination reactions to produce molecular nitrogen, thus leaving a transient reaction site for substitution to occur. With the addition of a transition metal catalyst, the elimination produces an aryl radical as opposed to a cation, which improves the stability of the intermediate and forms a catalytic cycle. This reaction mechanism would be superior to carbodiimide chemistry for silk fibroin owing to the high number of tyrosines present in the silk backbone.

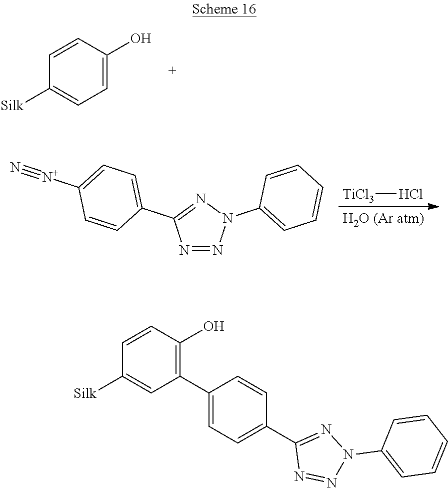

In an attempt to increase the reactivity of the DAT-diazonium in elimination reactions, the triflate counter ion was introduced instead of chloride. Studies by Filimonov et al. (V. D. Filimonov et al., “Synthesis, Structure, and Synthetic Potential of Arenediazonium Trifluoromethanesulfonates as Stable and Safe Diazonium Salts,” European Journal of Organic Chemistry, vol. 2019, no. 4, pp. 665-674, 2019, doi: 10.1002/ejoc.201800887, which is incorporated herein in its entirety by reference for all purposes) showed that diazonium triflates would spontaneously undergo elimination reaction, which is not seen with any other counterion. Synthesizing DAT-diazonium triflate required dissolving the amino-DAT in acetic acid and chilling it to 14 C which is just above the freezing point of the acid. Once cooled triflic acid was added, the amyl nitrite was added as a nitrite source. The reaction was monitored by TLC, and when complete the diazonium salt was precipitated in ethyl ether. The triflate counterion stabilizes the diazonium sufficiently that the solid is not explosive, unlike halide salts which spontaneously explode upon precipitation. The resulting triflate salt was a light brown solid that was soluble in both water and organic solvents like acetone, IPA and DCM. When mixed with SF in BBS (pH 10.5) it formed the azo dye and turned the fibroin deep red within seconds of addition.

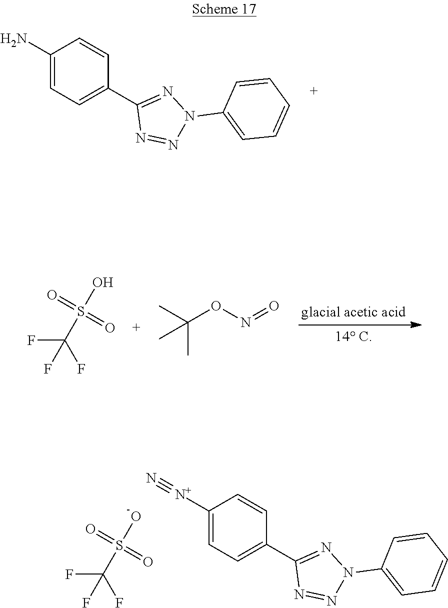

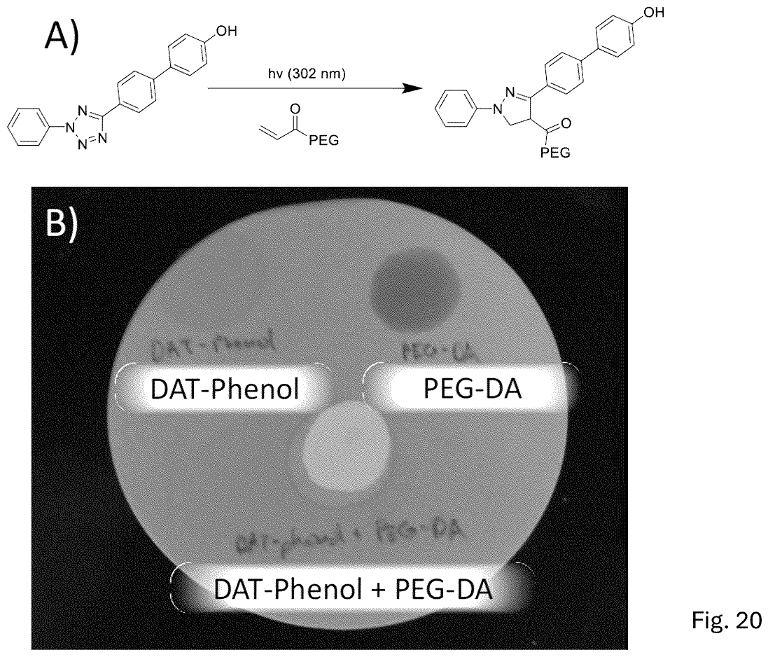

The reaction was first attempted on a small molecule substrate to be sure the mechanism worked before introducing the complex matrix of SF, so phenol was chosen as a stand in for the tyrosine side chain. The titanium catalyst is intolerant to oxygen, so the reaction was performed in a sealed round-bottom flask purged with argon, while all the solutions were sparged with argon prior to addition to the reaction vessel. After the reaction was complete the product was purified by extraction with ethyl acetate. The purified product was mixed with PEG-DA and produced a blue-fluorescent compound after UV exposure (FIG. 20).

A portion of the product was reacted with the small alkene-bearing molecule N,N-isopropylacrylamide (NIPAM), which also produced a blue-green fluorescent product. LC-MS analysis of both the raw product and the NIPAM-reacted product revealed a molecular ion of mass 314 Da in the un-clicked product and 399 Da in the photoclicked-product, corresponding exactly to the expected masses of the DAT-Phenol precursor and the corresponding pyrazoline product. A mass of 222 Da was also observed, corresponding to side product arising from diazonium elimination followed by hydrogen substitution. Double peaks for both product both before and after the photoclick is likely due to an ortho-substituted product rather than the para-substituted product shown. The ortho product is likely in lower abundance due to the steric hinderance of the hydroxyl group. This demonstrated both that the Gomberg-Bachmann reaction was possible with a DAT-diazonium and that the product of the reaction remained UV-sensitive and could still participate in the photoclick reaction.

For this reaction to be attempted on silk fibroin, the principal hurdle of solvent compatibility would need to be overcome. The titanium catalyst is dissolved in 3 M hydrochloric acid, which makes it incompatible with SF as being at or below the isoelectric point (pH 4) causes silk to precipitate out of solution. Additions of various chaotropic salts such as calcium chloride and lithium bromide were attempted but were insufficient to keep the silk stable in the reaction. The reaction supernatant was found to still contain a small quantity of soluble SF, which was purified by filtration and desalting. The solution was mixed with a large excess of PEG-DA and exposed to 302 nm light, and a blue-green fluorescence rapidly developed. Both unreacted and reacted SF was analyzed by SEC, revealing the unreacted SF had acquired a substantial absorbance at 330 nm, indicative of a bound DAT. Furthermore, the mass and absorbance at 330 nm both increased after UV exposure indicating that PEG-DA has been grafted and that bound pyrazolines have been formed.

While the arylation reaction was ultimately successful, the harsh reaction conditions made purification very difficult, as evidenced by the large number of contaminants in the SEC chromatograph. Yields were ultimately very low, as much of the silk was lost when trying to remove the titanium precipitants. Further work is needed to make this functionalization a viable mechanism for efficiently functionalizing SF.

### Conclusion

Here we demonstrated successful synthesis of SilMA from SF and glycidyl methacrylate. Integration of the proton signals in the NMR suggests that more methacrylate groups were grafted onto silk fibroin than available lysines can account for. Given the abundance of tyrosine and the observed peak shifts of the aromatic tyrosine peaks, I postulate that the hydroxyl group on tyrosine is the principal nucleophile responsible for the methacrylation of silk.

The SilMA product was also able to react with the various DATs to produce pyrazoline fluorescent products. These Pyr-SilMA behaved most similarly to Pyr-PEG-Das dissolved in IPA despite the SilMA being dissolved in water. I conjectured that this was the result of the pyrazoline core being hidden inside SilMA micelles, thus partially shielding them from the solvent.

Various methods were attempted to graft DATs onto SF, using carbodiimide, diazonium and arylation chemistry. The carbodiimide reactions were universally unsuccessful, despite the enrichment of carboxyl moieties on SF. The azo-dye reactions proved successful in grafting but destroyed the photoreactivity of the DATs. Gomberg-Bachmann arylation was ultimately successful at grafting DATs to SF, and their photoreactivity was preserved. However, yields of SF were very low due to difficulty in stabilizing the silk during the reaction and purifying it afterwards.

### Future Directions

Future work towards synthesizing silk fibroin that has photoclickable DAT covalently bound to the SF can begin by expanding on the few successes that I was able to achieve here. The Gomberg-Bachmann reaction is able to directly link the aryl groups of the DAT to the highly abundant tyrosine residues in silk fibroin, albeit at the cost of making a highly unstable solution that is prone to spontaneous gelation. This is likely due to the hydrophobic collapse of the SF, and has been seen in other reactions that functionalize the tyrosine residue, particularly the azo-dye reaction. This however also provides a simple mechanism for alleviating this problem: introduce a stable formal charge to the DAT molecule. Azo-dyed silk with sulfanilic acid exhibits similar solution stability to unmodified fibroin, and the same strategy of sulfation can be used here. Sulfated DATs were demonstrated to remain photoactive in Chapter 2 and increases the water solubility of DAT, which is the primary solvent for Gomberg-Bachmann arylation.

Another avenue to explore for decorating SF with DAT is the synthesis of highly-reactive derivatives. Such possibilities include the conversion of the amine variants into isothiocyanates, or the conversion of the carboxylate derivatives into acid chlorides that could be reacted to SF using a deep-eutectic solvent system such as those based on imidazolium, which readily dissolve SF.

### Tattoos and Patterned Fluorescence

### Overview

The photoclickable DATs produced in the previous section displayed a variety of fluorescent emission colors that could be exploited to produce photopatterned fluorescence, which have diverse utility in the fields of anti-counterfeiting and medicine. Films of SilMA impregnated with DAT dye precursors are well suited to patterning by the use of photomasks in a pseudo-lithographic process. The patterns of light and shade will be reproduced in negative on the substrate, where areas exposed to 302 nm light will become fluorescent while those shaded from the light will stay non-fluorescent, making it akin to a negative photolithographic process. This process can be extended to multi-layer fluorescent films by adding subsequent layers of SilMA and reacting that with DAT dye precursors to produce an independently patterned layer on top of the previous. The use of separate dyes on each layer enable them to act independently and have different responses to environmental conditions. The use of SF and PEG-DA composites enables the use of several other DATs that were excessively quenched by the SilMA matrix but have visible emission in the composite.

Tattoo inks could also be plausibly fabricated given that both SilMA and the DATs have been shown to be highly biocompatible. Microparticles of SilMA were created via a coaxial needle co-flow system with PVA and were subsequently loaded with DAT-1 dye precursor. These particles were able to be used as a traditional tattoo ink and showed improved pattern retention compared to chemically identical inks in an aqueous polymer format. These inks were further able to be patterned after implantation by UV activation through a photomask in both agarose phantoms and explanted porcine skin, yielding visible fluorescent patterns in both substrates.

While traditional tattoo inks must be patterned during implantation, the post-implantation patterning of photoclick tattoos enables a simplification of the implantation procedure with microneedle arrays. These arrays can implant the unreacted photoclick ink indiscriminately over a broad area both quickly and in a less-painful manner. To make such an array, SilMA microparticles loaded with DAT-1 dye were suspended in unmodified fibroin and cast in a PDMS microneedle mold. The resulting microneedles were demonstrated to successfully deliver their cargo into an agarose imaging phantom, however the spacing between the needles limited the resolution and brightness of patterns that could be achieved with a single layer. To improve the dye density multiple layers could be applied on top of each other to fill in the gaps left by previous layers.

Finally the DAT-loaded SilMA microparticles were tested for cytocompatibility to ensure no processes in their fabrication introduced cytotoxic elements. The small molecule dye was first tested for cytotoxicity in a 2D environment by incubating human dermal fibroblasts (hDFs) with the DAT dye for 24 hours and observing both their cellular morphology through microscopy and metabolic rate through the MTT assay. The full microparticles and were then tested by implantation into a 3D cell culture matrix, composed of skin mimicking SF sponge loaded with hDFs. The 3D matrix was incubated for 7 days with the dye particles and cytotoxicity was assessed by cellular morphology and MTT assay. The cellular morphology could not be seen with simple optical microscopy, so the cell's cytoskeletal structure was stained via immunohistochemical dyes and imaged under a z-resolved fluorescent microscope.

### Background

Patternable fluorescence has a wide utility in anti-counterfeiting, being used for selectively visible markers, and physically unclonable functions (PUFs). Selectively visible labels only appear under certain conditions, such as illumination with UV-light and therefore present a challenge-response pair that is more difficult to copy than a permanently visible pattern. This technique has already been used to prevent counterfeiting of currency and preserving the chain of ownership of high value items. This can be extended a step further with PUFs, which are in essence unique identifiers that are exceptionally difficult to copy owing to their inherent complexity. These are fabricated with inherent randomness as a core feature, as each new PUF will be a unique identifier traceable at any point in its lifetime. Fluorescent PUFs add yet another layer of security as the challenge and response allows multi dynamic outputs, further complicating duplication.

Tattoos have been a common aesthetic practice by many human cultures for all recorded history and into prehistory as well. While the materials have changed significantly over the course of the millennia, the fundamental process has changed little. To create a tattoo, a small sharp needle is coated with a particulate ink such as metal oxides like TiO2, carbon particles, or polymer bound azo dyes. This ink-laden needle is pushed into the skin far enough that it deposits the ink in the dermis but should not penetrate into the subcutaneous tissues. This ink then remains visible by its proximity to the surface of the skin but is sufficiently deep that it is not lost during the desquamation of the epidermis. The particulate nature of the dye is similarly crucial to the longevity of the marking. Small molecular dyes and sub 100 nm particles would readily diffuse in the interstitial fluid and be recycled via the lymphatic system, so large particles (˜800 nm or larger) are needed to stay entrapped within the extracellular matrix of the dermis, the lower layer of the skin.

The skin is the single largest organ of the human body and performs numerous biological functions, chief among them being a nearly impassible barrier to prevent loss of water and the ingress of harmful pathogens and environmental chemicals. In humans the skin ranges from 2 to 7 mm thick depending on the location on the body and is composed of two primary layers: the dermis (2-6 mm thick) and the epidermis (0.1-1.6 mm thick). The epidermis is the outermost layer that is in contact with the outside environment and is composed of keratinocytes that replicate in the basale stratum and are differentiated as they move outward until they become the enucleated corneocytes of the outermost stratum corneum. This stratum is constituted by multiple layers of interlocking corneocytes with highly crosslinked cytoskeletons and bound together by a keratinous extracellular matrix and is typically 10-30 μm thick. The constant abrasion and damage to this tissue requires constant replacement of the outer layers, which is accomplished by continuous generation and differentiation of keratinocytes and desquamation of the outer most layers. The full process from differentiation to desquamation takes about 14-28 days and thus any damage or marking to the epidermis will be lost in that time frame.

Underneath the epidermis is the dermis, which contains the hair follicles and exocrine glands of the skin as well as the blood and lymphatic vessels that sustain them. This layer is also responsible for providing the mechanical toughness and elasticity of the skin and accomplishes this by a strong extracellular matrix composed of connective proteins like collagen as well as glycans such as hyaluronan. Crucially for our purposes, this layer is well hydrated by interstitial fluid and is readily accessible by immune cells such as neutrophils and macrophages. This means that tattoo inks must be immune compatible, however their size helps mask some of their systemic effects. Immune capture of tattoo ink particles is driven entirely by macrophages that engulf the pigment and store it in large vacuole. This isolation from the rest of the body is responsible for the moderating effect on the intrinsic toxicity of tattoos, as heavy metals like cobalt, zinc, cadmium, and mercury were commonly used but had greatly blunted systemic effects thanks to their isolation.

### Methods and Materials

Methods of the synthesis of DAT can be found above. Methods for the isolation of silk fibroin and synthesis of silk fibroin methacrylate can be found above.

### Microscopy and Imaging

### Immunohistochemistry

Cell-laden 3D scaffolds were fixed in a 2% formalin solution in PBS for 2 hours with gentle oscillatory mixing. All subsequent washing steps were performed for 30 minutes with oscillatory mixing with the indicated solution. The formalin solution was then decanted, and the scaffolds were washed 3 times with DI water. The cell membranes were permeabilized by washing the scaffolds with acetone solution (50% v/v acetone in 1×PBS) for 30 minutes. The samples were then washed 3 times with PBS-T (1×PBS, 1% Triton X-100), then blocked with 1 mg/mL BSA in PBS-T (1×PBS, 0.1% Triton X-100). Actin was stained with phalloidin-488 (Invitrogen A12379) for 1 hour, then washed 3 times with 1×PBS. Nuclei were stained with DAPI (Invitrogen D3571) for 30 minutes then washed with PBS 3 times. Scaffolds were stored in 1×PBS at 4 C until imaged.

### Z-Resolved Fluorescence Microscopy

Fixed and stained samples as well as tattooed imaging phantoms were imaged with a Keyence BZ-X710 all-in-one fluorescence microscope. The DAPI filter channel (ex/em 355/450) was used to image both the DAPI stain, fluorescent pyrazoline-laden microparticles, and aqueous SilMA-pyrazoline tattoo inks. The GFP channel (ex/em 488/510) was used to image the phalloidin-stained actin.

### Multispectral Imaging

Multispectral imaging was performed with a Nuance FX multispectral imaging system with a Canon macro lens and illuminated by 365 nm light. Exposure times were varied by sample and were selected by automatic exposure timing in the acquisition software. Images were processed using Nuance software and spectral elements were picked by hand from at least 20 sample locations. Raw processed spectra had large discontinuities introduced by the internal monochromator, which were removed with a custom algorithm.

### Bright Field Transmission Microscopy and Image Processing

Images were taken with a Motic AE2000 inverted microscope in bright field transmission through the bottom of transparent 96 well plates. Images from 2D cell culture were processed in GNU Image Manipulation Program V 2.10.12. Contrast was enhanced by changing light and dark levels to maximize the dynamic range of the image. The illumination artifacts were removed by creating a duplicate image on a separate layer and blurring it with a Gaussian blur strength 12.0. This layer was placed below the normal layer, and the two layers were mixed in the “Grain Extract” mixing mode. This process inverts the colors, so the color was inverted again to restore the original orientation. The raw, unaltered images are available in Appendix II.

### Microbiological Assays

### 2D Metabolic Toxicity Assay

Primary human dermal fibroblasts were seeded in 96 well plates at 5000 cells per well and cultured for 3 days in growth media (1×DMEM+GlutaMax, 10% fetal bovine serum, 1× streptomycin/amphotericin) until fully confluent. No wells on the outside perimeter of the plate were used for cell culture, these were filled with sterile 1×DPBS to mitigate evaporative effects. Media was premixed with DAT at various concentration and 1% v/v DMSO, positive control was mixed with 1% Triton X-100, and vehicle control was mixed with 1% DMSO. Cells were incubated for 24 hours then media was replaced with 100 μL of MTT-media (90% growth media, 10% Biotium MTT assay reagent (#30006)) per well then incubated for a further 4 hours. After 4 hours the formazan product was solubilized by addition of 200 μL of DMSO per well. Results were obtained by measuring UV-Vis absorbance at 570 nm and 630 nm. Background was corrected according to manufacturer instructions by subtracting the absorbance at 630 nm from the absorbance from 570 nm. Metabolic rates were normalized to controls by subtracting the absorbance from cell-free wells to control for media absorbance then dividing by the absorbance of the negative control (media only) to get a fractional cell metabolic rate.

### 3D Metabolic Toxicity Assay

Silk fibroin solution (5% w/w) was pipetted into a 24 well plate (1.5 mL per well) and frozen at −20 C. After 24 hours the plate was moved to −80 C and left for another 24 hours, then lyophilized for 48 hours. The dry scaffold was autoclaved (121 C, 4 Barr) for 40 minutes to fully water-vapor-anneal them, then removed and soaked in deionized water. The wet sponges were cut to 2 mm thickness with a surgical blade and constant depth guide, being sure to keep the lower face of the scaffold that had frozen in contact with the plate, then cut to 7 mm in diameter with a biopsy punch. The wet sponges were autoclaved again for sterility then seeded with human dermal fibroblasts. Human dermal fibroblasts were expanded, harvested and pelleted at 300 RCF. The pellet was resuspended in sufficient ice-cold matrix solution (2×DMEM, 0.1% collagen (type I)) to achieve a concentration of 200k cells per mL. Onto each scaffold was deposited 100 μL of matrix solution and they were placed in incubation at 37 C, 100% humidity for 30 minutes to allow the collagen to crosslink. Scaffolds were placed in 24 well plates with 2 mL of growth media per well and incubated for 24 hours. Scaffolds were then tattooed by injecting 1 μL of ‘ink’ into the center and 6 location around the edge of the scaffold. Positive control was 1 μL of Triton X-100, negative control was 1×DPBS, vehicle control was 1% w/v SilMA microparticles in 1×DPBS, and experimental inks were DAT-loaded microparticles at 1% w/v in 1×DPBS. Scaffolds were incubated for 7 days with media changed after 1-, 3-, and 5-days post tattooing. On day 7 the media was replaced with MTT-media and incubated with oscillatory mixing for 4 hours at 37 C. Afterwards the sponges were washed for 20 minutes with DPBS, then manually divided by cutting them into ˜3 mm3 pieces with sterile scissors. The pieces were immersed in 1 mL of DMSO and the formazan was extracted for 1 hour by oscillatory mixing at 37 C. An aliquot (100 μL) of the formazan laden DMSO was transferred to 96 well plate and measured by UV-Vis absorbance at 570 nm and 630 nm. Background was corrected according to manufacturer instructions by subtracting the absorbance at 630 nm from the absorbance from 570 nm. Metabolic rates were normalized to controls by dividing by the absorbance of the negative control (DPBS) to get a fractional cell metabolic rate.

### Microparticle Fabrication and Loading

A coaxial needle was used with a 16-gauge outer needle and a 26-gauge inner needle and both connected to independent syringe pumps. The continuous phase (5% w/w polyvinyl alcohol, 30-70 kDa) was pumped through the outer needle at 4 mL/hour, while the inner phase (5% w/w SilMA) was pumped through the inner needle at 0.4 mL/hour. The solution was collected in a beaker, then allowed to dry on a PDMS (Sylgard 184) mat in a fume hood until fully dry. The solution dried to a PVA film, which was redissolved in deionized water by vortexing and bath ultrasonication. The microparticles were pelleted by centrifugation (11,000 RCF, FX6100, Beckman Coulter Allegra X-12 centrifuge) for 10 min, then the supernatant was decanted. The particles were resuspended in deionized water then pelleted by centrifugation and the supernatant decanted. This was repeated twice more to remove all PVA from solution. If the particles were to be loaded with DAT dye, the pelleted particles were resuspended in dye solution (50 mg/mL in DMSO) and gently mixed by oscillatory mixing for 1 hour in a sealed centrifuge tube. The loaded particles were pelleted by centrifugation, then dye solution was decanted, and the particles and remaining free dye was resuspended in isopropyl alcohol. The process of resuspension, pelleting and decanting was carried 3 more times with deionized water. Microparticles were stored at a concentration of 100 mg/mL (total solids) in water at 4 C until used.

### Bulk Material Fabrication

### Microneedle Array Fabrication

A microneedle array master was provided by Vaxess, which was a lxii grid of 800 μm long needles, 400 μm diameter at the base and spaced 800 μm apart center to center. A PDMS secondary mold was made from this master by casting Sylgard 184 PDMS (10:1 monomer to curing agent ratio) over the mold, degassing under vacuum for 1 hour, then cure at 60 C for 3 hours. Dye solution was made by mixing DAT-loaded SilMA microparticles to a final concentration of 1% w/v in 5% unmodified silk fibroin. Dye solution (100 μL) was pipetted onto the surface of the PDMS secondary mold and was forced into the needles by centrifugation at 2500 RCF in a SX4875 swinging bucket rotor. Another 100 μL of dye solution was added to the needles and allowed to dry at room temperature. Finally, 200 μL of unmodified silk solution was cast onto the microneedles to reinforce the base of the array. The array was manually demolded and stored in the dark at room temperature until use.

### Multilayer Fluorescent Pattern Fabrication

A glass coverslip (22×22 mm) was placed onto the vacuum chuck of a Laurell WS-650 spin coater. Onto the surface was pipetted 100 μL of silk solution (7.0% w/w silk fibroin, 0.7% w/w polyethylene glycol diacrylate), which was manually spread to cover the entire surface. The solution was spun with a program that had a 5 sec spread phase (500 RPM, 250 RPM/sec), followed by a 60 sec thinning phase (1500 RPM, 500 RPM/sec) then a 15 second evaporation phase (4000 RPM, 500 RPM/sec). The sample was then water vapor annealed by being placed in a Memmert UN 30 Plus humidity-controlled oven at 45 C and 90% relative humidity. A timer was started once the oven had reached 90% humidity after then drop due to sample addition and allowed to sit in the oven for 5.0 minutes. Once removed the sample was loaded onto the vacuum chuck again and 30 μL of DAT solution (5 mg/mL DAT in isopropyl alcohol) was dropped onto the surface. A combined spreading/evaporation program was run with a single 20 second phase (4000 RPM, 250 RPM/sec). The sample was then covered by a photomask and exposed to 302 nm UV light (Labortechnik UVLMS-38 8 Watt UV multiwavelength lamp) to induce the photoclick reaction. If a multi-emitter film was to be fabricated, then a PDMS isolation layer must be applied if the lower layer is composed of SF/PEG-DA. To form the isolation layer, Sylgard 184 monomer was mixed with curing agent in a 10:1 ratio and 500 μL was applied to the glass slide by spin coating (5 sec, 500 RPM spread, 30 sec 5000 RPM thinning, 500 RPM/sec acceleration). This was cured by heating the slide on a 80 C hotplate for 30 minutes. The subsequent layer could then be applied in an identical manner to the first. If SilMA was used as the lower layer, the isolation layer is not needed and the second layer can be applied directly.

### Results and Discussion

### Patterned Fluorescence

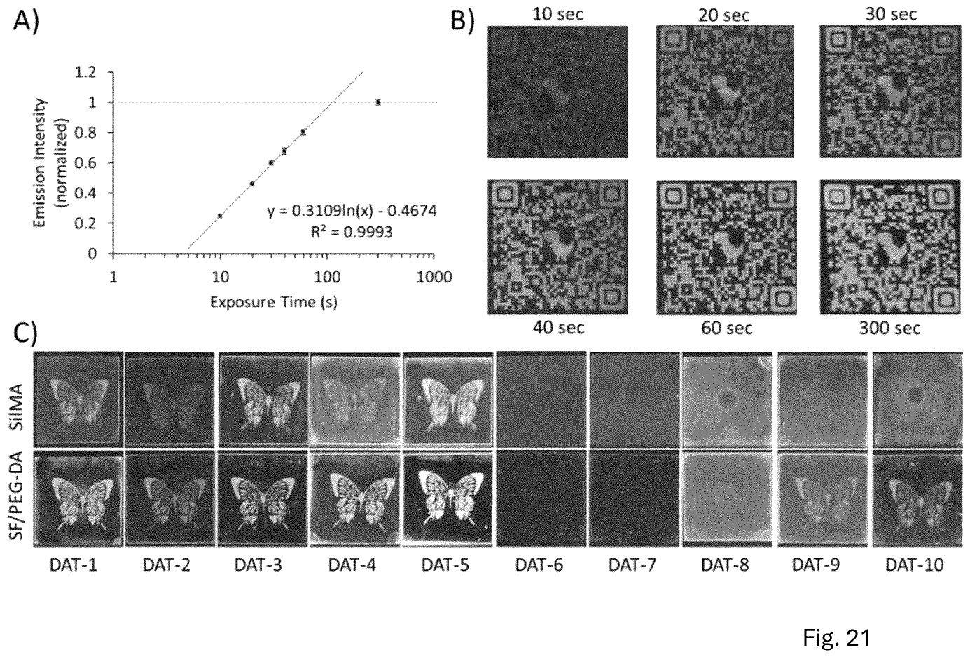

Films were fabricated with a spin-coating process, where either SilMA or a mixture of SF and PEGDA were dropped onto glass coverslips and spun to produce uniform films. Afterwards the films were water vapor annealed to recrystallize them and lock in the position of the polymer(s). Next a solution of the DAT dye in IPA or DI water was drop cast onto the surface, then spun to remove excess solution. This film was then exposed to 302 nm UV light through a quartz photomask or steel shadow mask for 30 seconds. The DAT impregnated films reacted promptly and attained 60% of their full fluorescent brightness in 30 seconds and their maximum brightness in less than 5 minutes (FIG. 21A,B). The different dyes produced colors analogous to their solution states. We also see that pyrazolines resulting from DAT 9 and DAT 10 are visible in PEG-DA/SF films and nearly completely quenched in SilMA films. This is likely due either the higher abundance of acrylate groups in SF/PEG-DA films, or better penetration into the films enabled by the PEG-DA.

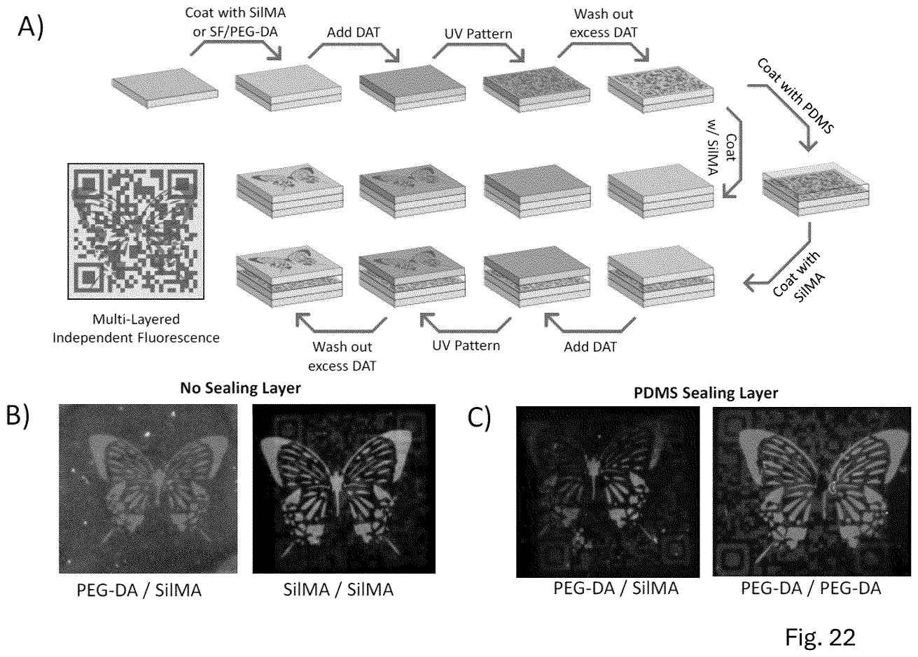

To create a film with multiple emission colors arising from the use of multiple DAT precursors, multilayer films were fabricated. (FIG. 22A) The first layer was created as previously described, then a second layer of SilMA is added, impregnated with DAT, patterned and washed. While this process works well with SilMA layers on both top and bottom, if PEG-DA is used in the lower layer, the patterned fluorescence will be almost entirely quenched (FIG. 22B). To prevent this, after the SF/PEG-DA layer is patterned and washed, an isolation layer of PDMS (Sylgard 184) is applied. With the isolation layer in place, subsequent layers of SilMA or SF/PEG-DA can be applied without quenching the lower layer (FIG. 22C).

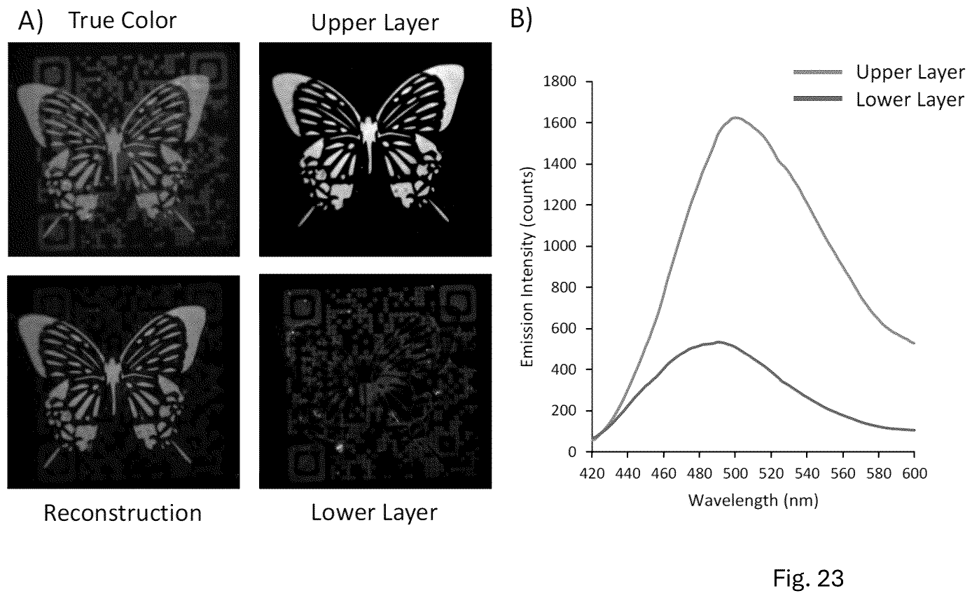

The multi-emitter films were imaged with a multi-spectral camera under 365 nm illumination to see if sufficient spectral separation was achieved to isolate the two images. The true color image could readily be separated into spectral components distinguished by both emission frequency and intensity (FIG. 23). The reconstructed image shows well separated signals, however the isolated image of the lower layer has a butterfly-shaped ghost image. There are two potential causes for this, either inter-layer crosstalk during patterning, or the upper layer is simply overshadowing the lower. This question can be resolved by altering the emission intensity of the upper layer by pH quenching.

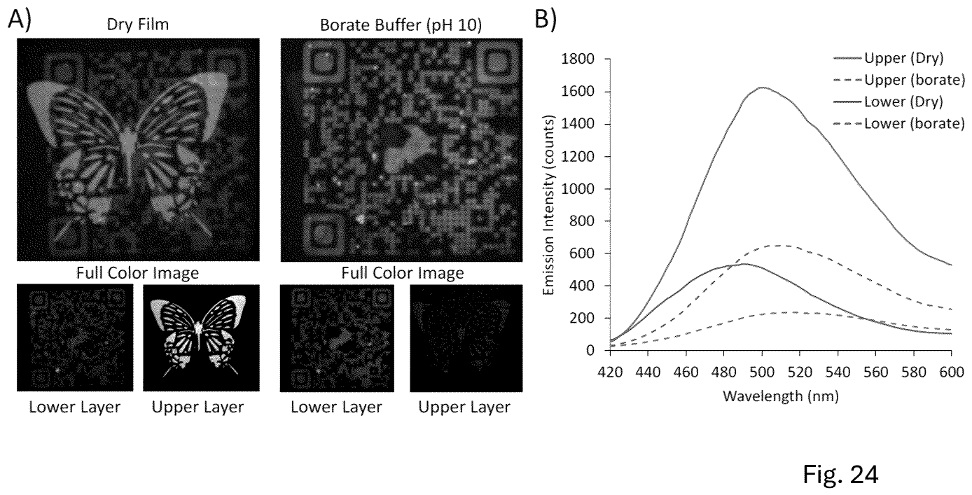

The pyrazolines remained pH sensitive in the solid films and their spectra could be shifted as by reaching pHs above or below pKa of the pyrazoline present in that film layer. To demonstrate this a film patterned with a Pyr-3 lower layer and Pyr-5 upper layer was imaged both dry and immersed in basic borate buffer (FIG. 24A). This caused a shift in the emission of both layers, a quenching of the upper layer, and a bathochromic shift in the lower layer (FIG. 24B). We can also see with the quenching of the upper layer that there is no evidence of cross-talk between the layers, and the lower layer is fully resolved in borate buffer.

### Tattoo Ink Fabrication

Successful photo-mediated patterning of the dyes ex vivo showed there was potential for intravital patterning of tattoo inks, however these faced additional challenges. First and foremost, the inks were no longer going to be part of a cohesive solid matrix, and thus would rely upon the matrix of the dermis to keep the spatial patterning intact. The dermis is a hydrated tissue and small molecular dyes would naturally diffuse through the interstitial fluid and would be further depleted by absorption and degradation by macrophages. To overcome this problem, I could use the same strategy that aesthetic tattoo inks use, using dye microparticles instead of small molecules. While the dyes themselves are not naturally suited to forming microparticles, SF is well suited to the task.

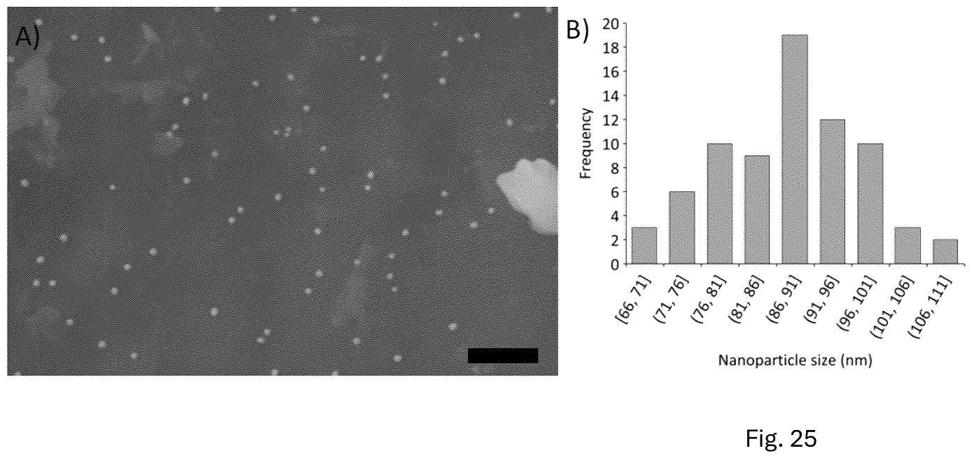

Two primary means of fabricating microparticles were pursued: solvent precipitation, and co-flow fabrication. Solvent precipitation was attractive due to the scalability of the technique, which involved mixing the SF solution into a solvent that would cause the recrystallization of the SF in solution. The solvent of choice was acetone, as this provided an intermediate speed of recrystallization (compared to the rapid recrystallization in methanol) that allowed the silk form nanoparticles instead of large-scale aggregates. These nanoparticles were isolated from solution by centrifugal pelleting and were repeatedly washed with DI water and re-pelleted to completely remove any acetone from the nanoparticles. This process was successful in producing nanoparticles (NPs), however they were typically sized in the order of ˜100 nm as measured by SEM (FIG. 25). This size is sufficient to prevent passive diffusion, however it is insufficient to resist macrophage clearance, which requires particle diameters of 800 nm or greater.

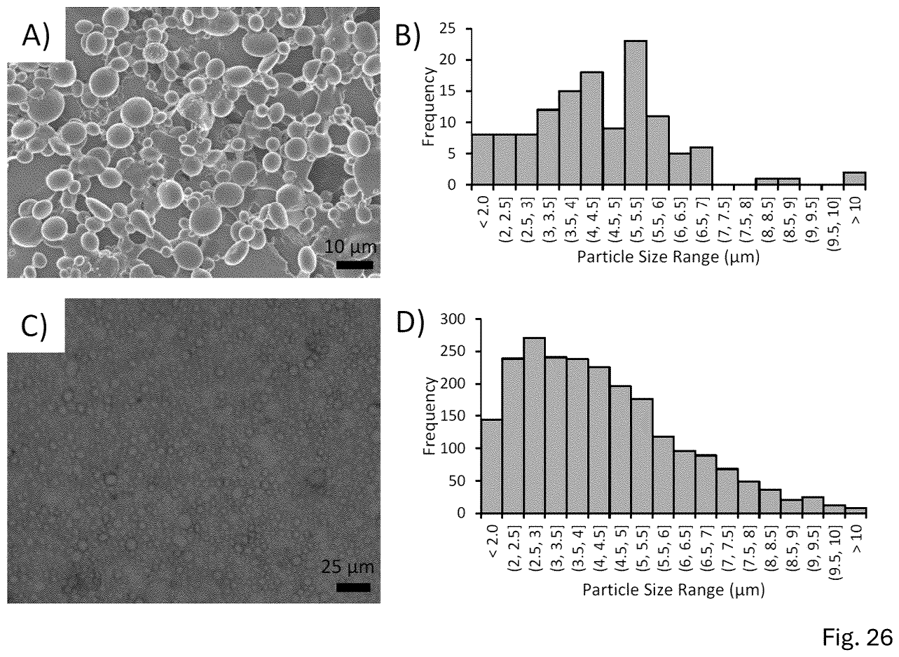

Co-flow fabrication was as performed using the SF/poly(vinyl alcohol) (PVA) solvent system established by Mitropoulos et al. (A. N. Mitropoulos, G. Perotto, S. Kim, B. Marelli, D. L. Kaplan, and F. G. Omenetto, “Synthesis of Silk Fibroin Micro- and Submicron Spheres Using a Co-Flow Capillary Device,” Advanced Materials, vol. 26, no. 7, pp. 1105-1110, 2014, doi: 10.1002/adma.201304244, which is incorporated herein in its entirety by reference for all purposes). A coaxial needle system was used with a 16-gauge outer needle containing the PVA continuous phase and a 27-gauge inner needle supplying the SilMA discrete phase. The continuous phase was flowed at 4 mL/hr while the discrete phase was flowed at 0.4 mL/hr, collected in a beaker, and subsequently dried on a PDMS sheet. The resulting film of PVA contained SF microparticles, which were isolated by redissolving the PVA in DI water, pelleted by centrifugation, and washed with DI water. The pelleting and washing were repeated 3 times to fully remove any residual PVA. This process resulted in the formation of much larger particles that were measured by optical and electron microscopy (FIG. 26). Image analysis of both optical and electron micrographs gave an average particle size of 4.0-4.4 μm in diameter. Results of the SilMA particle size analysis are located in Table 5.

TABLE 5

| | Optical Microscopy | Electron Microscopy |
|---|---|---|
| Mean Size (μm) | 4.43 | 4.38 |
| Median Size (μm) | 4.33 | 4.01 |
| Standard Deviation (μm) | 1.63 | 1.97 |

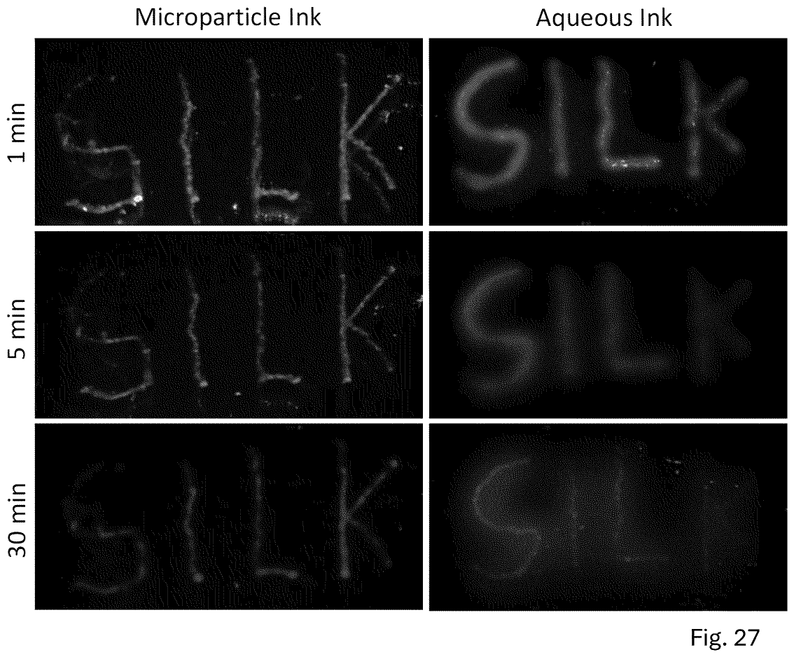

Once silk methacrylate microparticles (SilMA-MPs) were fabricated, they were loaded with DAT dye by pelleting the particles and resuspending them in DAT-laden solvent. The SilMA-MPs were allowed to absorb the dye, then residual dye was removed by centrifugal pelleting and water-washing. Due to the DAT's poor water solubility, sufficient DAT was able to be retained in the SilMA-MPs during washing to produce an intense visible fluorescence upon activation with 302 nm light. If the photoclick reaction were performed in vitro the DAT-laden SilMA-MPs (DAT-MPs) could be used as a traditional tattoo ink. To demonstrate this, DAT-MPs were exposed to 302 nm light for 5 minutes under constant stirring to achieve uniform activation. As a reference, aqueous SilMA was also mixed with DAT and activated by 5 minutes of 302 nm UV exposure under constant stirring. These dyes were both tattooed into 2% agarose gel imaging phantoms and visualized under 365 nm illumination (FIG. 27). Both achieved a visible pattern of fluorescence, however the aqueous SilMA very quickly diffused into the phantom, with notable blurring evident within minutes of tattooing and worsening to near-total loss of pattern fidelity in 30 minutes, evident at both the micro and macro scale. Conversely, the activated DAT-MP tattoos showed no fidelity loss over the same period, demonstrating complete resistance to dye diffusion.

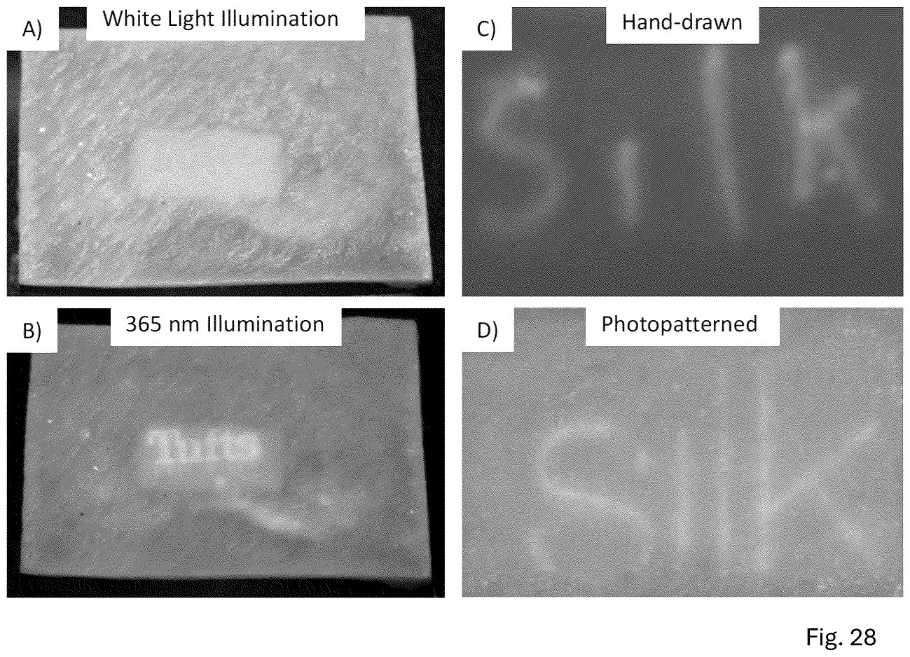

While use of the dye as a traditional tattoo ink is possible, it fails to utilize the photopatterning potential of the dye. It would be an attractive capability to pattern these dyes in situ, which gives the ability to apply very fine and detailed patterns without requiring the fine manual dexterity and years of training to become a skilled tattoo artist. Unfortunately, this requires patterning through the epidermis, which contains the UV-absorbing melanins that most humans possess. To prove this is possible, non-activated DAT-MPs were tattooed into porcine skin and the photoclick reaction was performed after tattooing (FIG. 28A,B). This patternability allowed me to use shadow masking to achieve finer patterns than I was able to manually tattoo (FIG. 28C,D). This highlights a key advantage of the ink in that it allowed me, an unskilled novice, to achieve tattoo patterns and fidelity that would otherwise require a highly skilled artisan.

Incorporation into Microneedle Arrays

The ability to pattern DAT-MPs post-tattooing allows for easy patterning, but also necessitates excessive tattooing to place ink in regions that is ultimately going to be unexposed. The chief problems with this are unneeded pain experienced by the recipient of the tattoo and unneeded time applying the tattoo by the artisan. It would be ideal if the bulk of the tattoo could be applied simultaneously and painlessly, then patterned later. This can plausibly be achieved with an SF-based microneedle array loaded with DAT-MPs. The dry yet non-crystalline SF provides the matrix for the needles, which is strong enough to penetrate skin but remains water soluble. Upon application the matrix is dissolved by the aqueous interstitial fluid within the dermis allowing any cargo contained within to be released below the dry epidermis.

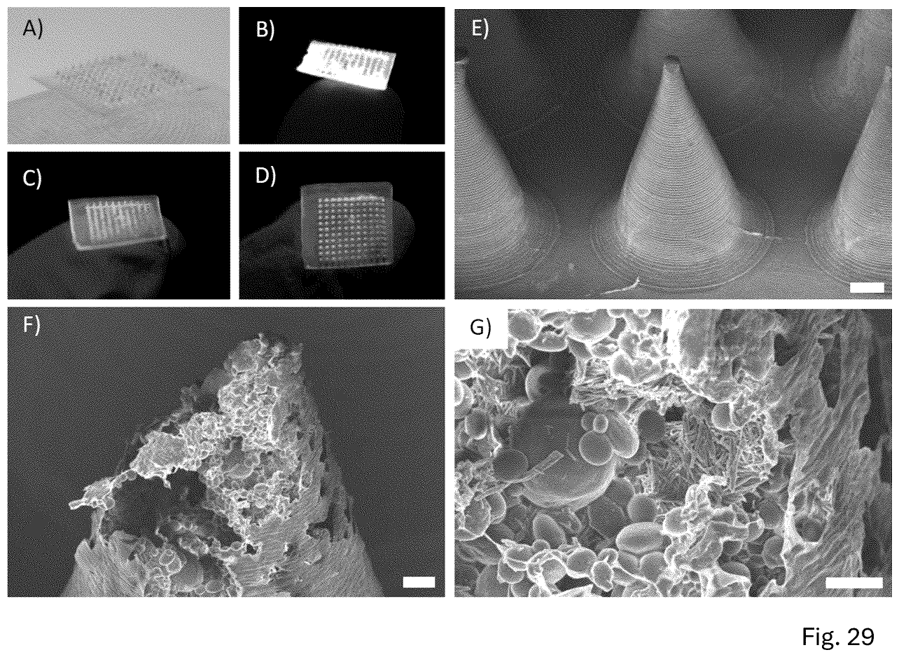

To fabricate this, an inverse mold of a microneedle array was fabricated out of PDMS. The chosen microneedle array contains conical needles 400 μm wide and 800 μm long, which are long enough to pierce the dermis but below the pain threshold of 1 mm that is required to activate dermal nerve endings in places like the outer arm. Into this mold was placed a suspension of DAT-MPs in unmodified SF. This suspension was loaded into the mold via centrifugation then allowed to dry in ambient conditions. After it was dried a second layer of pure SF was cast onto the mold to create a film to support the needles. This second layer was dried, and the microneedle array was manually demolded which yielded dry structures that retained the UV sensitivity even in the dry state (FIG. 29). These DAT-MP loaded arrays were imaged under SEM to observe surface morphology, and a broken needle revealed the internal structure of the array. The DAT-MPs incorporated into the array are visible within the broken needle, indicating they remain discrete from the SF of the matrix.

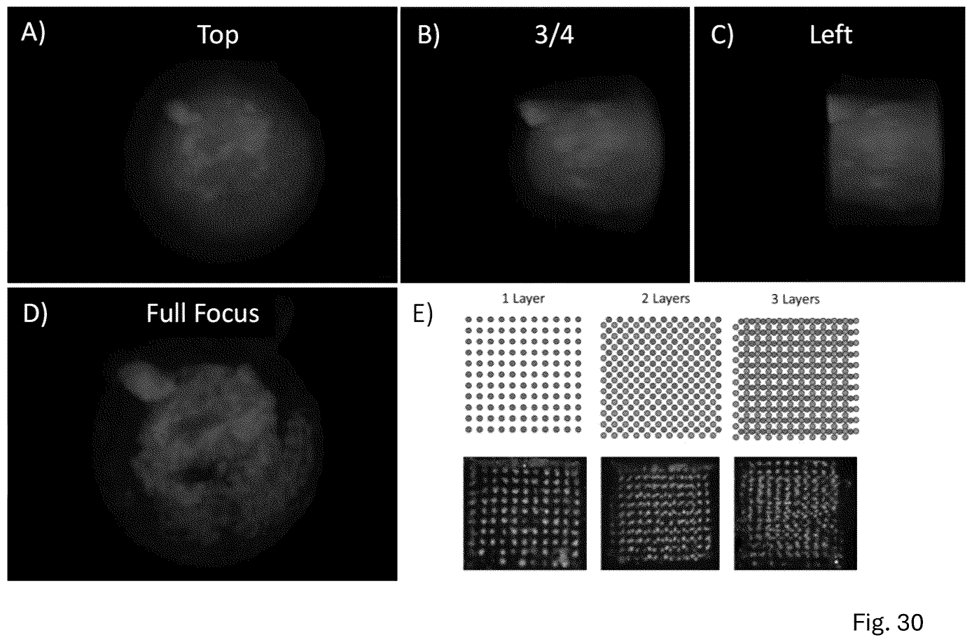

The delivery of DAT-MPs by the microneedle array was tested in an 2% agarose gel imaging phantom. The microneedle array was pressed into the surface with a glass slide behind it to provide even pressure, then held in place with a 500 g weight for 5 minutes. The slide was removed and the agarose gel was gently washed with DI water to remove any excess array material. A single needle was imaged under z-resolved fluorescent microscopy and a 3D reconstruction was made from the image stack (FIG. 30A-C). Individual fluorescent SilMA microparticles are visible in the needle, and when viewed from a three-quarters perspective or left profile, the conical shape pf the needle can be perceived. When the image stack was superimposed to make a full focus image, the nonspecific-blur seen in the 3D reconstruction is shown to be out of focus microparticles, indicating no freely diffusing fluorescent dye is present (FIG. 30D).

While MPs are needed to maintain tattoo pattern fidelity, they also resist diffusing after delivery by microneedle array (FIG. 30D). This leads to a clearly visible spotted pattern and also limits the amount of fine detail that that can be applied with subsequent photopatterning. To partially ameliorate this problem, microneedle arrays can be superimposed with a slight offset to embed needles in the spaces left by the previous impression. This effectively increases the resolution of the pattern and gives a more uniform appearance to the tattoo as a whole.

### Cytotoxicity

While the derivatives described in the literature have displayed exceptional biocompatibility, there is the possibility that the derivatives I have synthesized will introduce toxicity not seen in other analogs. The cytotoxicity and metabolic toxicity was tested by a combination of cell morphology and MTT assays in human dermal fibroblasts (hDFs). Primary hDFs are highly metabolically active stem cells and thus metabolism is a good gauge for population health. A common way to measure this is the MTT assay, in which cells are exposed to a potentially toxic compound and the media is supplemented with (3-(4,5-dimethylthiazol-2-yl)-2,5-diphenyltetrazolium bromide) (MTT), which is a substate that can be acted upon by mitochondrial oxidoreductase. This enzyme converts the yellowish MTT into a purple formazan product, whose concentration can be measured by UV-Vis absorbance. Healthy cells with active mitochondria will convert MTT to the formazan form rapidly, while metabolically inhibited cells will struggle to perform the conversion. This can detect cells under significant metabolic stress that have not yet died and thus would be overlooked by a simple live/dead assay.

Cellular morphology is an independent method of assessing cellular health. When healthy, cells like hDFs will adhere to flat plates in 2D culture and multiply rapidly, until they form a confluent layer. The pressure from surrounding cells tends to cause the cells to adopt an elongated and tubular morphology, often aligned with their neighboring cells. When cells are damaged, they will often suffer cytoskeletal damage and will lose their elongated morphology. Dead HDFs will be at the extreme end of this trend, appearing as very small spheres, devoid of cytoskeleton entirely.

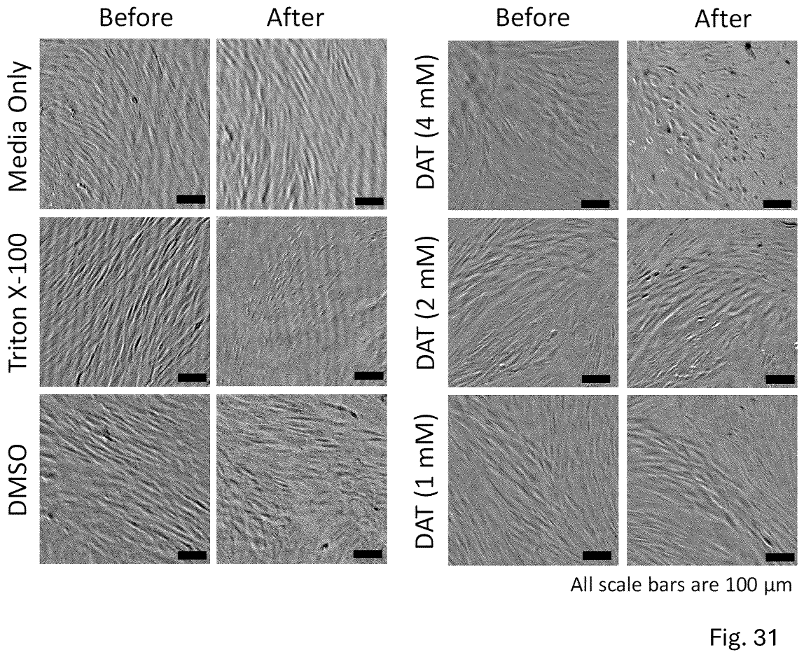

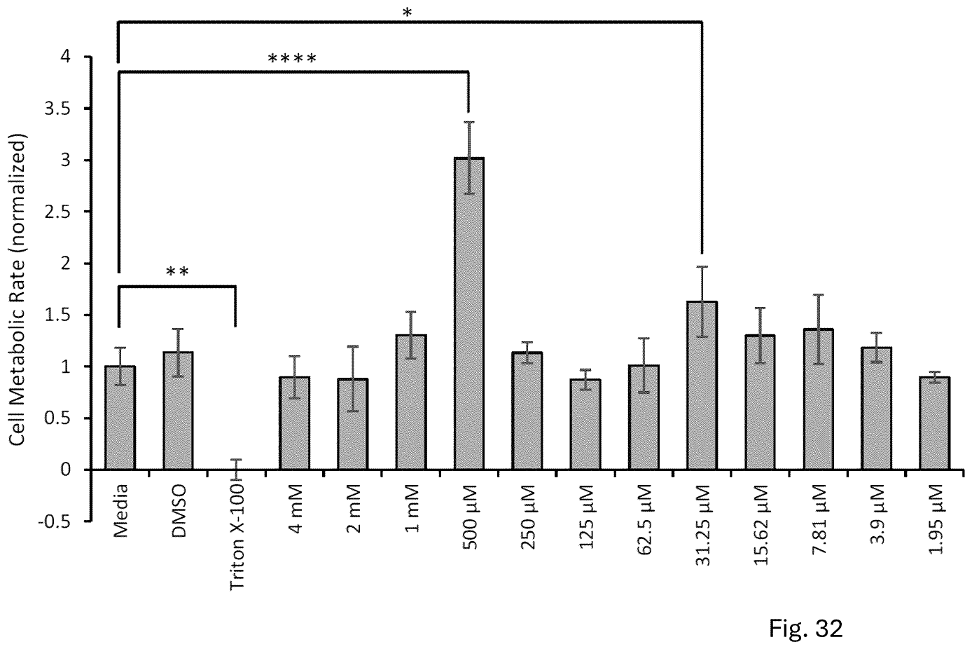

Initially DAT 1 was screened on a 2D assay, where hDFs were cultured in 96 well plates until confluent over the bottom of the well. These cells were then exposed to DAT 1 for 24 hours at a variety of concentrations from 4 mM—which is near saturation for growth media at 37 C—to 1 μM descending in a log 2 dilution series. Cellular morphology was examined by bright field transmission microscopy (FIG. 31), then metabolic activity was measured with a 4-hour MTT assay (FIG. 32).

The morphology of the hDFs prior to 24 bour incubation with DAT 1 was uniform across test conditions, displaying a continuous confluent layer of elongated cells (FIG. 32). After incubation with a known cytotoxic agent, the detergent Triton X-100, cells were small hard spheres indicative of dead cells. Media and vehicle maintained a confluent and elongated morphology that is expected from live hDF cells. The highest concentration of DAT 1 at 4 mM did appear to have some cytotoxic effects, with a mixed population of cells that appear as both shrunken and spherical as well as those that remain elongated. When the DAT 1 was reduced to a concentration of 2 mM, cellular morphology was largely elongated and well attached indicating the majority of the cells were living and active. All concentrations of DAT lower than 2 mM were visually indistinguishable from the media-only controls, suggesting minimal cytotoxicity at these concentrations.

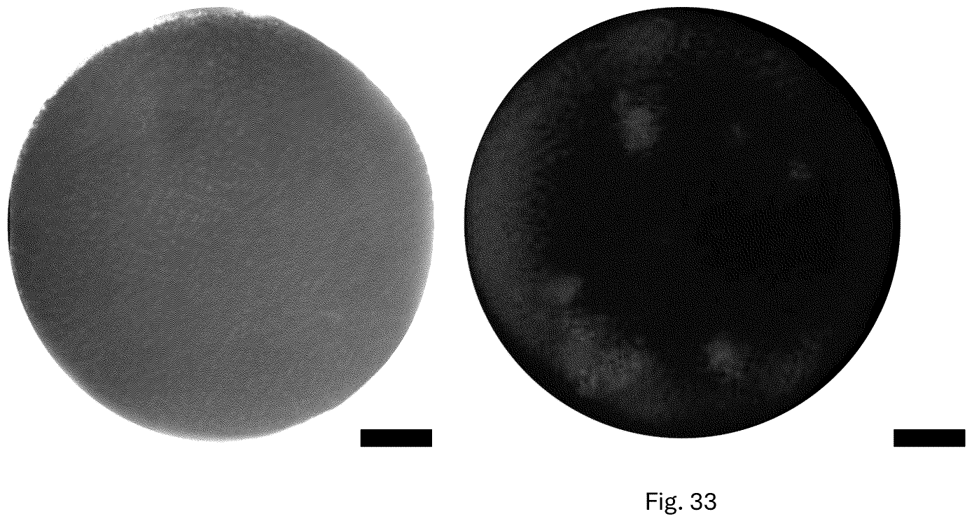

The metabolic data from the MTT assay largely agrees with morphological data with some caveats. The vehicle (1% v/v DMSO) was not seen to have any toxicity while Triton X-100 completely destroyed the metabolic capacity of the cells. There was a slight, but statistically insignificant loss in metabolic capacity from the highest DAT-1 loading, however lower concentration loadings were unusual. There was greater conversion of the MTT to formazan in cells treated with DAT 1 in moderate concentration. This overall trend was seen across multiple plates, with cells in the central wells generally outperforming cells on the exterior of the plate. This is likely due to plate-edge effects from evaporation or uneven gas exchange. Steps were taken to prevent this, such as not utilizing any wells adjacent to the exterior of the plate and instead filling these wells with sterile DPBS, but the trend remained. This artificial bias placed an additional stress on cells further from the center, which includes the negative and vehicle controls. This skews the result in to look less toxic than it might otherwise have been, thus the metabolic toxicity data from 4 mM and 2 mM DAT 1 is likely higher than indicated in the MTT assay. To more accurately simulate conditions in skin, cells were also cultured in a 3D scaffold environment that simulates a skin-like environment (FIG. 33).

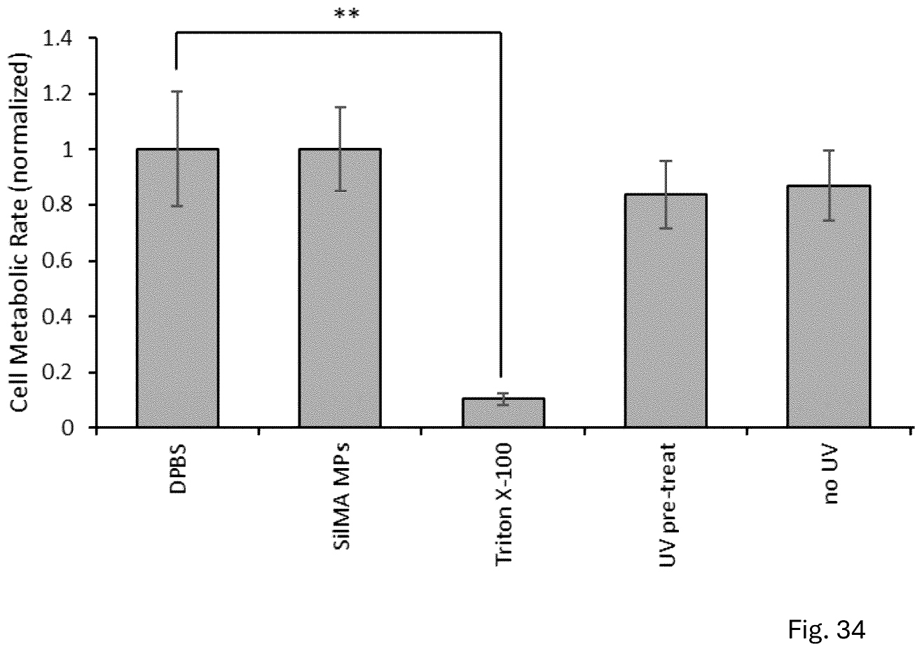

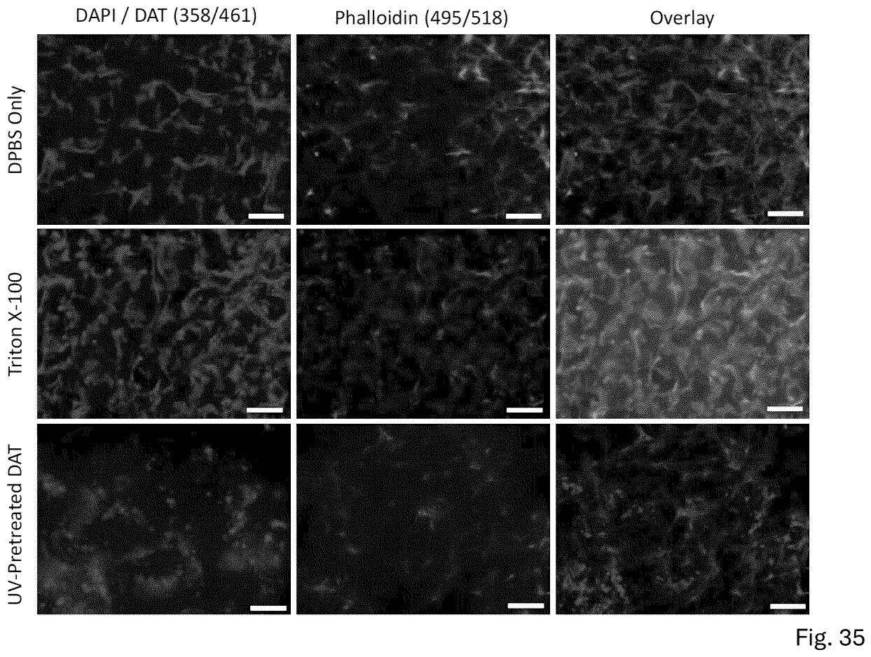

The 3D silk scaffolds here are based on dermal model scaffolds developed and characterized by Adelfio et al. (M. Adelfio et al., “Three-Dimensional Humanized Model of the Periodontal Gingival Pocket to Study Oral Microbiome,” Advanced Science, vol. 10, no. 12, p. 2205473, 2023, doi: 10.1002/advs.202205473, which is incorporated herein by reference for all purposes). These scaffolds recapitulate a skin like environment by having a closed-cell structure on one side simulating the epidermis and an open-cell architecture on the other, allowing nutrient flux and simulating the dermis. Scaffolds were fabricated by lyophilizing 5% w/v SF solution, then autoclaving the scaffold to fully recrystallize the scaffold. The scaffold was then cut to size, sterilized and coated with collagen to promote cell adhesion and seeded with human dermal fibroblasts (hDFs). Scaffolds were then tattooed by injecting 1 μL of solution into the gel at 6 points around near the outer edge and one in the centure, forming a symmetrical radial pattern. The scaffolds were incubated with the tattoos for 7 days then divided into two populations. The first were subjected to the MTT assay (FIG. 34), the second were fixed and stained (FIG. 35). Color versions of these images can be located in Ostrovsky-Snider and will be provided to an examiner upon request.

Stained scaffolds were imaged with z-resolved fluorescence microscopy, with both DAPI and the active DAT-MPs fluorescing brightly on the 358/461 channel. The DAPI also bound strongly to the SF scaffold causing very strong background fluorescence and making nuclei very hard to discern. Thus, DAPI serves largely to show the location of the silk scaffold. We see in the DPBS tattooed scaffold that cells are residing in the scaffold pores and have the expected elongated morphology of healthy fibroblasts. In the same treated with the cytotoxic Triton X-100, we see only background fluorescence due to non-specific adsorption of both phalloidin and DAPI on the scaffold, with both images superimposing on one another. In scaffolds tattooed with the DAT-MPs, the fluorescence of the microparticles overwhelms the DAPI in both nuclei and on the scaffold, however the phalloidin reveals fibroblasts showing a healthy morphology in close contact with the microparticles. This indicates that there is likewise no detectable localized cytotoxicity from the microparticles.

### Conclusion

Here I have demonstrated that the DAT-photoclick reaction can be combined and used with SilMA or SF/PEG-DA composites to create patterned fluorescent films and coatings with multispectral fluorescence emission and independent control of coating patterns. These patterns still display pH sensitivity of their emission and can be used to deconvolute intermixed patterns when used in conjunction with multispectral imaging.

Furthermore, the SilMA can be made into microparticles and infused with DAT photoreactive dye precursor to make a tattoo ink that can be applied like a traditional tattoo, or applied in a non-patterned manner and patterned after application with the photoreactive process. These inks are insensitive to diffusion and can be patterned in skin despite the UV-absorbing melanin contained in the epidermis. These inks may also be loaded into microneedle arrays and applied over large areas simultaneously and painlessly. These arrays may be patterned post-application as well, although the diffusion resistance of the dye requires multiple microneedle applications to achieve continuous coverage of the area.

Lastly the dye and the photoactive DAT precursor show low cytotoxicity, except in very high concentrations. Cells show minimal metabolic toxicity and normal morphology in all but near-saturation levels of precursor exposure. The SilMA-MP vehicle and ex vivo-reacted dye show no measurable cytotoxicity, and tattoos remained visible and fluorescent after 7 days in culture showing they are incentive to enzymes and reactive biological chemicals that may quench their fluorescence.

### Future Directions

There is ample opportunity for future exploration in this area, as many questions remain to be answered. Firstly, while tissue models showed minimal toxicity, whole organisms behave in a vastly different way due to the presence of multiple cell types, the immune system and enzymatic conversion of implanted chemicals via hepatic enzymes. These factors may alter the toxicity of the tattoo constituents on a tissue, organ or organismal level. The compounds may also pose risks for carcinogenicity, depending on how they distribute in the cellular environment. If they are readily up taken by cells, and enter the cytoplasm, then they may be able to enter the nucleus and potentially act as intercalating agents, thus causing replication errors in dividing cells. The literature does not suggest this is the case, but it remains a possible that must be actively excluded if these dyes and precursors are to be considered truly biocompatible. The best way to do this is with long-term live animal testing to see the effects on the whole organism.

### Broader Impacts

Altogether, the works presented here expand the knowledge of the interactions of silk with the reactants and products of tetrazole-ene photoclick reactions, and layout a method for designing fluorescent constructs of desired emissive properties, including emission wavelength and pH-based emission shifting or quenching. These reactants can then be grafted to the SF backbone to make potential photocrosslinkable materials via the Gomberg-Bachmann reaction. Finally, these materials can be used to create biocompatible photoreactive ink precursors that can be formed into fluorescent markings before or after application to biological systems. This has the potential to make selectively visible tattoos for biomedical and aesthetic purposes, as well as anti-counterfeiting marks on bio-interfacing materials like drugs and implants.

The tetrazole/alkene photoclick reaction is already thoroughly documented and extensively used in many biochemical applications, however the research here broadens and extends that work with regards to the fluorescent products and the effects substitution has on the reactivity of the tetrazole derivatives.

Works by An et al. have already documented the direct correlation between the electronegativity of the substituents in the para position of the 2-phenyl group of the DAT and the fluorescence of the resulting pyrazolines. This work extended the same treatment to the para and meta substituted positions on the 5-phenyl group of the DAT. This revealed that electronegativity had the opposite trend, and infact highly electronegative groups produced a bathochromic shift at the para position. It also demonstrated that nearly all of these effects were driven by resonant electro delocalization, with highly electron withdrawing and donating groups at the meta position producing negligible impacts on the fluorescence of the resulting pyrazolines. These effects could be modulated by altering the ionization state of the substituents via environmental pH, thus allowing for fluorescent sensing of pH. This further strengthens the evidence for the mechanism proposed here and allows for additional levers of controls in the rational design of fluorophores.

Scientifically, this will allow for the creation of molecular probes with precisely tuned fluorescent properties, allowing for the creation of a multitude of dyes across the visible spectrum. If paired with bulky adducts at the meta positions in the phenyl rings similar to those developed by An et al. that limit solvent access to the molecular core, this could allow access to fluorophores that emit in the red and near-IR bands, further broadening the scope of fluorescent molecules. Alternatively, the quenching by polar solvents could be embraced as a mechanism for switchable fluorescence in molecular probes. Ionizable groups on both the 2 and 5 phenyl position could be used in concert to tune the pH of quenching, thus producing a pH-tunable on-off fluorescent probe.

The utility of the photoclick reaction has the potential to extend beyond creating fluorescent probes and into creating photorheological or photocrosslinkable silk fibroin materials, if the DATs can be covalently bound to the fibroin. In this work, it was demonstrated that Gomberg-Bachmann reactions were better suited for this reaction compared to carbodiimide and azo-dye reactions. This unique reaction takes advantage of the reactivity of the azo-group for the highly abundant aryl groups of tyrosine rather than the amines or carboxylates targeted by carbodiimide reactions, while avoiding the creation of a chromophore from the azo-dye reaction.

The creation of a photocrosslinkable bulk SF polymer could provide many scientific and commercial applications. Firstly, as the crosslinking mechanism is a photoclick reaction, it avoids a limiting factor of most photocrosslinkable polymers in use today, the free-radical. While free-radical polymerization is very robust, it is sensitive to oxygen and radical scavengers that prematurely terminate the reaction. This becomes a limiting factor when printing exposed to the atmosphere, as the diffusion of atmospheric oxygen rapidly quenches the crosslinking reaction in very thin or small components. The photoclick reaction is unaffected by dissolved oxygen and would be uninhibited in these circumstances, allowing reaction in highly oxygenated conditions. This could prove highly useful for 3D printing of very thin components when exposed to an atmospheric interface. It may also aid in 3D bioprinting, as the free-radicals and photoinitiators that create them cause oxidative stress to the cells suspended in the printing medium.

A useful biproduct of this research is the creation and validation of a GPC method for characterization of SF. This method uses standard calibration, allowing it to be performed on any HPLC instrument, not merely those equipped with specialized light-scattering detectors. The standards for this technique are all agricultural biproducts and are therefore cheap and easily sourced, removing yet another cost barrier. The added resolution this technique provided also allowed us to demonstrate that boiling time during degumming provides precise control over molecular weight of SF. As many properties of bulk SF solids are closely tied to the molecular weight of the regenerated polymer, this research provided a simply universal calibration curve for achieving particular molecular weight ranges by altering the boil time during degumming.

The incorporation of the DAT/SilMA system into a tattoo-able ink demonstrated the ability to make a biocompatible ink that can be delivered by microneedle array and patterned post-application. The DAT chemical moiety is generally considered to have low toxicity, but the exact threshold of toxicity for this derivative of the compound had yet to be determined before this work. The method for fabricating the ink is highly flexible and is readily adaptable to all other tetrazole derivatives, making the use of other DATs for this process a simple ingredient substitution. The interplay of the fluorescent behavior of pyrazolines with silk fibroin had also never been performed before. Here we demonstrated that PEG-DA in isopropyl alcohol reflects the behavior of SilMA based liquid and solid systems, in both emissive properties and pH response. This allows for substitution of the alkene-containing polymer without dramatic effect on the fluorescent behavior.

This has several applications as a potential biomedical product, as well as a commercial product for aesthetic use. In the biomedical context, it could be used to create tattoos for laboratory animals that can be patterned at the time of application, thus simplifying the production and flexibility of the labels. They could also potentially be used as radiotherapy fiducials for humans undergoing cancer treatments. The marking are superior to traditional tattoo inks as the markings will only be visible under ultraviolet illumination and thus can be placed in visible locations on the patient without causing emotional stress for the patient from the potential social stigmas of visible tattoos. These inks could also be used for niche aesthetic tattooing, creating selectively visible tattoos. Their biocompatible organic composition makes them superior to other fluorescent dyes that relied on toxic inorganic materials to perform the same task. Their proteinaceous composition also means the microparticles are likely subject to biodegradation and resorption within the span of a year. This itself is an unexploited niche within the tattoo world, as there exists a substantial gulf between permanent tattoos, which remain visible for life, and temporary tattoos, which fade completely within 2-3 weeks. This may also make visible absorptive dyes blended with SilMA MPs attractive as intermediate-length, fully visible tattoos.

The biocompatible nature of the microparticles, as well as their ability to pattern them makes them ideal for anti-counterfeiting applications in degradable or bio-interfacing applications. The films make be loaded with dyes and readily patterned to create selectively visible labels of arbitrary pattern. These patterns may also display pH sensitivity and thus offer a challenge-response pair for added security. This idea may also be expanded to DAT-loaded SilMA-MPs suspended in a SF film, to create fluorescent patterns with a unique and random microstructure, thus creating a physically unclonable function (PUF). These multiple independent layers of security, in addition to the low cost for fabricating these fluorescent dyes when compared to other methods such as utilizing fluorescent proteins, makes them ideal candidates for anti-counterfeiting marks for drugs and consumable/implantable items.

These innovations together have the potential for lowering the cost and increasing the application-space of fluorescent markings, like those already in use for anti-counterfeiting of currency. The ease and availability of post-application patterning could create an expansion of the uses of fluorescent markings as all materials documented here are readily producible at scale bulk production could potentially make these inks price-competitive with commercial offerings. Beyond the application laid out here, there remains untapped potential for using these marks in unexpected and unconventional ways if brought to market with a sufficiently low price tag, which can be achieved with production at scale.

1. A silk photoclick tattoo ink comprising:
modified silk nanoparticles comprising modified silk fibroin functionalized with a plurality of first photoclick chemistry pair moieties, wherein the modified silk nanoparticles are biocompatible and bioresorbable; and
photoclick chromophores comprising a second photoclick chemistry pair moiety, wherein the first photoclick chemistry pair moiety and the second photoclick chemistry pair moiety undergo a known photoreaction when illuminated with light having a predetermined wavelength for a predetermined exposure length and a predetermined exposure intensity, thereby covalently bonding at least a portion of the photoclick chromophores to at least a portion of the modified silk nanoparticles,
wherein the silk photoclick tattoo ink is safe for human use as a tattoo ink.

2. (canceled)

3. (canceled)

4. A method of generating a silk photoclick tattoo in a subject's skin, the method comprising the following steps:
a) administering a silk photoclick tattoo ink to an area of the subject's skin, the silk photoclick tattoo ink comprising modified silk nanoparticles and photoclick chromophores, the modified silk nanoparticles comprising modified silk fibroin functionalized with a plurality of first photoclick chemistry pair moieties, wherein the modified silk nanoparticles are biocompatible and bioresorbable, the photoclick chromophores comprising a second photoclick chemistry pair moiety;
b) exposing the silk photoclick tattoo ink within the area to light having a predetermined wavelength for a predetermined exposure length and a predetermined exposure intensity, thereby initiating a known photoreaction and covalently bonding at least a portion of the photoclick chromophores to at least a portion of the modified silk nanoparticles,
the administering and exposing of steps a) and b) thereby generating the silk photoclick tattoo in the area of the subject's skin.

5. The method of claim 4, the method further comprising the following steps:
c) administering the silk photoclick tattoo ink to a delimited area of the subject's skin;
d) preventing the silk photoclick tattoo ink within the delimited area from undergoing the known photoreaction; and
e) optionally waiting a bioresorption length of time,
the administering, preventing, and optionally waiting of steps c), d), and e) generating an absence of the silk photoclick tattoo in the delimited area.

6. The method of claim 5, wherein the preventing of step d) includes applying a photomask to the delimited area, wearing a protective sheath covering the delimited area, selectively illuminating, or a combination thereof.

7. The method of claim 5, the method comprising the following step: e) waiting the bioresorption length of time.

8. The method of claim 7, wherein the bioresorption length of time is between 1 month and 1 year.

9. The method of claim 4, the method further comprising administering a selective degradation agent to at least a portion of the area, thereby dissolving at least a portion of the silk photoclick tattoo ink within the at least a portion of the area, thereby reducing a visibility of the silk photoclick tattoo within the at least a portion of the area.

10. The method of claim 9, wherein the selective degradation agent is an enzymatic agent.

11. The method of the claim 10, wherein the enzymatic agent is protease XIV, trypsin, bromelain, papain, or a combination thereof.

12. The method of claim 4, wherein the administering of step a) comprises administering via a dissolvable microneedle.

13. The method of claim 4, wherein the method comprises administering two different inks to two different areas, wherein the two different inks have different optical properties.

14. The method of claim 4, wherein the method comprises selectively administering inks with different optical properties to different areas, thereby generating images with varying optical properties across the different areas.

15.-22. (canceled)

23. A method of generating a silk photoclick volumetric image within a volume of an article, the method comprising the following steps:
a) administering a silk photoclick article ink to a volume, the silk photoclick article ink comprising modified silk nanoparticles and photoclick chromophores, the modified silk nanoparticles comprising modified silk fibroin functionalized with a plurality of first photoclick chemistry pair moieties, wherein the modified silk nanoparticles are biocompatible and bioresorbable, the photoclick chromophores comprising a second photoclick chemistry pair moiety;
b) exposing the silk photoclick article ink within the volume to light having a predetermined wavelength for a predetermined exposure length and a predetermined exposure intensity, thereby initiating a known photoreaction and covalently bonding at least a portion of the photoclick chromophores to at least a portion of the modified silk nanoparticles,
the administering and exposing of steps a) and b) thereby generating the silk photoclick volumetric image within the volume of the article.

24.-30. (canceled)

31. The method of claim 4, wherein the modified silk nanoparticles have an average particle diameter of between 0.1 and 50 micrometers.

32. The method of claim 4, wherein the modified silk nanoparticles have an average volumetric density of the plurality of first photoclick chemistry pair moieties of 10-15%.

33. The method of claim 4, wherein the plurality of first photoclick chemistry pair moieties comprises, consists essentially of, or consists of methacrylate moieties and the second photoclick chemistry pair moiety is a diaryl-tetrazole moiety.

34. The method of claim 4, wherein the second photoclick chemistry pair moiety is a 2,5-diaryl-tetrazole moiety.

35. The method of claim 4, wherein the covalent bonding activates a fluorescence within the silk photoclick tattoo ink.

36. The method of claim 4, wherein the photoclick chromophores have the following formula:

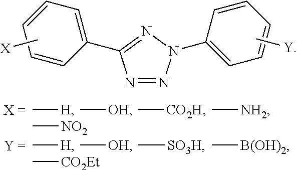

37. The silk method of claim 4, wherein the photoclick chromophores are selected from the following group: [chemical structure]
